SEQUENCE LISTING (67 sequences)

SEQ ID NO: 1 - DNA, 444 bp, Artificial Sequence
  HBV truncated core antigen gene
gacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtc

SEQ ID NO: 2 - protein, 148 aa, Artificial Sequence
  HBV truncated core antigen
DIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 3 - DNA, 447 bp, Artificial Sequence
  HBV truncated core antigen gene
atggacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatccaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggatccgcactccccctgcttatcgaccacccaacgcaccaattctgtccaccctgcccgagaccacagtggtc

SEQ ID NO: 4 - protein, 149 aa, Artificial Sequence
  HBV truncated core antigen
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 5 - DNA, 2529 bp, Artificial Sequence
  HBV pol antigen gene
atgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcacacgccacggggatgagtccttttgccagcagtctagtggcatcctgagcagatcccccgtggggccttgtctgcagtctcagctgcggaagagtagactgggactgcagccacagcagggacacctggcacgacggcagcagggaaggtctggcagtatccgggctagagtgcatcccacaactagaaggcctttcggcgtcgagccatcaggaagcggccacaccacaaacaccgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 6 - DNA, 2529 bp, Artificial Sequence
  HBV pol antigen gene
atgcccctgtcttaccagcactttagaaagctgctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcacacgccacggggatgagtccttttgccagcagtctagtggcatcctgagcagatcccccgtggggccttgtctgcagtctcagctgcggaagagtagactgggactgcagccacagcagggacacctggcacgacggcagcagggaaggtctggcagtatccgggctagagtgcatcccacaactagaaggcctttcggcgtcgagccatcaggaagcggccacaccacaaacaccgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 7 - protein, 843 aa, Artificial Sequence
  HBV pol antigen
MPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 8 - DNA, 63 bp, Artificial Sequence
  Cystatin S signal peptide coding sequence
atggctcgacctctgtgtaccctgctactcctgatggctaccctggctggagctctggccagc

SEQ ID NO: 9 - protein, 21 aa, Artificial Sequence
  Cystatin S signal peptide sequence
MARPLCTLLLLMATLAGALAS

SEQ ID NO: 10 - DNA, 378 bp, Artificial Sequence
  triple enhancer regulatory sequence
ggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagac

SEQ ID NO: 11 - DNA, 12 bp, Artificial Sequence
  linker coding sequence
gccggagctggc

SEQ ID NO: 12 - DNA, 248 bp, Artificial Sequence
  ApoAI gene fragment
ttggccgtgctcttcctgacgggtaggtgtcccctaacctagggagccaaccatcggggggccttctccctaaatccccgtggcccaccctcctgggcagaggcagcaggtttctcactggccccctctcccccacctccaagcttggcctttcggctcagatctcagcccacagctggcctgatctgggtctcccctcccaccctcagggagccaggctcggcatttcgtcgacaagcttagccacc

SEQ ID NO: 13 - DNA, 130 bp, Artificial Sequence
  SV40 polyadenylation signal sequence
aacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 14 - DNA, 81 bp, Artificial Sequence
  immunoglobulin secretion signal coding sequence
atggagttcggcctgtcttgggtctttctggtggcaatcctgaagggcgtgcagtgtgaagtgcagctgctggagtctgga

SEQ ID NO: 15 - protein, 27 aa, Artificial Sequence
  immunoglobulin secretion signal sequence
MEFGLSWVFLVAILKGVQCEVQLLESG

SEQ ID NO: 16 - protein, 996 aa, Artificial Sequence
  HBV core-pol fusion antigen sequence
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVAGAGMPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 17 - protein, 1023 aa, Artificial Sequence
  HBV core-pol fusion antigen sequence with Ig signal sequence
MEFGLSWVFLVAILKGVQCEVQLLESGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVAGAGMPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 18 - DNA, 584 bp, Artificial Sequence
  hCMV promoter
tgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagc

SEQ ID NO: 19 - DNA, 684 bp, Artificial Sequence
  hCMV promoter sequence
accgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgg

SEQ ID NO: 20 - DNA, 225 bp, Artificial Sequence
  bGH polyA signal
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 21 - DNA, 671 bp, Artificial Sequence
  pUC ORI
cccgtagaaaagatcaaaggatcttcttgagatcctttttttctgcgcgtaatctgctgcttgcaaacaaaaaaaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 22 - DNA, 795 bp, Artificial Sequence
  KanR coding sequence
atgattgagcaagatggtcttcacgctggctcgccagctgcgtgggtggaacgcctgtttggttatgattgggcgcagcagactattggatgttccgacgcggctgtatttcggctgtctgctcagggtcgccccgtgctgtttgtgaagacggatttgtctggcgcattaaatgagttacaggacgaggcggctcgtctgagttggttggccaccaccggcgtgccctgcgccgcagtgctggatgtcgtgacagaagcaggccgcgattggctccttctcggcgaagtgccgggccaggacctgctcagcagccacttggcaccggcagaaaaagtttctatcatggccgacgccatgcgtcgtcttcacactctcgatccggccacgtgcccctttgaccaccaggccaagcatcgtattgaacgtgcgcgtactcggatggaagcaggtttagtagaccaggacgatttggatgaggaacatcaaggcctggccccggctgaactgtttgcgcgcttaaaagcgtcgatgccagatggcgaagatttggtagtcacccatggagatgcgtgtttgccaaacatcatggttgaaaatggccgcttctcaggctttattgactgtgggcgcctgggtgttgccgaccgctatcaagatattgcgctcgcaactcgtgacatcgctgaagagctgggcggagaatgggctgaccgtttcctggtactgtatggcattgcagcgcccgattcccaacgcatcgcattttatcgtctgctggatgagtttttctaa

SEQ ID NO: 23 - protein, 264 aa, Artificial Sequence
  Codon optimized Kanr
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 24 - DNA, 99 bp, Artificial Sequence
  bla promoter
acccctatttgtttatttttctaaatacattcaaatatgtatccgctcatgagacaataaccctgataaatgcttcaataatattgaaaaaggaagagt

SEQ ID NO: 25 - DNA, 8364 bp, Artificial sequence
  alphavirus genome sequence
taatacgactcactatagagaggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcatcgatacagcagcaattggcaagctgcttacatagaaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaacccctctctaaacggaggggtttttttcagcgtaactggactggccacagttaggcggccgcgcatgttcatcatcagtaacccgtatcgtgagcatcctctctcgtttcatcggtatcattacctccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatg

SEQ ID NO: 26 - DNA, 50 bp, Artificial sequence
  5′ UTR of Aura virus
atagcggacggactagtacttgtactacagaattaactgccgtgtgccgc

SEQ ID NO: 27 - DNA, 50 bp, Artificial sequence
  5′ UTR of Chikungunya virus
atggctgcgtgagacacacgtagcctaccagtttcttactgctctactct

SEQ ID NO: 28 - DNA, 50 bp, Artificial sequence
  5′ UTR of O′nyong-nyong virus
atagctgcgtgatacacacacgcagcttacgggtttcatactgctctact

SEQ ID NO: 29 - DNA, 50 bp, Artificial sequence
  5′ UTR of Bebaru virus
atggcggctgtgtgacacacgagccgtcgatttcaaccttcttgctccct

SEQ ID NO: 30 - DNA, 50 bp, Artificial sequence
  5′ UTR of Semliki forest virus
atggcggatgtgtgacatacacgacgccaaaagattttgttccagctcct

SEQ ID NO: 31 - DNA, 50 bp, Artificial sequence
  5′ UTR Mayaro virus
atggcgggcaagtgacacttgttccgccggtcgtctctaagctcttcctc

SEQ ID NO: 32 - DNA, 50 bp, Artificial sequence
  5′ UTR of Getah virus
atggcggacgtgtgacatcaccgttcgctctttctaggatcctttgctac

SEQ ID NO: 33 - DNA, 50 bp, Artificial sequence; identical to SEQ ID NO: 32
  5′ UTR of Sagiyama virus

SEQ ID NO: 34 - DNA, 50 bp, Artificial sequence
  5′ UTR Ndumu virus
atggtgcggagttgagagacgaagcaccaaacaactacgcggctcaccat

SEQ ID NO: 35 - DNA, 50 bp, Artificial sequence
  5′ UTR of Middelburg virus
attggtggttacgtacacgtgccaccaccccccaccctccaagcgatcca

SEQ ID NO: 36 - DNA, 50 bp, Artificial sequence
  5′ UTR of Eastern equine encephalitis virus
atagggtacggtgtagaggcaaccaccctatttccacctatccaaaatgg

SEQ ID NO: 37 - DNA, 50 bp, Artificial sequence
  5′ UTR of Fort Morgan virus
atagggtatggtttagaggcgcctaccctacttaaccgatccaaacatgg

SEQ ID NO: 38 - DNA, 50 bp, Artificial sequence; identical to SEQ ID NO: 37
  5′ UTR of Buggy Creek virus

SEQ ID NO: 39 - DNA, 50 bp, Artificial sequence
  5′ UTR of Venezuelan equine encephalitis virus
atgggcggcgcaagagagaagcccaaaccaattacctacccaaaatggag

SEQ ID NO: 40 - DNA, 50 bp, Artificial sequence
  5′ UTR of Whataroa virus
attggcggcatagtacatactatataaaagaaacagccgaccaattgcac

SEQ ID NO: 41 - DNA, 50 bp, Artificial sequence
  5′ UTR of Sindbis virus
attgacggcgtagtacacactattgaatcaaacagccgaccaattgcact

SEQ ID NO: 42 - DNA, 50 bp, Artificial sequence
  5′ UTR of Bebaru virus
attggcggcgtagtacacactattgaatcaaacagccgaccaattgcact

SEQ ID NO: 43 - DNA, 145 bp, Artificial sequence
  DLP from Sindbis virus
atagtcagcatagtacatttcatctgactaatactacaacaccaccaccatgaatagaggattctttaacatgctcggccgccgccccttcccggcccccactgccatgtggaggccgcggagaaggaggcaggcggccccgatg

SEQ ID NO: 44 - DNA, 104 bp, Artificial sequence
  DLP from Sindbis virus
atgaatagaggattctttaacatgctcggccgccgccccttcccggcccccactgccatgtggaggccgcggagaaggaggcaggcggccccgatgcctgcccg

SEQ ID NO: 45 - DNA, 120 bp, Artificial sequence
  DLP from Aura virus
atgaactctgtcttttacaatccgtttggccgaggtgcctacgctcaacctccaatagcatggaggccaagacgtagggctgcacctgcgcctcgaccatccgggttgactacccagatc

SEQ ID NO: 46 - DNA, 71 bp, Artificial sequence
  DLP from Eastern Equine Encephalitis virus SA
atgtttccgtatccaacattgaactacccgcctatggcaccggttaatccgatggcatacagggaccccaa

SEQ ID NO: 47 - DNA, 91 bp, Artificial sequence
  DLP from O′nyong-nyong virus
atggagttcataccagcacaaacttactacaatagaagataccagcctagaccctggactcaacgccctactatccaggtgatcaggccaa

SEQ ID NO: 48 - DNA, 67 bp, Artificial sequence
  DLP from Semliki forest virus
atgaattacatccctacgcaaacgttttacggccgccggtggcgcccgcgcccggcggcccgtcctt

SEQ ID NO: 49 - DNA, 69 bp, Artificial sequence
  DLP from Ross River virus
atgaattacataccaacccagactttttacggacgccgttggcggcctcgcccggcgttccgtccatgg

SEQ ID NO: 50 - DNA, 91 bp, Artificial sequence
  DLP from Mayaro virus
atggatttcctaccaacacaagtgttttatggcaggcgatggagaccacgaatgccgccacgcccttggaggccacgcccacctacaattc

SEQ ID NO: 51 - protein, 2492 aa, Artificial sequence
  non-structural polyprotein (P1234), PRT, Venezuelan equine encephalitis virus (VEEV)
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGAGSVETPRGLIKVTSYAGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARQFKFSRVCKPKSSLEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQRFDAGAYIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 52 - protein, 2493 aa, Artificial sequence
  non-structural polyprotein (P1234), PRT, eastern equine encephalitis virus (EEEV)
MEKVHVDLDADSPFVKSLQRCFPHFEIEATQVTDNDHANARAFSHLATKLIEGEVDTDQVILDIGSAPVRHTHSKHKYHCICPMKSAEDPDRLYRYADKLRKSDVTDKCIASKAADLLTVMSTPDAETPSLCMHTDSTCRYHGSVAVYQDVYAVHAPTSIYYQALKGVRTIYWIGFDTTPFMYKNMAGAYPTYNTNWADESVLEARNIGLGSSDLHEKSFGKVSIMRKKKLQPTNKVIFSVGSTIYTEERILLRSWHLPNVFHLKGKTSFTGRCNTIVSCEGYVVKKITLSPGIYGKVDNLASTMHREGFLSCKVTDTLRGERVSFPVCTYVPATLCDQMTGILATDVSVDDAQKLLVGLNQRIVVNGRTQRNTNTMQNYLLPVVAQAFSRWAREHRADLEDEKGLGVRERSLVMGCCWAFKTHKITSIYKRPGTQTIKKVPAVFNSFVIPQPTSYGLDIGLRRRIKMLFDAKKAPAPIITEADVAHLKGLQDEAEAVAEAEAVRAALPPLLPEVDKETVEADIDLIMQEAGAGSVETPRRHIKVTTYPGEEMIGSYAVLSPQAVLNSEKLACIHPLAEQVLVMTHKGRAGRYKVEPYHGRVIVPSGTAIPILDFQALSESATIVFNEREFVNRYLHHIAVNGGALNTDEEYYKVVKSTETDSEYVFDIDAKKCVKKGDAGPMCLVGELVDPPFHEFAYESLKTRPAAPHKVPTIGVYGVPGSGKSGIIKSAVTKRDLVVSAKKENCMEIIKDVKRMRGMDIAARTVDSVLLNGVKHSVDTLYIDEAFACHAGTLLALIAIVKPKKVVLCGDPKQCGFFNMMCLKVHFNHEICTEVYHKSISRRCTKTVTSIVSTLFYDKRMRTVNPCNDKIIIDTTSTTKPLKDDIILTCFRGWVKQLQIDYKNHEIMTAAASQGLTRKGVYAVRYKVNENPLYAQTSEHVNVLLTRTEKRIVWKTLAGDPWIKTLTASYPGNFTATLEEWQAEHDAIMAKILETPASSDVFQNKVNVCWAKALEPVLATANITLTRSQWETIPAFKDDKAYSPEMALNFFCTRFFGVDIDSGLFSAPTVPLTYTNEHWDNSPGPNMYGLCMRTAKELARRYPCILKAVDTGRVADVRTDTIKDYNPLINVVPLNRRLPHSLVVTHRYTGNGDYSQLVTKMTGKTVLVVGTPMNIPGKRVETLGPSPQCTYKAELDLGIPAALGKYDIIFINVRTPYRHHHYQQCEDHAIHHSMLTRKAVDHLNKGGTCIALGYGTADRATENIISAVARSFRFSRVCQPKCAWENTEVAFVFFGKDNGNHLQDQDRLSVVLNNIYQGSTQHEAGRAPAYRVVRGDITKSNDEVIVNAANNKGQPGSGVCGALYRKWPGAFDKQPVATGKAHLVKHSPNVIHAVGPNFSRLSENEGDQKLSEVYMDIARIINNERFTKVSIPLLSTGIYAGGKDRVMQSLNHLFTAMDTTDADITIYCLDKQWESRIKEAITRKESVEELTEDDRPVDIELVRVHPLSSLAGRPGYSTTEGKVYSYLEGTRFHQTAKDIAEIYAMWPNKQEANEQICLYVLGESMNSIRSKCPVEESEASSPPHTIPCLCNYAMTAERVYRLRMAKNEQFAVCSSFQLPKYRITGVQKIQCSKPVIFSGTVPPAIHPRKFASVTVEDTPVVQPERLVPRRPAPPVPVPARIPSPPCTSTNGSTTSIQSLGEDQSASASSGAEISVDQVSLWSIPSATGFDVRTSSSLSLEQPTFPTMVVEAEIHASQGSLWSIPSITGSETRAPSPPSQDSRPSTPSASGSHTSVDLITFDSVAEILEDFSRSPFQFLSEIKPIPAPRTRVNNMSRSADTIKPIPKPRKCQVKYTQPPGVARVISAAEFDEFVRRHSNRYEAGAYIFSSETGQGHLQQKSTRQCKLQYPILERSVHEKFYAPRLDLEREKLLQKKLQLCASEGNRSRYQSRKVENMKAITVERLLQGIGSYLSAEPQPVECYKVTYPAPMYSSTASNSFSSAEVAVKVCNLVLQENFPTVASYNITDEYDAYLDMVDGASCCLDTATFCPAKLRSFPKKHSYLRPEIRSAVPSPIQNTLQNVLAAATKRNCNVTQMRELPVLDSAAFNVECFKKYACNDEYWDFYKTNPIRLTAENVTQYVTKLKGPKAAALFAKTHNLQPLHEIPMDRFVMDLKRDVKVTPGTKHTEERPKVQVIQAADPLATAYLCGIHRELVRRLNAVLLPNIHTLFDMSAEDFDAIIAEHFQFGDAVLETDIASFDKSEDDAIAMSALMILEDLGVDQALLNLIEAAFGNITSVHLPTGTRFKFGAMMKSGMFLTLFINTVVNIMIASRVLRERLTTSPCAAFIGDDNIVKGVTSDALMAERCATWLNMEVKIIDAVVGVKAPYFCGGFIVVDQITGTACRVADPLKRLFKLGKPLPLDDDQDVDRRRALHDEAARWNRIGITEELVKAVESRYEVNYVSLIITALTTLASSVSNFKHIRGHPITLYG

SEQ ID NO: 53 - protein, 2431 aa, Artificial sequence
  non-structural polyprotein (P1234), PRT, Semliki forest virus (SFV)
MAAKVHVDIEADSPFIKSLQKAFPSFEVESLQVTPNDHANARAFSHLATKLIEQETDKDTLILDIGSAPSRRMMSTHKYHCVCPMRSAEDPERLDSYAKKLAAASGKVLDREIAGKITDLQTVMATPDAESPTFCLHTDVTCRTAAEVAVYQDVYAVHAPTSLYHQAMKGVRTAYWIGFDTTPFMFDALAGAYPTYATNWADEQVLQARNIGLCAASLTEGRLGKLSILRKKQLKPCDTVMFSVGSTLYTESRKLLRSWHLPSVFHLKGKQSFTCRCDTIVSCEGYVVKKITMCPGLYGKTVGYAVTYHAEGFLVCKTTDTVKGERVSFPVCTYVPSTICDQMTGILATDVTPEDAQKLLVGLNQRIVVNGRTQRNTNTMKNYLLPIVAVAFSKWAREYKADLDDEKPLGVRERSLTCCCLWAFKTRKMHTMYKKPDTQTIVKVPSEFNSFVIPSLWSTGLAIPVRSRIKMLLAKKTKRELIPVLDASSARDAEQEEKERLEAELTREALPPLVPIAPAETGVVDVDVEELEYHAGAGVVETPRSALKVTAQPNDVLLGNYVVLSPQTVLKSSKLAPVHPLAEQVKIITHNGRAGGYQVDGYDGRVLLPCGSAIPVPEFQALSESATMVYNEREFVNRKLYHIAVHGPSLNTDEENYEKVRAERTDAEYVFDVDKKCCVKREEASGLVLVGELTNPPFHEFAYEGLKIRPSAPYKTTVVGVFGVPGSGKSAIIKSLVTKHDLVTSGKKENCQEIVNDVKKHRGKGTSRENSDSILLNGCRRAVDILYVDEAFACHSGTLLALIALVKPRSKVVLCGDPKQCGFFNMMQLKVNFNHNICTEVCHKSISRRCTRPVTAIVSTLHYGGKMRTTNPCNKPIIIDTTGQTKPKPGDIVLTCFRGWAKQLQLDYRGHEVMTAAASQGLTRKGVYAVRQKVNENPLYAPASEHVNVLLTRTEDRLVWKTLAGDPWIKVLSNIPQGNFTATLEEWQEEHDKIMKVIEGPAAPVDAFQNKANVCWAKSLVPVLDTAGIRLTAEEWSTIITAFKEDRAYSPVVALNEICTKYYGVDLDSGLFSAPKVSLYYENNHWDNRPGGRMYGFNAATAARLEARHTFLKGQWHTGKQAVIAERKIQPLSVLDNVIPINRRLPHALVAEYKTVKGSRVEWLVNKVRGYHVLLVSEYNLALPRRRVTWLSPLNVTGADRCYDLSLGLPADAGRFDLVFVNIHTEFRIHHYQQCVDHAMKLQMLGGDALRLLKPGGILMRAYGYADKISEAVVSSLSRKFSSARVLRPDCVTSNTEVFLLFSNFDNGKRPSTLHQMNTKLSAVYAGEAMHTAGCAPSYRVKRADIATCTEAAVVNAANARGTVGDGVCRAVAKKWPSAFKGAATPVGTIKTVMCGSYPVIHAVAPNFSATTEAEGDRELAAVYRAVAAEVNRLSLSSVAIPLLSTGVFSGGRDRLQQSLNHLFTAMDATDADVTIYCRDKSWEKKIQEAIDMRTAVELLNDDVELTTDLVRVHPDSSLVGRKGYSTTDGSLYSYFEGTKFNQAAIDMAEILTLWPRLQEANERICLYALGETMDNIGSKCPVNDSDSSTPPRTVPCLCRYAMTAERIARLRSHQVKSMVVCSSFPLPKYHVDGVQKVKCEKVLLFDPTVPSVVSPRKYAASTTDHSDRSLRGFDLDWTTDSSSTASDTMSLPSLQSCDIDSIYEPMAPIVVTADVHPEPAGIADLAADVHPEPADHVDLENPIPPPRPKRAAYLASRAAERPVPAPRKPTPAPRTAFRNKLPLTFGDFDEHEVDALASGITFGDFDDVLRLGRAGAYIFSSDTGSGHLQQKSVRQHNLQCAQLDAVQEEKMYPPKLDTEREKLLLLKMQMHPSEANKSRYQSRKVENMKATVVDRLTSGARLYTGADVGRIPTYAVRYPRPVYSPTVIERFSSPDVAIAACNEYLSRNYPTVASYQITDEYDAYLDMVDGSDSCLDRATFCPAKLRCYPKHHAYHQPTVRSAVPSPFQNTLQNVLAAATKRNCNVTQMRELPTMDSAVFNVECFKRYACSGEYWEEYAKQPIRITTENITTYVTKLKGPKAAALFAKTHNLVPLQEVPMDRFTVDMKRDVKVTPGTKHTEERPKVQVIQAAEPLATAYLCGIHRELVRRLNAVLRPNVHTLFDMSAEDFDAIIASHFHPGDPVLETDIASFDKSQDDSLALTGLMILEDLGVDQYLLDLIEAAFGEISSCHLPTGTRFKFGAMMKSGMFLTLFINTVLNITIASRVLEQRLTDSACAAFIGDDNIVHGVISDKLMAERCASWVNMEVKIIDAVMGEKPPYFCGGFIVFDSVTQTACRVSDPLKRLFKLGKPLTAEDKQDEDRRRALSDEVSKWFRTGLGAELEVALTSRYEVEGCKSILIAMTTLARDIKAFKKLRGPVIHLYGGPRLVR

SEQ ID NO: 54 - protein, 2512 aa, Artificial sequence
  non-structural polyprotein (P1234), PRT, Sindbis virus (SINV)
MEKPVVNVDVDPQSPFVVQLQKSFPQFEVVAQQVTPNDHANARAFSHLASKLIELEVPTTATILDIGSAPARRMFSEHQYHCVCPMRSPEDPDRMMKYASKLAEKACKITNKNLHEKIKDLRTVLDTPDAETPSLCFHNDVTCNMRAEYSVMQDVYINAPGTIYHQAMKGVRTLYWIGFDTTQFMFSAMAGSYPAYNTNWADEKVLEARNIGLCSTKLSEGRTGKLSIMRKKELKPGSRVYFSVGSTLYPEHRASLQSWHLPSVFHLNGKQSYTCRCDTVVSCEGYVVKKITISPGITGETVGYAVTHNSEGFLLCKVTDTVKGERVSFPVCTYIPATICDQMTGIMATDISPDDAQKLLVGLNQRIVINGRTNRNTNTMQNYLLPIIAQGFSKWAKERKDDLDNEKMLGTRERKLTYGCLWAFRTKKVHSFYRPPGTQTCVKVPASFSAFPMSSVWTTSLPMSLRQKLKLALQPKKEEKLLQVSEELVMEAKAAFEDAQEEARAEKLREALPPLVADKGIEAAAEVVCEVEGLQADIGAALVETPRGHVRIIPQANDRMIGQYIVVSPNSVLKNAKLAPAHPLADQVKIITHSGRSGRYAVEPYDAKVLMPAGGAVPWPEFLALSESATLVYNEREFVNRKLYHIAMHGPAKNTEEEQYKVTKAELAETEYVFDVDKKRCVKKEEASGLVLSGELTNPPYHELALEGLKTRPAVPYKVETIGVIGTPGSGKSAIIKSTVTARDLVTSGKKENCREIEADVLRLRGMQITSKTVDSVMLNGCHKAVEVLYVDEAFACHAGALLALIAIVRPRKKVVLCGDPMQCGFFNMMQLKVHFNHPEKDICTKTFYKYISRRCTQPVTAIVSTLHYDGKMKTTNPCKKNIEIDITGATKPKPGDIILTCFRGWVKQLQIDYPGHEVMTAAASQGLTRKGVYAVRQKVNENPLYAITSEHVNVLLTRTEDRLVWKTLQGDPWIKQPTNIPKGNFQATIEDWEAEHKGIIAAINSPTPRANPFSCKTNVCWAKALEPILATAGIVLTGCQWSELFPQFADDKPHSAIYALDVICIKFFGMDLTSGLFSKQSIPLTYHPADSARPVAHWDNSPGTRKYGYDHAIAAELSRRFPVFQLAGKGTQLDLQTGRTRVISAQHNLVPVNRNLPHALVPEYKEKQPGPVKKFLNQFKHHSVLVVSEEKIEAPRKRIEWIAPIGIAGADKNYNLAFGFPPQARYDLVFINIGTKYRNHHFQQCEDHAATLKTLSRSALNCLNPGGTLVVKSYGYADRNSEDVVTALARKFVRVSAARPDCVSSNTEMYLIFRQLDNSRTRQFTPHHLNCVISSVYEGTRDGVGAAPSYRTKRENIADCQEEAVVNAANPLGRPGEGVCRAIYKRWPTSFTDSATETGTARMTVCLGKKVIHAVGPDFRKHPEAEALKLLQNAYHAVADLVNEHNIKSVAIPLLSTGIYAAGKDRLEVSLNCLTTALDRTDADVTIYCLDKKWKERIDAALQLKESVTELKDEDMEIDDELVWIHPDSCLKGRKGFSTTKGKLYSYFEGTKFHQAAKDMAEIKVLFPNDQESNEQLCAYILGETMEAIREKCPVDHNPSSSPPKTLPCLCMYAMTPERVHRLRSNNVKEVTVCSSTPLPKHKIKNVQKVQCTKVVLFNPHTPAFVPARKYIEVPEQPTAPPAQAEEAPEVVATPSPSTADNTSLDVTDISLDMDDSSEGSLFSSFSGSDNSITSMDSWSSGPSSLEIVDRRQVVVADVHAVQEPAPIPPPRLKKMARLAAARKEPTPPASNSSESLHLSFGGVSMSLGSIFDGETARQAAVQPLATGPTDVPMSFGSFSDGEIDELSRRVTESEPVLFGSFEPGEVNSIISSRSAVSFPLRKQRRRRRSRRTEYLTGVGGYIFSTDTGPGHLQKKSVLQNQLTEPTLERNVLERIHAPVLDTSKEEQLKLRYQMMPTEANKSRYQSRKVENQKAITTERLLSGLRLYNSATDQPECYKITYPKPLYSSSVPANYSDPQFAVAVCNNYLHENYPTVASYQITDEYDAYLDMVDGTVACLDTATFCPAKLRSYPKKHEYRAPNIRSAVPSAMQNTLQNVLIAATKRNCNVTQMRELPTLDSATFNVECFRKYACNDEYWEEFARKPIRITTEFVTAYVARLKGPKAAALFAKTYNLVPLQEVPMDRFVMDMKRDVKVTPGTKHTEERPKVQVIQAAEPLATAYLCGIHRELVRRLTAVLLPNIHTLFDMSAEDFDAIIAEHFKQGDPVLETDIASFDKSQDDAMALTGLMILEDLGVDQPLLDLIECAFGEISSTHLPTGTRFKFGAMMKSGMFLTLFVNTVLNVVIASRVLEERLKTSRCAAFIGDDNIIHGVVSDKEMAERCATWLNMEVKIIDAVIGERPPYFCGGFILQDSVTSTACRVADPLKRLFKLGKPLPADDEQDEDRRRALLDETKAWFRVGITGTLAVAVTTRYEVDNITPVLLALRTFAQSKRAFQAIRGEIKHLYGGPK

SEQ ID NO: 55 - protein, 2474 aa, Artificial sequence
  non-structural polyprotein (P1234), PRT, Chikungunya virus (CHIKV)
MDPVYVDIDADSAFLKALQRAYPMFEVEPRQVTPNDHANARAFSHLAIKLIEQEIDPDSTILDIGSAPARRMMSDRKYHCVCPMRSAEDPERLANYARKLASAAGKVLDRNISGKIGDLQAVMAVPDTETPTFCLHTDVSCRQRADVAIYQDVYAVHAPTSLYHQAIKGVRLAYWVGFDTTPFMYNAMAGAYPSYSTNWADEQVLKAKNIGLCSTDLTEGRRGKLSIMRGKKLEPCDRVLFSVGSTLYPESRKLLKSWHLPSVFHLKGKLSFTCRCDTVVSCEGYVVKRITMSPGLYGKTTGYAVTHHADGFLMCKTTDTVDGERVSFSVCTYVPATICDQMTGILATEVTPEDAQKLLVGLNQRIVVNGRTQRNTNTMKNYMIPVVAQAFSKWAKECRKDMEDEKLLGVRERTLTCCCLWAFKKQKTHTVYKRPDTQSIQKVQAEFDSFVVPSLWSSGLSIPLRTRIKWLLSKVPKTDLTPYSGDAQEARDAEKEAEEEREAELTLEALPPLQAAQEDVQVEIDVEQLEDRAGAGIIETPRGAIKVTAQPTDHVVGEYLVLSPQTVLRSQKLSLIHALAEQVKTCTHSGRAGRYAVEAYDGRVLVPSGYAISPEDFQSLSESATMVYNEREFVNRKLHHIAMHGPALNTDEESYELVRAERTEHEYVYDVDQRRCCKKEEAAGLVLVGDLTNPPYHEFAYEGLKIRPACPYKIAVIGVFGVPGSGKSAIIKNLVTRQDLVTSGKKENCQEITTDVMRQRGLEISARTVDSLLLNGCNRPVDVLYVDEAFACHSGTLLALIALVRPRQKVVLCGDPKQCGFFNMMQMKVNYNHNICTQVYHKSISRRCTLPVTAIVSSLHYEGKMRTTNEYNKPIVVDTTGSTKPDPGDLVLTCFRGWVKQLQIDYRGHEVMTAAASQGLTRKGVYAVRQKVNENPLYASTSEHVNVLLTRTEGKLVWKTLSGDPWIKTLQNPPKGNFKATIKEWEVEHASIMAGICSHQMTFDTFQNKANVCWAKSLVPILETAGIKLNDRQWSQIIQAFKEDKAYSPEVALNEICTRMYGVDLDSGLFSKPLVSVYYADNHWDNRPGGKMFGFNPEAASILERKYPFTKGKWNINKQICVTTRRIEDFNPTTNIIPANRRLPHSLVAEHRPVKGERMEWLVNKINGHHVLLVSGCSLALPTKRVTWVAPLGVRGADYTYNLELGLPATLGRYDLVVINIHTPFRIHHYQQCVDHAMKLQMLGGDSLRLLKPGGSLLIRAYGYADRTSERVICVLGRKFRSSRALKPPCVTSNTEMFFLFSNFDNGRRNFTTHVMNNQLNAAFVGQATRAGCAPSYRVKRMDIAKNDEECVVNAANPRGLPGDGVCKAVYKKWPESFKNSATPVGTAKTVMCGTYPVIHAVGPNFSNYSESEGDRELAAAYREVAKEVTRLGVNSVAIPLLSTGVYSGGKDRLTQSLNHLFTAMDSTDADVVIYCRDKEWEKKISEAIQMRTQVELLDEHISIDCDVVRVHPDSSLAGRKGYSTTEGALYSYLEGTRFHQTAVDMAEIYTMWPKQTEANEQVCLYALGESIESIRQKCPVDDADASSPPKTVPCLCRYAMTPERVTRLRMNHVTSIIVCSSFPLPKYKIEGVQKVKCSKVMLFDHNVPSRVSPREYRPSQESVQEASTTTSLTHSQFDLSVDGKILPVPSDLDADAPALEPALDDGAIHTLPSATGNLAAVSDWVMSTVPVAPPRRRRGRNLTVTCDEREGNITPMASVRFFRAELCPVVQETAETRDTAMSLQAPPSTATELSHPPISFGAPSETFPITFGDFNEGEIESLSSELLTFGDFLPGEVDDLTDSDWSTCSDTDDELRLDRAGGYIFSSDTGPGHLQQKSVRQSVLPVNTLEEVHEEKCYPPKLDEAKEQLLLKKLQESASMANRSRYQSRKVENMKATIIQRLKRGCRLYLMSETPKVPTYRTTYPAPVYSPPINVRLSNPESAVAACNEFLARNYPTVSSYQITDEYDAYLDMVDGSESCLDRATFNPSKLRSYPKQHAYHAPSIRSAVPSPFQNTLQNVLAAATKRNCNVTQMRELPTLDSAVFNVECFKKFACNQEYWEEFAASPIRITTENLTTYVTKLKGPKAAALFAKTHNLLPLQEVPMDRFTVDMKRDVKVTPGTKHTEERPKVQVIQAAEPLATAYLCGIHRELVRRLNAVLLPNVHTLFDMSAEDFDAIIAAHFKPGDTVLETDIASFDKSQDDSLALTALMLLEDLGVDHSLLDLIEAAFGEISSCHLPTGTRFKFGAMMKSGMFLTLFVNTLLNITIASRVLEDRLTKSACAAFIGDDNIIHGVVSDELMAARCATWMNMEVKIIDAVVSQKAPYFCGGFILHDIVTGTACRVADPLKRLFKLGKPLAAGDEQDEDRRRALADEVVRWQRTGLIDELEKAVYSRYEVQGISVVVMSMATFASSRSNFEKLRGPVVTLYGGPK

SEQ ID NO: 56 - protein, 14 aa, Artificial sequence
  483-495 of CHIKV, PRT, chikungunya virus
NEGEIESLSSELLT

SEQ ID NO: 57 - protein, 20 aa, Artificial sequence
  494-512 of SINV, PRT, sindbis virus
SDGEIDELSRRVTTESEPVL

SEQ ID NO: 58 - protein, 13 aa, Artificial sequence
  448-459 of SFV, PRT, semliki forest virus
DEHEVDALASGIT

SEQ ID NO: 59 - protein, 30 aa, Artificial sequence
  501-530 or CHIKV, PRT, chikungunya virus
LPGEVDDLTDSDWSTCSDTDDELRLDRAGG

SEQ ID NO: 60 - protein, 33 aa, Artificial sequence
  517-549 of SINV, PRT, sindbis virus
EPGEVNSIISSRSAVSFPLRKQRRRRRSRRTEY

SEQ ID NO: 61 - protein, 11 aa, Artificial sequence
  465-475 of SFV, PRT, semliki forest virus
DDVLRLGRAGA

SEQ ID NO: 62 - protein, 10 aa, Artificial sequence
  nsP2/nsP3 junction, PRT, venezuelan equine encephalitis virus
LHEAGCAPSY

SEQ ID NO: 63 - protein, 10 aa, Artificial sequence
  nsP3/nsP4 junction, PRT, venezuelan equine encephalitis virus
RFDAGAYIFS

SEQ ID NO: 64 - protein, 10 aa, Artificial sequence
  nsP2/nsP3 junction, PRT, eastern equine encephalitis virus
QHEAGRAPAY

SEQ ID NO: 65 - protein, 10 aa, Artificial sequence
  nsP3/nsP4 junction, PRT, eastern equine encephalitis virus
RYEAGAYIFS

SEQ ID NO: 66 - protein, 10 aa, Artificial sequence
  nsP2/nsP3 junction, PRT, western equine encephalitis virus
RYEAGRAPAY

SEQ ID NO: 67 - protein, 10 aa, Artificial sequence
  nsP3/nsP4 junction, PRT, western equine encephalitis virus
RYEAGAYIFS

(19) United States
(12) Patent Application Publication
(10) Pub. No.: US 2022/0313815 A1
(43) Pub. Date: Oct. 6, 2022

(54) SELF-REPLICATING RNA MOLECULES FOR HEPATITIS B VIRUS (HBV) VACCINES AND USES THEREOF

(71) Applicant: Janssen Sciences Ireland Unlimited Company, Co Cork (IE)

(72) Inventors: Helen HORTON, Beerse (BE); Daniel BODEN, Beerse (BE)

(21) Appl. No.: 17/596,874

(22) PCT Filed: Jun. 19, 2020
(86) PCT No.: PCT/IB2020/055775

§ 371 Date: Dec. 20, 2021

Related U.S. Application Data
(60) Provisional application No. 62/863,961, filed on Jun. 20, 2019.
(60) Provisional application No. 63/006,925, filed on Apr. 8, 2020.

(51) Int. Cl. A61K 39/29; A61P 31/20

(52) U.S. Cl. CPC A61K 39/292; A61P 31/20; A61K 2039/5256

(57) ABSTRACT

Self-replicating RNA molecules encoding hepatitis B virus (HBV) vaccines are described. Methods of inducing an immune response against HBV or treating an HBV-induced disease, particularly in individuals having chronic HBV infection, using the disclosed self-replicating RNA molecules are also described. Kits comprising the disclosed self-replicating RNA molecules are also described.

### CROSS REFERENCE TO RELATED APPLICATIONS

This application claims priority to U.S. Provisional No. 63/006,925, filed on Apr. 8, 2020, and U.S. Provisional No. 62/863,961, filed on Jun. 20, 2019, the disclosures of each of which are incorporated by reference in their entireties.

### REFERENCE TO SEQUENCE LISTING SUBMITTED ELECTRONICALLY

This application contains a sequence listing, which is submitted electronically via EFS-Web as an ASCII formatted sequence listing with a file name “065814.11217_9WO1 Sequence Listing” with a creation date of Jun. 11, 2020 and having a size of 172 kb. The sequence listing submitted via EFS-Web is part of the specification and is herein incorporated by reference in its entirety.

### BACKGROUND OF THE INVENTION

Hepatitis B virus (HBV) is a small 3.2-kb hepatotropic DNA virus that encodes four open reading frames and seven proteins. Approximately 240 million people have chronic hepatitis B infection (chronic HBV), characterized by persistent virus and subvirus particles in the blood for more than 6 months (Cohen et al. J. Viral Hepat. (2011) 18(6), 377-83). Persistent HBV infection leads to T-cell exhaustion in circulating and intrahepatic HBV-specific CD4+ and CD8+ T-cells through chronic stimulation of HBV-specific T-cell receptors with viral peptides and circulating antigens. As a result, T-cell polyfunctionality is decreased (i.e., decreased levels of IL-2, tumor necrosis factor (TNF)-α, IFN-γ, and lack of proliferation).

A safe and effective prophylactic vaccine against HBV infection has been available since the 1980s and is the mainstay of hepatitis B prevention (World Health Organization, Hepatitis B: Fact sheet No. 204 [Internet] 2015 March.). The World Health Organization recommends vaccination of all infants, and, in countries where there is low or intermediate hepatitis B endemicity, vaccination of all children and adolescents (<18 years of age), and of people of certain at risk population categories. Due to vaccination, worldwide infection rates have dropped dramatically. However, prophylactic vaccines do not cure established HBV infection.

Chronic HBV is currently treated with IFN-a and nucleoside or nucleotide analogs, but there is no ultimate cure due to the persistence in infected hepatocytes of an intracellular viral replication intermediate called covalently closed circular DNA (cccDNA), which plays a fundamental role as a template for viral RNAs, and thus new virions. It is thought that induced virus-specific T-cell and B-cell responses can effectively eliminate cccDNA-carrying hepatocytes. Current therapies targeting the HBV polymerase suppress viremia, but offer limited effect on cccDNA that resides in the nucleus and related production of circulating antigen. The most rigorous form of a cure can be elimination of HBV cccDNA from the organism, which has neither been observed as a naturally occurring outcome nor as a result of any therapeutic intervention. However, loss of HBV surface antigens (HBsAg) is a clinically credible equivalent of a cure, since disease relapse can occur only in cases of severe immunosuppression, which can then be prevented by prophylactic treatment. Thus, at least from a clinical standpoint, loss of HBsAg is associated with the most stringent form of immune reconstitution against HBV.

For example, immune modulation with pegylated interferon (pegIFN)-α has proven better in comparison to nucleoside or nucleotide therapy in terms of sustained off-treatment response with a finite treatment course. Besides a direct antiviral effect, IFN-α is reported to exert epigenetic suppression of cccDNA in cell culture and humanized mice, which leads to reduction of virion productivity and transcripts (Belloni et al. J. Clin. Invest. (2012) 122(2), 529-537). However, this therapy is still fraught with side-effects and overall responses are rather low, in part because IFN-α has only poor modulatory influences on HBV-specific T-cells. In particular, cure rates are low (<10%) and toxicity is high. Likewise, direct acting HBV antivirals, namely the HBV polymerase inhibitors entecavir and tenofovir, are effective as monotherapy in inducing viral suppression with a high genetic barrier to emergence of drug resistant mutants and consecutive prevention of liver disease progression. However, cure of chronic hepatitis B, defined by HBsAg loss or seroconversion, is rarely achieved with such HBV polymerase inhibitors. Therefore, these antivirals in theory need to be administered indefinitely to prevent reoccurrence of liver disease, similar to antiretroviral therapy for human immunodeficiency virus (HIV).

Therapeutic vaccination has the potential to eliminate HBV from chronically infected patients (Michel et al. J. Hepatol. (2011) 54(6), 1286-1296). Many strategies have been explored, but to date therapeutic vaccination has not proven successful.

### BRIEF SUMMARY OF THE INVENTION

Accordingly, there is an unmet medical need in the treatment of hepatitis B virus (HBV), particularly chronic HBV, for a finite well-tolerated treatment with a higher cure rate. The invention satisfies this need by providing therapeutic compositions and methods for inducing an immune response against hepatitis B viruses (HBV) infection. The immunogenic compositions/combinations and methods of the invention can be used to provide therapeutic immunity to a subject, such as a subject having chronic HBV infection.

In a general aspect, the application relates to a self-replicating RNA molecule comprising one or more polynucleotides encoding HBV antigens for use in treating an HBV infection in a subject in need thereof.

In one embodiment, the self-replicating RNA molecule comprises at least one of:
a) a first polynucleotide sequence encoding a truncated HBV core antigen consisting of an amino acid sequence that is at least 95%, 96%, 97%, 98%, 99% or 100% identical to SEQ ID NO: 2; or
b) a second polynucleotide sequence encoding the HBV polymerase antigen consisting of an amino acid sequence that is at least 90%, such as at least 90%, 91%, 92%, 93%, 94%, 95%, 96%, 97%, 98%, 99% or 100% identical to SEQ ID NO: 7, wherein the HBV polymerase antigen does not have reverse transcriptase activity and RNase H activity.

In one embodiment, the self-replicating RNA molecule comprises the first polynucleotide sequence encoding a truncated HBV core antigen consisting of an amino acid sequence that is at least 95% identical to SEQ ID NO: 2. In another embodiment, the self-replicating RNA molecule comprises the second polynucleotide encoding the HBV polymerase antigen consisting of an amino acid sequence that is at least 90% identical to SEQ ID NO: 7.

In an embodiment, a self-replicating RNA molecule comprises:
a) a first polynucleotide sequence encoding a truncated HBV core antigen consisting of an amino acid sequence that is at least 95%, 96%, 97%, 98%, 99% or 100% identical to SEQ ID NO: 2; and
b) a second polynucleotide sequence encoding the HBV polymerase antigen consisting of an amino acid sequence that is at least 90%, 91%, 92%, 93%, 94%, 95%, 96%, 97%, 98%, 99% or 100% identical to SEQ ID NO: 7, wherein the HBV polymerase antigen does not have reverse transcriptase activity and RNase H activity.

In certain embodiments, the first polynucleotide sequence further comprises a polynucleotide sequence encoding a signal sequence operably linked to the N-terminus of the truncated HBV core antigen, and the second polynucleotide sequence further comprises a polynucleotide sequence encoding a signal sequence operably linked to the N-terminus of the HBV polymerase antigen, preferably, the signal sequence independently comprises the amino acid sequence of SEQ ID NO: 9 or SEQ ID NO: 15, preferably the signal sequence is independently encoded by the polynucleotide sequence of SEQ ID NO: 8 or SEQ ID NO: 14, respectively.

In certain embodiments, the first polynucleotide sequence encoding a truncated HBV core antigen consists of an amino acid sequence of SEQ ID NO: 2; and the second polynucleotide sequence encoding the HBV polymerase antigen consists of an amino acid sequence of SEQ ID NO: 7. Preferably, the self-replicating RNA molecule comprises a) a first polynucleotide sequence encoding an truncated HBV core antigen consisting of the amino acid sequence of SEQ ID NO: 2 or SEQ ID NO: 4; and b) a second polynucleotide sequence encoding an HBV polymerase antigen having the amino acid sequence of SEQ ID NO: 7.

In certain embodiments, the first polynucleotide sequence comprises the polynucleotide sequence having at least 90%, such as at least 90%, 91%, 92%, 93%, 94%, 95%, 96%, 97%, 98%, 99% or 100%, sequence identity to SEQ ID NO: 1 or SEQ ID NO: 3.

In certain embodiments, the second polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 5 or SEQ ID NO: 6.

In an embodiment, the self-replicating RNA molecule encodes a fusion protein comprising the truncated HBV core antigen operably linked to the HBV polymerase antigen. In certain embodiments, the fusion protein comprises the truncated HBV core antigen operably linked to the HBV polymerase antigen via a linker. Preferably, the linker comprises the amino acid sequence of (AlaGly)n, and n is an integer of 2 to 5, preferably the linker is encoded by a polynucleotide sequence comprising SEQ ID NO: 11. Preferably, the fusion protein comprises the amino acid sequence of SEQ ID NO: 16.

In certain embodiments, the self-replicating RNA molecule is an alphavirus-derived RNA replicon. In certain embodiments, the RNA replicon comprises one or more alphavirus non-structural protein genes. In certain embodiments, the RNA replicon comprises genetic elements required for RNA replication and lacks those genetic elements encoding gene products necessary for viral particle assembly, and the RNA replicon is delivered to a subject in a composition containing no viral protein, such as in a lipid composition (e.g., a lipid nanoparticle) or another suitable composition. In other embodiments, the RNA replicon comprises genetic elements required for RNA replication and those genetic elements encoding gene products necessary for viral particle assembly, and the RNA replicon is delivered to a subject in a composition containing one or more viral proteins, such as a viral like particle. In further embodiments, the RNA replicon comprises one or more modifications that enhance gene expression and/or confer a resistance to the innate immune system, such as stem-loops or downstream loops (a DLP motif) that enhance the translation of RNA under the control of a subgenomic promoter (Fovlov et al., J Virol. 1996, 70:1182-90).

In certain embodiments, examples of self-replicating RNA molecules, compositions and methods to create and use such molecules for delivering genes of interest are described in U.S. Patent Application Publications US2018/0104359, US2013/0177639, US2013/0149375, US 2014/0242152, International Patent Application Publication WO2018/075235 or U.S. Pat. No. 10,022,435, the contents of which are incorporated herein by reference in their entireties. For example, the RNA replications can include one or more components such as a 5′ UTR, a viral capsid enhancer Downstream Loop (DLP), and an Old World alphavirus nsP3 hypervariable domain or a chimeric nsP3 hypervariable domain containing a portion of a New World alphavirus nsP3 hypervariable domain and another portion derived from an Old World alphavirus nsP3 hypervariable domain, as described in U.S. Patent Application Publications US2018/0104359, US2018/0171340, and U.S. Patent Application No. 62/742,868, respectively, each of which is incorporated herein by reference in its entirety.

In certain embodiments, a self-replicating RNA molecule comprises:
a) one or more nonstructural genes nsP1, nsP2, nsP3 and nsP4;
b) at least one of a downstream loop (DLP motif) and a modified 5′-untranslated region (5′-UTR);
c) a subgenomic promoter; and
d) at least one of
   i. a first polynucleotide sequence encoding a truncated HBV core antigen consisting of an amino acid sequence that is at least 95% identical to SEQ ID NO: 2; or
   ii. a second polynucleotide sequence encoding the HBV polymerase antigen consisting of an amino acid sequence that is at least 90% identical to SEQ ID NO: 7, wherein the HBV polymerase antigen does not have reverse transcriptase activity and RNase H activity;
operably linked to the subgenomic promoter.

The RNA replicons are useful for the administration of biotherapeutic molecules such as proteins and peptides, where the replicons of the invention are administered to a human or animal with the biotherapeutic being encoded by the replicon, and the encoded biotherapeutic (e.g. a heterologous protein or peptide) is expressed in the human or animal.

In one aspect, disclosed herein is a nucleic acid molecule including a modified replicon RNA encoding an HBV antigen described herein, in which the modified replicon RNA includes a modified 5-'UTR and is devoid of at least a portion of a nucleic acid sequence encoding viral structural proteins. In some embodiments, the modified 5′-UTR includes one or more nucleotide substitutions at position 1, 2, 4, or a combination thereof. In some embodiments, at least one of the nucleotide substitutions is a nucleotide substitution at position 2 of the modified 5′-UTR. In some embodiments, the nucleotide substitutions at position 2 of the modified 5′-UTR is a U->G substitution.

In some embodiments, the nucleic acid molecule as disclosed herein includes a modified alphavirus genome or replicon RNA including a modified alphavirus genome or replicon RNA, wherein the nucleic acid molecule comprises a nucleotide sequence exhibiting at least 80% sequence identity to the nucleic acid sequence of SEQ ID NO: 1, the modified alphavirus genome or replicon RNA comprises a U->G substitution at position 2 of the 5′-untranslated region (5′-UTR) and is devoid of at least a portion of the sequence encoding viral structural proteins. In some embodiments, the nucleic acid molecule comprises a nucleotide sequence exhibiting at least 90%, at least 95%, at least 96%, at least 97%, at least 98%, at least 99%, or 100% sequence identity to the nucleic acid sequence of SEQ ID NO: 25.

In some embodiments, the nucleic acid molecule as disclosed herein includes a modified alphavirus genome or replicon RNA, wherein the modified alphavirus genome or replicon RNA comprises a 5′-UTR exhibiting at least 80% sequence identity to the nucleic acid sequence of at least one of SEQ ID NOs: 26-42 and a U->G substitution at position 2 of the 5′-UTR, and wherein the modified alphavirus genome or replicon RNA is devoid of at least a portion of the sequence encoding viral structural proteins. In some embodiments, the modified alphavirus genome or replicon RNA comprises a 5′-UTR exhibiting at least 90%, at least 95%, at least 96%, at least 97%, at least 98%, at least 99%, or 100% sequence identity to the nucleic acid sequence of at least one of SEQ ID NOs: 26-42. In certain embodiments, the modified alphavirus genome or replicon RNA is devoid of a substantial portion of the nucleic acid sequence encoding viral structural proteins. In certain embodiments, the modified alphavirus genome or replicon RNA comprises no nucleic acid sequence encoding viral structural proteins.

Implementations of embodiments of the methods according to the present disclosure can include one or more of the following features. In some embodiments, the modified replicon RNA is a modified alphavirus replicon RNA. In some embodiments, the modified alphavirus replicon RNA includes a modified alphavirus genome. In some embodiments, the modified 5′-UTR includes one or more nucleotide substitutions at position 1, 2, 4, or a combination thereof. In some embodiments, at least one of the nucleotide substitutions is a nucleotide substitution at position 2 of the modified 5′-UTR. In some embodiments, the nucleotide substitutions at position 2 of the modified 5′-UTR is a U->G substitution. In certain embodiments, the modified replicon RNA is devoid of a substantial portion of the nucleic acid sequence encoding viral structural proteins. In some embodiments, the modified alphavirus genome or replicon RNA includes no nucleic acid sequence encoding viral structural proteins.

In some embodiments, the nucleic acid molecule includes a modified alphavirus genome or replicon RNA, wherein the modified alphavirus genome or replicon RNA includes a 5′-UTR exhibiting at least 80% sequence identity to the nucleic acid sequence of SEQ ID NO: 25 and a U->G substitution at position 2 of the 5′-UTR, and wherein the modified alphavirus genome or replicon RNA is devoid of at least a portion of the sequence encoding viral structural proteins. In some embodiments, the nucleic acid molecule exhibits at least 90%, at least 95%, at least 96%, at least 97%, at least 98%, at least 99%, or 100% sequence identity to the nucleic acid sequence of SEQ ID NO: 25. In some embodiments, the nucleic acid molecule includes a modified alphavirus genome or replicon RNA, wherein the modified alphavirus genome or replicon RNA includes a 5′-UTR exhibiting at least 80% sequence identity to the nucleic acid sequence of at least one of SEQ ID NOs: 26-42 and a U->G substitution at position 2 of the 5′-UTR, and wherein the modified alphavirus genome or replicon RNA is devoid of at least a portion of the sequence encoding viral structural proteins. In some embodiments, the modified alphavirus genome or replicon RNA includes a 5′-UTR exhibiting at least 90%, at least 95%, at least 96%, at least 97%, at least 98%, at least 99%, or 100% sequence identity to the nucleic acid sequence of at least one of SEQ ID NOs: 26-42.

In one aspect, some embodiments disclosed herein relate to a nucleic acid molecule, including (i) a first nucleic acid sequence encoding one or more RNA stem-loops of a viral capsid enhancer (FIG. 6) or a variant thereof; and (ii) a second nucleic acid sequence operably linked to the first nucleic acid sequence, wherein the second nucleic acid sequence comprises a coding sequence for a gene of interest (GOI) encoding the HBV core and/or the HBV polymerase of the invention.

Implementations of embodiments of the nucleic acid molecule according to the present disclosure can include one or more of the following features. In some embodiments, the first nucleic acid sequence is operably linked upstream to the coding sequence for the GOI (e.g., the one or more HBV antigens described herein). In some embodiments, the nucleic acid molecule further includes a promoter operably linked upstream to the first nucleic acid sequence. In some embodiments, the nucleic acid molecule further includes a 5′ UTR sequence operably linked upstream to the first nucleic acid sequence. In some embodiments, the 5′ UTR sequence is operably linked downstream to the promoter and upstream to the first nucleic acid sequence. In some embodiments, the nucleic acid molecule further includes a coding sequence for an autoprotease peptide operably linked upstream to the second nucleic acid sequence. In some embodiments, the coding sequence for the autoprotease peptide is operably linked downstream to the first nucleic acid sequence and upstream to the second nucleic acid sequence.

In some embodiments, the autoprotease peptide comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In some embodiments, the nucleic acid molecule further includes a 3′ UTR sequence operably linked downstream to the second sequence nucleic acid sequence.

In some embodiments, the viral capsid enhancer is derived from a capsid gene of a virus species belonging to the Togaviridae family. In some embodiments, the alphavirus species is Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABY), Kyzylagach virus (KYZV), Western equine encephalitis virus (WEEV), Highland J virus (HJV), Fort Morgan virus (FMV), Ndumu (NDUV), Salmonid alphavirus (SAV), or Buggy Creek virus. In some embodiments, the viral capsid enhancer comprises a downstream loop (DLP) motif of the virus species, and the DLP motif comprises one or more RNA stem-loops. In some embodiments, the viral capsid enhancer comprises a nucleic acid sequence exhibiting at least 80% sequence identity to at least one of SEQ ID NOs: 43-50. In some embodiments, the nucleic acid sequence exhibits at least 95% sequence identity to at least one of SEQ ID NOs: 43-50.

In some embodiments, the nucleic acid molecule of the disclosure further includes a third nucleic acid sequence encoding one or more RNA stem-loops of a second viral capsid enhancer or a variant thereof and a fourth nucleic acid sequence operably linked to the third nucleic acid sequence, wherein the fourth nucleic acid sequence comprises a coding sequence for a second gene of interest (GOI). In some embodiments, the nucleic acid molecule further includes a coding sequence for a second autoprotease peptide operably linked downstream to the third nucleic acid sequence and upstream to the fourth nucleic acid sequence.

In certain embodiments, the self-replicating RNA molecule contains New World alphavirus nonstructural proteins nsP1, nsP2, and nsP4; and an alphavirus nsP3 protein macro domain, central domain, and hypervariable domain. The encoded hypervariable domain can have an amino acid sequence derived from an Old World alphavirus nsP3 hypervariable domain, or can have an amino acid sequence derived from a portion of a New World alphavirus nsP3 hypervariable domain, and another portion derived from an Old World alphavirus nsP3 hypervariable domain, i.e. a chimeric nsP3 hypervariable domain. It was found that when the replicon based on a New World alphavirus is modified, an immune response provoked by the encoded heterologous protein or peptide, such as the at least one of HBV core and polymerase antigens, is diminished or eliminated.

In one embodiment, the alphavirus nsP3 macro domain and the alphavirus nsP3 central domain are from a New World alphavirus, but in another embodiment, the alphavirus nsP3 macro domain and the alphavirus nsP3 central domain are from an Old World alphavirus. In various embodiments the Old World alphavirus is selected from the group consisting of: CHIKV, SINV, and SFV. The New World alphavirus can be Venezuelan Equine Encephalitis Virus (VEEV) or western equine encephalitis virus (WEEV), or eastern equine encephalitis virus (EEEV). In various embodiments the Old World alphavirus can be any of Sindbis virus (SINV), Chickungunya virus (CHIKV), Semliki Forest Virus (SFV), Ross River Virus (RRV), Sagiyama virus (SAGV), Getah virus (GETV), Middleburg virus (MIDV), Bebaru virus (BEBV), O'nyong nyong virus (ONNV), Ndumu (NDUV), and Barmah Forest virus (BFV).

In one embodiment, the portion derived from the Old World alphavirus nsP3 hypervariable domain comprises a motif selected from the group consisting of: FGDF and FGSF. The portion derived from the Old World alphavirus nsP3 hypervariable domain can have a repeat selected from the group consisting of: an FGDF/FGDF repeat, an FGSF/FGSF repeat, an FGDF/FGSF repeat, and an FGSF/FGDF repeat; and the repeat sequences can be separated by at least 10 and not more than 25 amino acids. In some embodiments the repeat sequences are separated by an amino acid sequence derived from the group consisting of: SEQ ID NO: 56: NEGEIESLSSELLT, SEQ ID NO: 57: SDGEIDELSRRVTTESEPVL and SEQ ID NO: 58: DEHEVDALASGIT.

In any of the embodiments of the RNA replicons, the portion derived from the Old World alphavirus hypervariable domain can have any of amino acids 479-482 or 497-500 or 479-500 or 335-517 of CHIKV nsP3 HVD; or any of amino acids 451-454 or 468-471 or 451-471 of SFV nsP3 HVD; or amino acids 490-493 or 513-516 or 490-516 or 335-538 of SINV nsP3 HVD. In any of these embodiments (or in any embodiment described herein) the New World alphavirus can be VEEV and the portion derived from the New World alphavirus hypervariable domain does not comprise amino acids 478-518 of the VEEV nsP3 hypervariable domain; or does not comprise amino acids 478-545 of the VEEV nsP3 hypervariable domain; or does not comprise amino acids 335-518 of the VEEV nsP3 hypervariable domain. In other embodiments the New World alphavirus can be EEEV and the portion derived from the New World alphavirus hypervariable domain does not comprise amino acids 531-547 of the EEEV hypervariable domain. Or the New World alphavirus can be WEEV, and the portion derived from the New World alphavirus hypervariable domain does not comprise amino acids 504-520 of the WEEV hypervariable domain.

In some specific embodiments of the replicons, the New World alphavirus is VEEV, and the portion derived from a New World alphavirus nsP3 hypervariable domain does not comprise amino acids 335-518 of the VEEV nsP3 hypervariable domain, and the portion derived from an Old World alphavirus nsP3 hypervariable domain comprises amino acids 490-516 of SINV nsP3 HVD; or the Old World alphavirus is SINV and the portion derived from an Old World alphavirus nsP3 hypervariable domain comprises amino acids 335-538 of SINV nsP3 HVD.

In certain embodiments, an RNA replicon useful for the invention comprises RNA sub-sequences encoding amino acid sequences derived from New World alphavirus nonstructural proteins nsP1, nsP2, and nsP4; and an RNA sub-sequence encoding an amino acid sequence derived from an Old World alphavirus nsP3 protein, and wherein the first 1-6 amino acids on the N-terminal and/or C-terminal side of the nsP3 protein are derived from an New World alphavirus sequence. Thus, the 1-6 amino acids can be present on the junction between nsP2 and nsP3; or the 1-6 amino acids can be present on the junction between nsP3 and nsP4. In various embodiments the Old World alphavirus can be any described herein. When the New World alphavirus is VEEV the nsP2/nsP3 sequence can be (SEQ ID NO: 62) LHEAGC/APSY; when the junction is the nsP3/nsP4 junction the sequence can be (SEQ ID NO: 63) RFDAGA/YIFS. In any of the embodiments the penultimate glycine (also referred to by its single-letter code “G”) can be preserved and the remaining nsP3 amino acids varied as described herein. The junction sequences can optionally be preceded by a stop codon (TGA), which can be a readthrough stop codon. In other embodiments where the New World alphavirus is EEEV, the nsP2/nsP3 sequence can be (SEQ ID NO: 64) QHEAGR/APAY, and with the penultimate G preserved. When the New World alphavirus is EEEV the sequence at the nsP3/nsP4 junction can be (SEQ ID NO: 65) RYEAGA/YIFS, and the penultimate glycine can be optionally preserved while the remaining nsP3 amino acids varied as described herein. These sequences can also be preceded by a read-through stop codon (TGA). In other embodiments the New World alphavirus is WEEV, and the nsP2/nsP3 junction can be (SEQ ID NO: 66) RYEAGR/APAY, and the penultimate G preserved while the remaining amino acids in the nsP2/nsP3 junction are varied as described herein. For the nsP3/nsP4 junction of WEEV, the sequence can be (SEQ ID NO: 67) RYEAGA/YIFS, with the penultimate glycine preserved and the remaining nsP3 amino acids varied as described herein; these sequences can also be preceded by a read-through stop codon (TGA). In various embodiments the sequences (SEQ ID Nos: 62-67) can also contain one or two or three substitutions on the N-terminal and/or C-terminal sides.

Also disclosed in some embodiments include a method for producing a polypeptide of interest in a cell, which includes introducing a nucleic acid molecule according to the present disclosure into a cell, thereby producing a polypeptide encoded by the GOI in the cell. In yet another related aspect, some embodiments disclosed herein related to a method for producing a polypeptide of interest in a cell, which includes introducing an RNA molecule into the cell, wherein the RNA molecule comprises one or more RNA stem-loops of a viral capsid enhancer or a variant thereof, and a coding sequence for the polypeptide of interest, thereby producing the polypeptide of interest in the cell.

In another general aspect, the application relates to a composition comprising a self-replicating RNA molecule of the application and a pharmaceutically acceptable carrier.

In certain embodiments, the composition comprises a first polynucleotide encoding a truncated HBV core antigen, a second polynucleotide sequence encoding the HBV polymerase antigen, and a pharmaceutically acceptable carrier, wherein the first and second polynucleotides are not comprised in the same self-replicating RNA molecule. In another embodiment, the first and second polynucleotides are comprised in the same self-replicating RNA molecule.

In an embodiment, the self-replicating RNA molecule is encapsulated in, bound to or adsorbed on a liposome, a lipoplex, a lipid nanoparticle, or combinations thereof. Preferably, the self-replicating RNA molecule is encapsulated in a lipid nanoparticle.

The application further relates to a kit of the application for use in treating an HBV-induced disease in a subject in need thereof; and use of a kit of the application in the manufacture of a medicament for treating an HBV-induced disease in a subject in need thereof. The use can further comprise a combination with another therapeutic agent, preferably another anti-HBV antigen. Preferably, the subject has chronic HBV infection, and the HBV-induced disease is selected from the group consisting of advanced fibrosis, cirrhosis, and hepatocellular carcinoma (HCC).

The application also relates to a method of inducing an immune response against HBV or a method of treating an HBV infection or an HBV-induced disease, comprising administering to a subject in need thereof a self-replicating RNA or composition according to embodiments of the invention. The application further relates to a self-replicating RNA molecule of the application or a composition of the application for use in treating an HBV infection or an HBV-induced disease in a subject in need thereof.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1A and FIG. 1B show schematic representations of DNA plasmids according to embodiments of the application; FIG. 1A shows a DNA plasmid encoding an HBV core antigen according to an embodiment of the application; FIG. 1B shows a DNA plasmid encoding an HBV polymerase (pol) antigen according to an embodiment of the application; the HBV core and pol antigens are expressed under control of a CMV promoter with an N-terminal cystatin S signal peptide that is cleaved from the expressed antigen upon secretion from the cell; transcriptional regulatory elements of the plasmid include an enhancer sequence located between the CMV promoter and the polynucleotide sequence encoding the HBV antigen and a bGH polyadenylation sequence located downstream of the polynucleotide sequence encoding the HBV antigen; a second expression cassette is included in the plasmid in reverse orientation including a kanamycin resistance gene under control of an Ampr (bla) promoter; an origin of replication (pUC) is also included in reverse orientation.

FIG. 2A and FIG. 2B show the schematic representations of the expression cassettes in adenoviral vectors according to embodiments of the application; FIG. 2A shows the expression cassette for a truncated HBV core antigen, which contains a CMV promoter, an intron (a fragment derived from the human ApoAI gene-GenBank accession X01038 base pairs 295-523, harboring the ApoAI second intron), a human immunoglobulin secretion signal, followed by a coding sequence for a truncated HBV core antigen and a SV40 polyadenylation signal; FIG. 2B shows the expression cassette for a fusion protein of a truncated HBV core antigen operably linked to an HBV polymerase antigen, which is otherwise identical to the expression cassette for the truncated HBV core antigen except the HBV antigen.

FIG. 3 shows ELISPOT responses of Balb/c mice immunized with different DNA plasmids expressing HBV core antigen or HBV pol antigen, as described in Example 3; peptide pools used to stimulate splenocytes isolated from the various vaccinated animal groups are indicated in gray scale; the number of responsive T-cells are indicated on the y-axis expressed as spot forming cells (SFC) per 10⁶ splenocytes.

FIG. 4A shows the schematic representation of the alphavirus genome of the Semliki Forest virus, a positive-sensed, single-stranded RNA that encodes the non-structural polyproteins (nsP1-nsP4; replicase) at the 5′ end and structural genes (capsid and glycoproteins) at the 3′ end; and FIG. 4B shows the schematic representation of an exemplary self-amplifying RNA (saRNA) derived from alphavirus replicons, where viral structural genes are replaced by heterologous gene of interest under the transcriptional control of a subgenomic promoter (SGP). Conserved sequence elements (CSE) at the 5′ and 3′ end act as promoters for minus-strand and positive-strand RNA transcription. After the saRNA is delivered into a cell, the non-structural polyprotein precursor (nsP1234) is translated from in vitro transcribed saRNA. nsP1234 is at early stages auto-proteolytically processed to the fragments nsP123 and nsP4, which transcribes negative-stranded copies of the saRNA. Later, nsP123 is completely processed to single proteins, which assemble to the (+)strand replicase to transcribe new positive-stranded genomic copies, as well as (+)stranded subgenomic transcripts that code for the gene of interest. Subgenomic RNA as well as new genomic RNA is capped and poly-adenylated. Inactive promoters are dotted arrows; active promoters are lined arrows (Beissert et al., Hum Gene Ther. 2017, 28(12): 1138-1146).

FIG. 5A is a schematic illustration of a self-amplifying RNA derived from an alphavirus that contains a 5′cap, nonstructural genes (NSP1-4), 26S subgenomic promoter (grey arrow), the gene of interest (GOI), and a 3′ polyadenylated tail; and FIG. 5B is a schematic illustration of a lipid nanoparticle (LNP) encapsulating self-amplifying RNA, with the percent molar ratios of lipid components as indicated (Geall et al., PNAS, 2012, 109:14604-14609).

FIG. 6 is a graphical illustration of a non-limiting exemplary stem-loop RNA structure of an alphavirus capsid enhancer (e.g. DLP motif).

FIG. 7 provides a portion of the domain structure and sequence alignment of nsP3 proteins of representative members of the New World and Old World alphaviruses. The schematic representation of the nsP3 protein shows the three predicted structural domains: the macro domain, the alpha domain, and the HVD. The sequence alignment of nsP3 proteins of different alphaviruses was performed with Clustal Omega. The domain sequences are underlined with the same colors as those used in the schematic presentation. Sequences were derived from the following viruses: VEEV (GenBank accession no. P27282.2), SINV (GenBank accession no. P03317.1), SFV (GenBank accession no. NP_740667.1), CHIKV (GenBank accession no. NP_690588.1), and EEEV (GenBank accession no. Q4QXJ8.2). Image taken from Foy et al., Journal of Virology, Vol. 87, No. 4, pp. 1997-2010 (2013).

FIG. 8A is a graphical illustration of a VEEV-based alphavirus replicon encoding red firefly luciferase (rFF). Three embodiments are depicted: one having a wild-type VEEV HVD of nsP3, one a VEEV/SINV hybrid having a portion of the HVD of SINV (by substituting amino acid residues 335-538 of VEEV with amino acids 335-538 of SINV HVD; and another hybrid having a portion of CHIKV HVD (by substituting amino acid residues 335-518 of VEEV HVD with amino acids 335-517 of CHIKV HVD. FIG. 8B is a graphical illustration showing that replicons containing mutant nsP3 proteins replicate to the same levels as replicons containing wild type nsP3 and FIG. 8C is a graphical illustration showing that replicons containing mutant nsP3 proteins express the same levels of rFF as replicons containing wild type nsP3.

FIG. 9A is a graph showing the results of monitoring in vivo luciferase activity and reported as total flux. 10 ug of replicon RNA in saline was delivered intra-muscularly into the quadricep muscle of BALB/c mice. FIG. 9B shows the results of monitoring the same but with 1 ug of replicon RNA. Replicons expressing mutant forms of nsP3 exhibited similar levels of luciferase activity as replicons with wild type nsP3.

FIG. 10 is a plot and bar graph showing the results of in vivo studies of VEEV-based replicons expressing HA from H5N1 influenza virus. The data show that replicons encoding a VEEV/CHIKV HVD chimera did not elicit HA specific IgG titers compared to a replicon expressing wild type HVD.

FIG. 11A provides a plot in graphical format demonstrating the frequency of HA specific short-lived effector CD8+ T cells (SLECs) in BALB/c mice immunized with the indicated replicons expressing H5N1 HA. FIG. 11B provides a plot in graphical format demonstrating the frequency of HA specific memory precursor effector CD8+ T cells (MPECs) in BALB/c mice immunized with the indicated replicons expressing H5N1 HA.

FIG. 12 provides an illustration showing different areas of the encoded P1234 proteins of various New World and Old World viruses. G3BP binding sites are present for the following Old World virus P1234 proteins: for MAYV, amino acids 470-473; For RRV, amino acids 512-515 and 523-526; for SFV, amino acids 451-454 and 468-471; for CHIKV, amino acids 479-482 and 497-500; for ONNV, amino acids 519-522 and 537-540; for BFV, amino acids 429-432 and 447-450; for SINV, amino acids 490-493 and 513-516. For New World P1234 viral protein G3BP (and FXR) binding sites are present as follows: for VEEV, amino acids 478-545 have an FXR binding site; for EEEV, amino acids 471-483 have a G3BP binding site and amino acids 531-547 encode an FXR binding site; for WEEV, amino acids 504-520 have an FXR binding site.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL. As used herein, the use of a numerical range expressly includes all possible subranges, all individual numerical values within that range, including integers within such ranges and fractions of the values unless the context clearly indicates otherwise.

The phrases “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 91%, 92%, 93%, 94%, 95%, 97%, 98%, 99%, or 100% identity over the full-length of the amino acid sequences) can be determined, when the sequences are compared and aligned for maximum correspondence as measured using a sequence comparison algorithm as known in the art, or when manually aligned and visually inspected. The sequences which are compared to determine sequence identity can thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g. using the NCBI BLAST algorithm (Altschul S F, et al (1997), Nucleic Acids Res. 25:3389-3402).

As used herein, the terms and phrases “in combination,” “in combination with,” “co-delivery,” and “administered together with” in the context of the administration of two or more therapies or components to a subject refers to simultaneous administration or subsequent administration of two or more therapies or components, such as two vectors, e.g., RNA replicons, peptides, or a therapeutic combination and an adjuvant. “Simultaneous administration” can be administration of the two or more therapies or components at least within the same day. When two components are “administered together with” or “administered in combination with,” they can be administered in separate compositions sequentially within a short time period, such as 24, 20, 16, 12, 8 or 4 hours, or within 1 hour, or they can be administered in a single composition at the same time. “Subsequent administration” can be administration of the two or more therapies or components in the same day or on separate days. The use of the term “in combination with” does not restrict the order in which therapies or components are administered to a subject. For example, a first therapy or component (e.g. first RNA replicon encoding an HBV antigen) can be administered prior to (e.g., 5 minutes to one hour before), concomitantly with or simultaneously with, or subsequent to (e.g., 5 minutes to one hour after) the administration of a second therapy or component (e.g., second RNA replicon encoding an HBV antigen). In some embodiments, a first therapy or component (e.g. first RNA replicon encoding an HBV antigen) and a second therapy or component (e.g., second RNA replicon encoding an HBV antigen) are administered in the same composition. In other embodiments, a first therapy or component (e.g. first RNA replicon encoding an HBV antigen) and a second therapy or component (e.g., second RNA replicon encoding an HBV antigen) are administered in separate compositions, such as two separate compositions.

As used herein, a “non-naturally occurring” nucleic acid or polypeptide refers to a nucleic acid or polypeptide that does not occur in nature. A “non-naturally occurring” nucleic acid or polypeptide can be synthesized, treated, fabricated, and/or otherwise manipulated in a laboratory and/or manufacturing setting. In some cases, a non-naturally occurring nucleic acid or polypeptide can comprise a naturally-occurring nucleic acid or polypeptide that is treated, processed, or manipulated to exhibit properties that were not present in the naturally-occurring nucleic acid or polypeptide, prior to treatment. As used herein, a “non-naturally occurring” nucleic acid or polypeptide can be a nucleic acid or polypeptide isolated or separated from the natural source in which it was discovered, and it lacks covalent bonds to sequences with which it was associated in the natural source. A “non-naturally occurring” nucleic acid or polypeptide can be made recombinantly or via other methods, such as chemical synthesis.

As used herein, “subject” means any animal, preferably a mammal, most preferably a human, to whom will be or has been treated by a method according to an embodiment of the application. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

As used herein, the term “operably linked” refers to a linkage or a juxtaposition wherein the components so described are in a relationship permitting them to function in their intended manner. For example, a regulatory sequence operably linked to a nucleic acid sequence of interest is capable of directing the transcription of the nucleic acid sequence of interest, or a signal sequence operably linked to an amino acid sequence of interest is capable of secreting or translocating the amino acid sequence of interest over a membrane.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. For example, while embodiments of HBV vectors of the application (e.g., RNA replicons or viral vectors) described herein can contain particular components, including, but not limited to, certain promoter sequences, enhancer or regulatory sequences, signal peptides, coding sequence of an HBV antigen, polyadenylation signal sequences, etc. arranged in a particular order, those having ordinary skill in the art will appreciate that the concepts disclosed herein can equally apply to other components arranged in other orders that can be used in HBV vectors of the application. The application contemplates use of any of the applicable components in any combination having any sequence that can be used in HBV vectors of the application, whether or not a particular combination is expressly described. The invention generally relates to a self-replicating RNA molecule encoding one or more HBV antigens.

### Hepatitis B Virus (HBV)

As used herein “hepatitis B virus” or “HBV” refers to a virus of the hepadnaviridae family. HBV is a small (e.g., 3.2 kb) hepatotropic DNA virus that encodes four open reading frames and seven proteins. The seven proteins encoded by HBV include small (S), medium (M), and large (L) surface antigen (HBsAg) or envelope (Env) proteins, pre-Core protein, core protein, viral polymerase (Pol), and HBx protein. HBV expresses three surface antigens, or envelope proteins, L, M, and S, with S being the smallest and L being the largest. The extra domains in the M and L proteins are named Pre-S2 and Pre-S1, respectively. Core protein is the subunit of the viral nucleocapsid. Pol is needed for synthesis of viral DNA (reverse transcriptase, RNaseH, and primer), which takes place in nucleocapsids localized to the cytoplasm of infected hepatocytes. PreCore is the core protein with an N-terminal signal peptide and is proteolytically processed at its N and C termini before secretion from infected cells, as the so-called hepatitis B e-antigen (HBeAg). HBx protein is required for efficient transcription of covalently closed circular DNA (cccDNA). HBx is not a viral structural protein. All viral proteins of HBV have their own mRNA except for core and polymerase, which share an mRNA. With the exception of the protein pre-Core, none of the HBV viral proteins are subject to post-translational proteolytic processing.

The HBV virion contains a viral envelope, nucleocapsid, and single copy of the partially double-stranded DNA genome. The nucleocapsid comprises 120 dimers of core protein and is covered by a capsid membrane embedded with the S, M, and L viral envelope or surface antigen proteins. After entry into the cell, the virus is uncoated and the capsid-containing relaxed circular DNA (rcDNA) with covalently bound viral polymerase migrates to the nucleus. During that process, phosphorylation of the core protein induces structural changes, exposing a nuclear localization signal enabling interaction of the capsid with so-called importins. These importins mediate binding of the core protein to nuclear pore complexes upon which the capsid disassembles and polymerase/rcDNA complex is released into the nucleus. Within the nucleus the rcDNA becomes deproteinized (removal of polymerase) and is converted by host DNA repair machinery to a covalently closed circular DNA (cccDNA) genome from which overlapping transcripts encode for HBeAg, HBsAg, Core protein, viral polymerase and HBx protein. Core protein, viral polymerase, and pre-genomic RNA (pgRNA) associate in the cytoplasm and self-assemble into immature pgRNA-containing capsid particles, which further convert into mature rcDNA-capsids and function as a common intermediate that is either enveloped and secreted as infectious virus particles or transported back to the nucleus to replenish and maintain a stable cccDNA pool.

To date, HBV is divided into four serotypes (adr, adw, ayr, ayw) based on antigenic epitopes present on the envelope proteins, and into eight genotypes (A, B, C, D, E, F, G, and H) based on the sequence of the viral genome. The HBV genotypes are distributed over different geographic regions. For example, the most prevalent genotypes in Asia are genotypes B and C. Genotype D is dominant in Africa, the Middle East, and India, whereas genotype A is widespread in Northern Europe, sub-Saharan Africa, and West Africa.

### HBV Antigens

As used herein, the terms “HBV antigen,” “antigenic polypeptide of HBV,” “HBV antigenic polypeptide,” “HBV antigenic protein,” “HBV immunogenic polypeptide,” and “HBV immunogen” all refer to a polypeptide capable of inducing an immune response, e.g., a humoral and/or cellular mediated response, against an HBV in a subject. The HBV antigen can be a polypeptide of HBV, a fragment or epitope thereof, or a combination of multiple HBV polypeptides, portions or derivatives thereof. An HBV antigen is capable of raising in a host a protective immune response, e.g., inducing an immune response against a viral disease or infection, and/or producing an immunity (i.e., vaccinates) in a subject against a viral disease or infection, that protects the subject against the viral disease or infection. For example, an HBV antigen can comprise a polypeptide or immunogenic fragment(s) thereof from any HBV protein, such as HBeAg, pre-core protein, HBsAg (S, M, or L proteins), core protein, viral polymerase, or HBx protein derived from any HBV genotype, e.g., genotype A, B, C, D, E, F, G, and/or H, or combination thereof.

(1) HBV Core Antigen As used herein, each of the terms “HBV core antigen,” “HBc” and “core antigen” refers to an HBV antigen capable of inducing an immune response, e.g., a humoral and/or cellular mediated response, against an HBV core protein in a subject. Each of the terms “core,” “core polypeptide,” and “core protein” refers to the HBV viral core protein. Full-length core antigen is typically 183 amino acids in length and includes an assembly domain (amino acids 1 to 149) and a nucleic acid binding domain (amino acids 150 to 183). The 34-residue nucleic acid binding domain is required for pre-genomic RNA encapsidation. This domain also functions as a nuclear import signal. It comprises 17 arginine residues and is highly basic, consistent with its function. HBV core protein is dimeric in solution, with the dimers self-assembling into icosahedral capsids. Each dimer of core protein has four α-helix bundles flanked by an a-helix domain on either side. Truncated HBV core proteins lacking the nucleic acid binding domain are also capable of forming capsids.

In an embodiment of the application, an HBV antigen is a truncated HBV core antigen. As used herein, a “truncated HBV core antigen,” refers to an HBV antigen that does not contain the entire length of an HBV core protein, but is capable of inducing an immune response against the HBV core protein in a subject. For example, an HBV core antigen can be modified to delete one or more amino acids of the highly positively charged (arginine rich) C-terminal nucleic acid binding domain of the core antigen, which typically contains seventeen arginine (R) residues. A truncated HBV core antigen of the application is preferably a C-terminally truncated HBV core protein which does not comprise the HBV core nuclear import signal and/or a truncated HBV core protein from which the C-terminal HBV core nuclear import signal has been deleted. In an embodiment, a truncated HBV core antigen comprises a deletion in the C-terminal nucleic acid binding domain, such as a deletion of 1 to 34 amino acid residues of the C-terminal nucleic acid binding domain, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acid residues, preferably a deletion of all 34 amino acid residues. In a preferred embodiment, a truncated HBV core antigen comprises a deletion in the C-terminal nucleic acid binding domain, preferably a deletion of all 34 amino acid residues.

An HBV core antigen of the application can be a consensus sequence derived from multiple HBV genotypes (e.g., genotypes A, B, C, D, E, F, G, and H). As used herein, “consensus sequence” means an artificial sequence of amino acids based on an alignment of amino acid sequences of homologous proteins, e.g., as determined by an alignment (e.g., using Clustal Omega) of amino acid sequences of homologous proteins. It can be the calculated order of most frequent amino acid residues, found at each position in a sequence alignment, based upon sequences of HBV antigens (e.g., core, pol, etc.) from at least 100 natural HBV isolates. A consensus sequence can be non-naturally occurring and different from the native viral sequences. Consensus sequences can be designed by aligning multiple HBV antigen sequences from different sources using a multiple sequence alignment tool, and at variable alignment positions, selecting the most frequent amino acid. Preferably, a consensus sequence of an HBV antigen is derived from HBV genotypes B, C, and D. The term “consensus antigen” is used to refer to an antigen having a consensus sequence.

An exemplary truncated HBV core antigen according to the application lacks the nucleic acid binding function, and is capable of inducing an immune response in a mammal against at least two HBV genotypes. Preferably a truncated HBV core antigen is capable of inducing a T cell response in a mammal against at least HBV genotypes B, C and D. More preferably, a truncated HBV core antigen is capable of inducing a CD8+ T cell response in a human subject against at least HBV genotypes A, B, C and D.

Preferably, an HBV core antigen of the application is a consensus antigen, preferably a consensus antigen derived from HBV genotypes B, C, and D, more preferably a truncated consensus antigen derived from HBV genotypes B, C, and D. An exemplary truncated HBV core consensus antigen according to the application consists of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100% identical to SEQ ID NO: 2 or SEQ ID NO: 4. SEQ ID NO: 2 and SEQ ID NO: 4 are core consensus antigens derived from HBV genotypes B, C, and D. SEQ ID NO: 2 and SEQ ID NO: 4 each contain a 34-amino acid C-terminal deletion of the highly positively charged (arginine rich) nucleic acid binding domain of the native core antigen.

In one embodiment of the application, an HBV core antigen is a truncated HBV antigen consisting of the amino acid sequence of SEQ ID NO: 2. In another embodiment, an HBV core antigen is a truncated HBV antigen consisting of the amino acid sequence of SEQ ID NO: 4. In another embodiment, an HBV core antigen further contains a signal sequence operably linked to the N-terminus of a mature HBV core antigen sequence, such as the amino acid sequence of SEQ ID NO: 2 or SEQ ID NO: 4. Preferably, the signal sequence has the amino acid sequence of SEQ ID NO: 9 or SEQ ID NO: 15.

### (2) HBV Polymerase Antigen

As used herein, the term “HBV polymerase antigen,” “HBV Pol antigen” or “HBV pol antigen” refers to an HBV antigen capable of inducing an immune response, e.g., a humoral and/or cellular mediated response, against an HBV polymerase in a subject. Each of the terms “polymerase,” “polymerase polypeptide,” “Pol” and “pol” refers to the HBV viral DNA polymerase. The HBV viral DNA polymerase has four domains, including, from the N terminus to the C terminus, a terminal protein (TP) domain, which acts as a primer for minus-strand DNA synthesis; a spacer that is nonessential for the polymerase functions; a reverse transcriptase (RT) domain for transcription; and an RNase H domain.

In an embodiment of the application, an HBV antigen comprises an HBV Pol antigen, or any immunogenic fragment or combination thereof. An HBV Pol antigen can contain further modifications to improve immunogenicity of the antigen, such as by introducing mutations into the active sites of the polymerase and/or RNase domains to decrease or substantially eliminate certain enzymatic activities.

Preferably, an HBV Pol antigen of the application does not have reverse transcriptase activity and RNase H activity and is capable of inducing an immune response in a mammal against at least two HBV genotypes. Preferably, an HBV Pol antigen is capable of inducing a T cell response in a mammal against at least HBV genotypes B, C and D. More preferably, an HBV Pol antigen is capable of inducing a CD8+ T cell response in a human subject against at least HBV genotypes A, B, C and D.

Thus, in some embodiments, an HBV Pol antigen is an inactivated Pol antigen. In an embodiment, an inactivated HBV Pol antigen comprises one or more amino acid mutations in the active site of the polymerase domain. In another embodiment, an inactivated HBV Pol antigen comprises one or more amino acid mutations in the active site of the RNaseH domain. In a preferred embodiment, an inactivated HBV pol antigen comprises one or more amino acid mutations in the active site of both the polymerase domain and the RNaseH domain. For example, the “YXDD” motif in the polymerase domain of an HBV pol antigen that can be required for nucleotide/metal ion binding can be mutated, e.g., by replacing one or more of the aspartate residues (D) with asparagine residues (N), eliminating or reducing metal coordination function, thereby decreasing or substantially eliminating reverse transcriptase function. Alternatively, or in addition to mutation of the “YXDD” motif, the “DEDD” motif in the RNaseH domain of an HBV pol antigen required for Mg2+ coordination can be mutated, e.g., by replacing one or more aspartate residues (D) with asparagine residues (N) and/or replacing the glutamate residue (E) with glutamine (Q), thereby decreasing or substantially eliminating RNaseH function. In a particular embodiment, an HBV pol antigen is modified by (1) mutating the aspartate residues (D) to asparagine residues (N) in the “YXDD” motif of the polymerase domain; and (2) mutating the first aspartate residue (D) to an asparagine residue (N) and the first glutamate residue (E) to a glutamine residue (N) in the “DEDD” motif of the RNaseH domain, thereby decreasing or substantially eliminating both the reverse transcriptase and RNaseH functions of the pol antigen.

In a preferred embodiment of the application, an HBV pol antigen is a consensus antigen, preferably a consensus antigen derived from HBV genotypes B, C, and D, more preferably an inactivated consensus antigen derived from HBV genotypes B, C, and D. An exemplary HBV pol consensus antigen according to the application comprises an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably at least 98% identical to SEQ ID NO: 7, such as at least 98%, 98.5%, 99%, 99.1%, 99.2%, 99.3%, 99.4%, 99.5%, 99.6%, 99.7%, 99.8%, 99.9% or 100% identical to SEQ ID NO: 7. SEQ ID NO: 7 is a pol consensus antigen derived from HBV genotypes B, C, and D comprising four mutations located in the active sites of the polymerase and RNaseH domains. In particular, the four mutations include mutation of the aspartic acid residues (D) to asparagine residues (N) in the “YXDD” motif of the polymerase domain; and mutation of the first aspartate residue (D) to an asparagine residue (N) and mutation of the glutamate residue (E) to a glutamine residue (Q) in the “DEDD” motif of the RNaseH domain.

In a particular embodiment of the application, an HBV pol antigen comprises the amino acid sequence of SEQ ID NO: 7. In other embodiments of the application, an HBV pol antigen consists of the amino acid sequence of SEQ ID NO: 7. In a further embodiment, an HBV pol antigen further contains a signal sequence operably linked to the N-terminus of a mature HBV pol antigen sequence, such as the amino acid sequence of SEQ ID NO: 7. Preferably, the signal sequence has the amino acid sequence of SEQ ID NO: 9 or SEQ ID NO: 15.

### (3) Fusion of HBV Core Antigen and HBV Polymerase Antigen

As used herein the term “fusion protein” or “fusion” refers to a single polypeptide chain having at least two polypeptide domains that are not normally present in a single, natural polypeptide.

In an embodiment of the application, an HBV antigen comprises a fusion protein comprising a truncated HBV core antigen operably linked to an HBV Pol antigen, or an HBV Pol antigen operably linked to a truncated HBV core antigen, preferably via a linker. For example, in a fusion protein containing a first polypeptide and a second heterologous polypeptide, a linker serves primarily as a spacer between the first and second polypeptides. In an embodiment, a linker is made up of amino acids linked together by peptide bonds, preferably from 1 to 20 amino acids linked by peptide bonds, wherein the amino acids are selected from the 20 naturally occurring amino acids. In an embodiment, the 1 to 20 amino acids are selected from glycine, alanine, proline, asparagine, glutamine, and lysine. Preferably, a linker is made up of a majority of amino acids that are sterically unhindered, such as glycine and alanine. Exemplary linkers are polyglycines, particularly (Gly)5, (Gly)8; poly(Gly-Ala), and polyalanines. One exemplary suitable linker as shown in the Examples below is (AlaGly)n, wherein n is an integer of 2 to 5.

Preferably, a fusion protein of the application is capable of inducing an immune response in a mammal against HBV core and HBV Pol of at least two HBV genotypes. Preferably, a fusion protein is capable of inducing a T cell response in a mammal against at least HBV genotypes B, C and D. More preferably, the fusion protein is capable of inducing a CD8+ T cell response in a human subject against at least HBV genotypes A, B, C and D.

In an embodiment of the application, a fusion protein comprises a truncated HBV core antigen having an amino acid sequence at least 90%, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100% identical to SEQ ID NO: 2 or SEQ ID NO: 4, a linker, and an HBV Pol antigen having an amino acid sequence at least 90%, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100%, identical to SEQ ID NO: 7.

In a preferred embodiment of the application, a fusion protein comprises a truncated HBV core antigen consisting of the amino acid sequence of SEQ ID NO: 2 or SEQ ID NO: 4, a linker comprising (AlaGly)n, wherein n is an integer of 2 to 5, and an HBV Pol antigen having the amino acid sequence of SEQ ID NO: 7. More preferably, a fusion protein according to an embodiment of the application comprises the amino acid sequence of SEQ ID NO: 16.

In one embodiment of the application, a fusion protein further comprises a signal sequence operably linked to the N-terminus of the fusion protein. Preferably, the signal sequence has the amino acid sequence of SEQ ID NO: 9 or SEQ ID NO: 15. In one embodiment, a fusion protein comprises the amino acid sequence of SEQ ID NO: 17.

Additional disclosure on HBV vaccines that can be used for the present invention are described in U.S. patent application Ser. No: 16/223,251, filed Dec. 18, 2018, the contents of the application, more preferably the examples, are hereby incorporated by reference in their entireties.

Polynucleotides and Vectors In another general aspect, the application provides a non-naturally occurring nucleic acid molecule encoding an HBV antigen useful for an invention according to embodiments of the application, and vectors comprising the non-naturally occurring nucleic acid. A first or second non-naturally occurring nucleic acid molecule can comprise any polynucleotide sequence encoding an HBV antigen useful for the application, which can be made using methods known in the art in view of the present disclosure. Preferably, a first or second polynucleotide encodes at least one of a truncated HBV core antigen and an HBV polymerase antigen of the application. A polynucleotide can be in the form of RNA or in the form of DNA obtained by recombinant techniques (e.g., cloning) or produced synthetically (e.g., chemical synthesis). The DNA can be single-stranded or double-stranded, or can contain portions of both double-stranded and single-stranded sequence. The DNA can, for example, comprise genomic DNA, cDNA, or combinations thereof. The polynucleotide can also be a DNA/RNA hybrid. The polynucleotides and vectors of the application can be used for recombinant protein production, expression of the protein in host cell, or the production of viral particles. Preferably, a polynucleotide is RNA.

In an embodiment of the application, a first non-naturally occurring nucleic acid molecule comprises a first polynucleotide sequence encoding a truncated HBV core antigen consisting of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2, preferably 98%, 99% or 100% identical to SEQ ID NO: 2 or SEQ ID NO: 4. In a particular embodiment of the application, a first non-naturally occurring nucleic acid molecule comprises a first polynucleotide sequence encoding a truncated HBV core antigen consisting the amino acid sequence of SEQ ID NO: 2 or SEQ ID NO: 4.

Examples of polynucleotide sequences of the application encoding a truncated HBV core antigen consisting of the amino acid sequence of SEQ ID NO: 2 or SEQ ID NO: 4 include, but are not limited to, a polynucleotide sequence at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3. Exemplary non-naturally occurring nucleic acid molecules encoding a truncated HBV core antigen have the polynucleotide sequence of SEQ ID NOs: 1 or 3.

In another embodiment, a first non-naturally occurring nucleic acid molecule further comprises a coding sequence for a signal sequence that is operably linked to the N-terminus of the HBV core antigen sequence. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14.

In an embodiment of the application, a second non-naturally occurring nucleic acid molecule comprises a second polynucleotide sequence encoding an HBV polymerase antigen comprising an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably 100% identical to SEQ ID NO: 7. In a particular embodiment of the application, a second non-naturally occurring nucleic acid molecule comprises a second polynucleotide sequence encoding an HBV polymerase antigen consisting of the amino acid sequence of SEQ ID NO: 7.

Examples of polynucleotide sequences of the application encoding an HBV Pol antigen comprising the amino acid sequence of at least 90% identical to SEQ ID NO: 7 include, but are not limited to, a polynucleotide sequence at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6. Exemplary non-naturally occurring nucleic acid molecules encoding an HBV pol antigen have the polynucleotide sequence of SEQ ID NOs: 5 or 6.

In another embodiment, a second non-naturally occurring nucleic acid molecule further comprises a coding sequence for a signal sequence that is operably linked to the N-terminus of the HBV pol antigen sequence, such as the amino acid sequence of SEQ ID NO: 7. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14.

In another embodiment of the application, a non-naturally occurring nucleic acid molecule encodes an HBV antigen fusion protein comprising a truncated HBV core antigen operably linked to an HBV Pol antigen, or an HBV Pol antigen operably linked to a truncated HBV core antigen. In a particular embodiment, a non-naturally occurring nucleic acid molecule of the application encodes a truncated HBV core antigen consisting of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2 or SEQ ID NO: 4, preferably 100% identical to SEQ ID NO: 2 or SEQ ID NO: 4, more preferably 100% identical to SEQ ID NO: 2 or SEQ ID NO:4; a linker; and an HBV polymerase antigen comprising an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably 98%, 99% or 100% identical to SEQ ID NO: 7. In a particular embodiment of the application, a non-naturally occurring nucleic acid molecule encodes a fusion protein comprising a truncated HBV core antigen consisting of the amino acid sequence of SEQ ID NO: 2 or SEQ ID NO: 4, a linker comprising (AlaGly)n, wherein n is an integer of 2 to 5; and an HBV Pol antigen comprising the amino acid sequence of SEQ ID NO: 7. In a particular embodiment of the application, a non-naturally occurring nucleic acid molecule encodes an HBV antigen fusion protein comprising the amino acid sequence of SEQ ID NO: 16.

Examples of polynucleotide sequences of the application encoding an HBV antigen fusion protein include, but are not limited to, a polynucleotide sequence at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3, operably linked to a linker coding sequence at least 90% identical to SEQ ID NO: 11, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 11, preferably 98%, 99% or 100% identical to SEQ ID NO: 11, which is further operably linked a polynucleotide sequence at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6. In particular embodiments of the application, a non-naturally occurring nucleic acid molecule encoding an HBV antigen fusion protein comprises SEQ ID NO: 1 or SEQ ID NO: 3, operably linked to SEQ ID NO: 11, which is further operably linked to SEQ ID NO: 5 or SEQ ID NO: 6.

In another embodiment, a non-naturally occurring nucleic acid molecule encoding an HBV fusion further comprises a coding sequence for a signal sequence that is operably linked to the N-terminus of the HBV fusion sequence, such as the amino acid sequence of SEQ ID NO: 16. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14. In one embodiment, the encoded fusion protein with the signal sequence comprises the amino acid sequence of SEQ ID NO: 17.

The application also relates to a vector comprising the first and/or second non-naturally occurring nucleic acid molecules. As used herein, a “vector” is a nucleic acid molecule used to carry genetic material into another cell, where it can be replicated and/or expressed. A vector of the application can be an expression vector. As used herein, the term “expression vector” refers to any type of genetic construct comprising a nucleic acid coding for an RNA capable of being transcribed. Expression vectors include, but are not limited to, vectors for recombinant protein expression, such as an RNA replicon or a viral vector, and vectors for delivery of nucleic acid into a subject for expression in a tissue of the subject, such as an RNA replicon or a viral vector. It will be appreciated by those skilled in the art that the design of the expression vector can depend on such factors as the choice of the host cell to be transformed, the level of expression of protein desired, etc.

Vectors of the application can contain a variety of regulatory sequences. As used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. In the context of the disclosure, this term encompasses promoters, enhancers and other expression control elements (e.g., polyadenylation signals and elements that affect mRNA stability).

Preferably, the vector is a self-replicating RNA replicon.

As used herein, “self-replicating RNA molecule,” which is used interchangeably with “self-amplifying RNA molecule” or “RNA replicon” or “replicon RNA” or “saRNA,” refers to an RNA molecule engineered from genomes of plus-strand RNA viruses that contains all of the genetic information required for directing its own amplification or self-replication within a permissive cell. A self-replicating RNA molecule resembles mRNA. It is single-stranded, 5′-capped, and 3′-poly-adenylated and is of positive orientation. To direct its own replication, the RNA molecule 1) encodes polymerase, replicase, or other proteins which can interact with viral or host cell-derived proteins, nucleic acids or ribonucleoproteins to catalyze the RNA amplification process; and 2) contain cis-acting RNA sequences required for replication and transcription of the subgenomic replicon-encoded RNA. Thus, the delivered RNA leads to the production of multiple daughter RNAs. These daughter RNAs, as well as collinear subgenomic transcripts, can be translated themselves to provide in situ expression of a gene of interest, or can be transcribed to provide further transcripts with the same sense as the delivered RNA which are translated to provide in situ expression of the gene of interest. The overall result of this sequence of transcriptions is a huge amplification in the number of the introduced replicon RNAs and so the encoded gene of interest becomes a major polypeptide product of the cells.

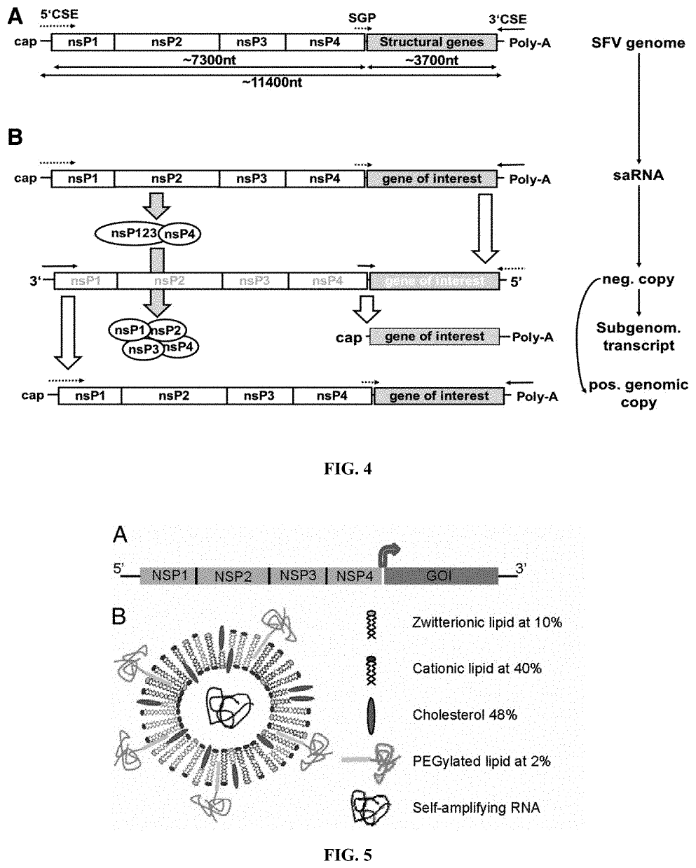

In certain embodiments, a self-replicating RNA molecule encodes an enzyme complex for self-amplification (replicase polyprotein) comprising an RNA-dependent RNA-polymerase function, helicase, capping, and poly-adenylating activity. The viral structural genes downstream of the replicase, which are under control of a subgenomic promoter, can be replaced by genes of interest (GOI). Upon transfection, the replicase is translated immediately, interacts with the 5′ and 3′ termini of the genomic RNA, and synthesizes complementary genomic RNA copies. Those act as templates for the synthesis of novel positive-stranded, capped, and poly-adenylated genomic copies, and subgenomic transcripts (FIG. 4). Amplification eventually leads to very high RNA copy numbers of up to 2×10⁵ copies per cell. Thus, much lower amounts of saRNA compared to conventional mRNA suffice to achieve effective gene transfer and protective vaccination (Beissert et al., Hum Gene Ther. 2017, 28(12): 1138-1146).

Subgenomic RNA is an RNA molecule of a length or size which is smaller than the genomic RNA from which it was derived. The viral subgenomic RNA can be transcribed from an internal promoter, whose sequences reside within the genomic RNA or its complement. Transcription of a subgenomic RNA can be mediated by viral-encoded polymerase(s) associated with host cell-encoded proteins, ribonucleoprotein(s), or a combination thereof. Numerous RNA viruses generate subgenomic mRNAs (sgRNAs) for expression of their 3′-proximal genes.

In some embodiments of the present disclosure, one or more genes of interest (e.g. HBV antigen genes) are expressed under the control of a subgenomic promoter. In certain embodiments, instead of the native subgenomic promoter, the subgenomic RNA can be placed under control of internal ribosome entry site (IRES) derived from encephalomyocarditis viruses (EMCV), Bovine Viral Diarrhea Viruses (BVDV), polioviruses, Foot-and-mouth disease viruses (FMD), enterovirus 71, or hepatitis C viruses. Subgenomic promoters range from 24 nucleotide (Sindbis virus) to over 100 nucleotides (Beet necrotic yellow vein virus) and are usually found upstream of the transcription start.

In some embodiments, the RNA replicon includes the coding sequence for at least one, at least two, at least three, or at least four nonstructural viral proteins (e.g. nsP1, nsP2, nsP3, nsP4). In some embodiments, RNA replicon includes the coding sequence for a portion of the at least one nonstructural viral protein. For example, the RNA replicon can include about 10%, 20%, 30%, 40%, 50%, 60%, 70%, 80%, 90%, 95%, 100%, or a range between any two of these values, of the encoding sequence for the at least one nonstructural viral protein. In some embodiments, the RNA replicon can include the coding sequence for a substantial portion of the at least one nonstructural viral protein. As used herein, a “substantial portion” of a nucleic acid sequence encoding a nonstructural viral protein comprises enough of the nucleic acid sequence encoding the nonstructural viral protein to afford putative identification of that protein, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993). In some embodiments, the RNA replicon can include the entire coding sequence for the at least one nonstructural protein. In some embodiments, the RNA replicon comprises substantially all the coding sequence for the native viral nonstructural proteins. In certain embodiments, the one or more nonstructural viral proteins are derived from the same virus. In other embodiments, the one or more nonstructural proteins are derived from different viruses.

The RNA replicon can be derived from any suitable plus-strand RNA viruses, such as alphaviruses or flaviviruses. Preferably, the RNA replicon is derived from alphaviruses. The term “alphavirus” describes enveloped single-stranded positive sense RNA viruses of the family Togaviridae. The genus alphavirus contains approximately 30 members, which can infect humans as well as other animals. Alphavirus particles typically have a 70 nm diameter, tend to be spherical or slightly pleomorphic, and have a 40 nm isometric nucleocapsid. The total genome length of alphaviruses ranges between 11,000 and 12,000 nucleotides and has a 5′cap and 3′ poly-A tail. There are two open reading frames (ORF's) in the genome, non-structural (ns) and structural. The ns ORF encodes proteins (nsP1-nsP4) necessary for transcription and replication of viral RNA. The structural ORF encodes three structural proteins: the core nucleocapsid protein C, and the envelope proteins P62 and E1 that associate as a heterodimer. The viral membrane-anchored surface glycoproteins are responsible for receptor recognition and entry into target cells through membrane fusion. The four ns protein genes are encoded by genes in the 5′ two-thirds of the genome, while the three structural proteins are translated from a subgenomic mRNA colinear with the 3′ one-third of the genome. An exemplary depiction of an alphavirus genome is shown in FIG. 4A.

In some embodiments, the self-replicating RNA useful for the invention is an RNA replicon derived from an alphavirus virus species. In some embodiments, the alphavirus RNA replicon is of an alphavirus belonging to the VEEV/EEEV group, or the SF group, or the SIN group. Non-limiting examples of SF group alphaviruses include Semliki Forest virus, O'Nyong-Nyong virus, Ross River virus, Middelburg virus, Chikungunya virus, Barmah Forest virus, Getah virus, Mayaro virus, Sagiyama virus, Bebaru virus, and Una virus. Non-limiting examples of SIN group alphaviruses include Sindbis virus, Girdwood S. A. virus, South African Arbovirus No. 86, Ockelbo virus, Aura virus, Babanki virus, Whataroa virus, and Kyzylagach virus. Non-limiting examples of VEEV/EEEV group alphaviruses include Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), and Una virus (UNAV).

Non-limiting examples of alphavirus species include Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), and Buggy Creek virus. Virulent and avirulent alphavirus strains are both suitable. In some embodiments, the alphavirus RNA replicon is of a Sindbis virus (SIN), a Semliki Forest virus (SFV), a Ross River virus (RRV), a Venezuelan equine encephalitis virus (VEEV), or an Eastern equine encephalitis virus (EEEV). In some embodiments, the alphavirus RNA replicon is of a Venezuelan equine encephalitis virus (VEEV).

In certain embodiments, a self-replicating RNA molecule comprises a polynucleotide encoding one or more nonstructural proteins nsP1-4, a subgenomic promoter, such as 26S subgenomic promoter, and a gene of interest encoding one or more of the HBV antigens described herein.

A self-replicating RNA molecule can have a 5′ cap (e.g. a 7-methylguanosine). This cap can enhance in vivo translation of the RNA.

The 5′ nucleotide of a self-replicating RNA molecule useful with the invention can have a 5′ triphosphate group. In a capped RNA this can be linked to a 7-methylguanosine via a 5′-to-5′ bridge. A 5′ triphosphate can enhance RIG-I binding.

A self-replicating RNA molecule can have a 3′ poly-A tail. It can also include a poly-A polymerase recognition sequence (e.g. AAUAAA) near its 3′ end.

In some embodiments, the replicon RNA does not contain coding sequences for at least one of the structural viral proteins. In these instances, the sequences encoding structural genes can be substituted with one or more heterologous sequences such as, for example, a coding sequence for a gene of interest (e.g., an HBV antigen). See, FIG. 4B.

In those instances where the replicon RNA is to be packaged into a recombinant alphavirus particle, it must contain one or more sequences, so-called packaging signals, which serve to initiate interactions with alphavirus structural proteins that lead to particle formation. In certain embodiments, the alphavirus particles comprise RNA derived from one or more alphaviruses; and structural proteins wherein at least one of said structural proteins is derived from two or more alphaviruses.

Double-stranded RNA (dsRNA) intermediates are formed during saRNA translation. The dsRNA intermediates are natural ligands of cytoplasmic RNA sensors such as Rig-I, MDAS, and protein kinase R (PKR). The interaction between the dsRNA and cytoplasmic RNA sensors results in activation of the interferon response genes and strong intrinsic adjuvant activity of saRNA. However, activation of cytoplasmic RNA sensors, particularly PKR, also results in a general inhibition of translation. Activated PKR phosphorylates the eukaryotic initiation factor 2 alpha subunit (eIF2α), thereby blocking cap-dependent translation, including that of saRNA. As a counter-mechanism to rescue translation, alphaviruses evolved an RNA stem-loop structure downstream of the capsid start codon (downstream loop, DLP) spanning the 5′-terminal 102 nucleotides (34 amino acids) of the capsid ORF, providing eIF2α independent translation. Replacing the capsid ORF with a GOI can result in a recombinant saRNA that lacks a DLP and regains sensitivity toward activated PKR, resulting in suppression of the expression of the GOI. However, a fusion of the DLP spanning part of the capsid to the GOI bears the risk of a functional alteration to the GOI (Beissert et al., Hum Gene Ther. 2017, 28(12): 1138-1146).

In certain embodiments, a self-replicating RNA vector of the application comprises one or more features to confer a resistance to the translation inhibition by the innate immune system or to otherwise increase the expression of the GOI (e.g., an HBV gene). For example, a self-replicating RNA of the application can be co-delivered with a non-replicating mRNA encoding vaccinia virus immune evasion proteins E3, K3, and B18. It was shown that E3 is superior to K3 or B18 as a highly potent blocker of PKR activation and of interferon (IFN)-β upregulation. B18, in contrast, is superior in controlling OAS1, a key IFN-inducible gene involved in viral RNA degradation. By combining all three vaccinia proteins, a significant suppression of PKR and IFN pathway activation in vitro can be achieved, resulting in enhanced expression of saRNA-encoded genes of interest both in vitro and in vivo (Beissert et al., Hum Gene Ther. 2017, 28(12): 1138-1146).

In certain embodiments, the RNA sequence can be codon optimized to improve translation efficiency. The RNA molecule can be modified by any method known in the art in view of the present disclosure to enhance stability and/or translation, such by adding a polyA tail, e.g., of at least 30 adenosine residues; and/or capping the 5-end with a modified ribonucleotide, e.g., 7-methylguanosine cap, which can be incorporated during RNA synthesis or enzymatically engineered after RNA transcription.

In certain embodiments, a self-replicating RNA vector of the application comprises a DLP motif.

As used herein, a “downstream loop” or “DLP motif” refers to a polynucleotide sequence comprising at least one RNA stem-loop, which when placed downstream of a start codon of an open reading frame (ORF) provides increased translation the ORF compared to an otherwise identical construct without the DLP motif. For example, a DLP motif can provide eIF2α independent translation. In one embodiment, DLP motif is derived from a capsid gene of a virus species belonging to the Togaviridae family. In one embodiment, the self-replicating RNA molecule also contains a coding sequence for an autoprotease peptide operably linked downstream of the DLP motif and upstream of the GOI. Examples of the autoprotease peptide include, but are not limited to, a peptide sequence selected from the group consisting of porcine teschovirus-1 2A (P2A), a foot-and-mouth disease virus (FMDV) 2A (F2A), an Equine Rhinitis A virus (ERAV) 2A (E2A), a Thosea asigna virus 2A (T2A), a cytoplasmic polyhydrosis virus 2A (BmCPV2A), a Flacherie Virus 2A (BmIFV2A), and a combination thereof. Examples of a self-replicating RNA vector comprising a DLP motif are described in US Patent Application Publication US2018/0171340 and the International Patent Application Publication WO2018106615, the content of which is incorporated herein by reference in its entirety.

In another embodiment, a self-replicating RNA replicon of the application comprises a modified 5′ untranslated region (5′-UTR), preferably the RNA replicon is devoid of at least a portion of a nucleic acid sequence encoding viral structural proteins. For example, the modified 5′-UTR can comprise one or more nucleotide substitutions at position 1, 2, 4, or a combination thereof. Preferably, the modified 5′-UTR comprises a nucleotide substitution at position 2, more preferably, the modified 5′-UTR has a U->G substitution at position 2. Examples of such self-replicating RNA molecules are described in US Patent Application Publication US2018/0104359 and the International Patent Application Publication WO2018075235, the content of which is incorporated herein by reference in its entirety.

Previous detailed analyses of the 5′-unstranslated regions (5′-UTR) of alphaviruses have revealed the absolute importance of the extreme 5′ nucleotides to support RNA replication and transcription. In particular, the conservation of an AU dinucleotide at nucleotide positions 1 and 2, respectively, of the 5′ UTR sequence is noted among all alphaviruses suggesting the importance of these nucleotides. As used herein, “AI” refers to the conserved A nucleotide at nucleotide position 1 of the 5′-UTR (e.g., an alphavirus 5′-UTR), and “U2” refers to the conserved U nucleotide at nucleotide position 2 of the 5′-UTR (e.g., an alphavirus 5′-UTR). Further, for Venezuelan equine encephalitis virus (VEEV), detailed analysis of the 5′ most three nucleotides as well as the stem loop region found immediately following this sequence has been conducted. In particular, the importance of maintaining the U residue at position 2 of the 5′ UTR has been determined previously (Kulasegaran-Shylini et al., J. Virol. 83:17 p 8327-8339, 2009a; and Kulasegaran-Shylini et al. J. Virol. 83:17 p 8327-8339, 2009b). Specifically, in vitro transcribed RNA from a full length infectious clone designated (G2)VEE/SINV containing a single U2->G change in the 5′ UTR demonstrated a loss of nearly three logs of infectivity compared to in vitro transcribed RNA from a wild type VEE/SINV infectious clone. This report strongly suggests that the U at position 2 is critical to RNA replication and cannot be replaced with a G. However, as described herein in details, a VEEV replicon with a U2->G change in the 5′ UTR is, surprisingly and in direct contradiction to this previous report, not only completely capable of robust replication but result in three times the expression potential of a VEEV replicon as compared to a wild-type 5′ UTR containing the U residue at position 2.

The extreme 5′ and 3′ sequences of most RNA viruses are highly constrained and little if any variation is tolerated; most modifications result in highly crippled or lethal outcomes for RNA replication. Kulasegaran-Shylini et al. completed an in-depth analysis of the 5′ nucleotide sequences critical to RNA replication for a chimeric VEEV/SINV infectious clone, which is representative of all alphaviruses (Kulasegaran-Shylini et al. 2009a, supra). The Kulasegaran-Shylini et al. 2009b paper (J. Virol. 83:17 p 8327-8339, 2009) specifically states/shows that changing nucleotide 2 in the 5′ UTR from a U residue to a G residue (U2->G) significantly reduces the viability of that infectious clone RNA. That is, that specific change in the 5′-UTR reduced biologic activity of the infectious clone RNA by nearly 3 orders of magnitude. As disclosed herein, the change in the 5′-UTR (e.g., a U2->G change) incorporated into a VEEV (strain TC-83) replicon RNA not only does not cripple the replication of the replicon but can actually increase the biological activity of the replicon. For example, the replicon comprising the U2->G substitution can, in some embodiments, lead to the expression of a protein of interest as much as three times more than a wild type replicon expressing the same protein. This result is surprising and the increased biologic activity of the replicon carrying the U2->G change could not have been predicted. This modified replicon has the potential to be a superior RNA expression platform to support both vaccine and therapeutic applications.

Conservation of the 5′ most 2 nucleotides has been observed across all of the genomic RNA of alphavirus subtypes. The conserved AU dinucleotide (A1 and U2) has also been shown to be critically required for RNA replication (Kulasegaran-Shylini et al. 2009a and 2009b, supra). The demonstration that an alphavirus replicon RNA carrying an AG dinucleotide at the extreme 5′ end is not only completely functional but demonstrates enhanced biologic activity is surprising and is completely contrary to the dogma in the field.

Monogenic or multigenic alphavirus expression systems can be generated by using a modified replicon RNA having expression/translation enhancing activity such as, for example, a replicon RNA containing a modified 5′-UTR. In some embodiments, the viral (e.g., alphavirus) expression systems as described herein are further devoid of a part or the entire coding region for one or more viral structural proteins. For example, the alphavirus expression system may be devoid of a portion of or the entire coding sequence for one or more of the viral capsid protein C, E1 glycoprotein, E2 glycoprotein, E3 protein and 6K protein. In some embodiments, modification of nucleotide at position 2 in a cDNA copy of the Venezuelan equine encephalitis virus (VEEV) 5′ UTR sequence from a thymine (T) nucleotide to a guanine (G) nucleotide (T2->G mutation), in the context of a replicon RNA, bestows the replicon with significantly higher protein expression potential compared to a VEEV replicon with a wild-type 5′ UTR sequence.

In some embodiments, the level of expression and/or translation enhancement activity of the modified replicon RNAs as disclosed herein is of at least 1.1, 1.2, 1.3, 1.4, 1.5, 1.6, 1.7, 1.8, 1.9, 2 (2-fold), 3, 4, 5, 6, 7, 8, or more times, relative to the expression level detected from a corresponding unmodified replicon, e.g. replicon with a wild-type 5′ UTR. Without being limited by any particular theory, enhanced translation can be due to an enhancement of transcription which results in an increased level of transcripts being available for translation and/or can be independent of transcription and be due to for example enhanced ribosome binding. The level of enhancement activity can be measured by any convenient methods and techniques known in the art including, but are not limited to, transcript level, amount of protein, protein activity, etc.

In one aspect, novel nucleic acid molecules which include a modified replicon RNA are disclosed herein. For example, a modified replicon RNA can comprise mutation(s), deletion(s), substitution(s), and/or insertion(s) in one or more of the original genomic regions (e.g., open reading frames (ORFs) and/or non-coding regions (e.g., promoter sequences)) of the parent replicon RNA. In some embodiments, the modified replicon RNA includes a modified 5′-untranslated region (5′-UTR). In some embodiments, the modified 5′-UTR includes one or more nucleotide substitutions at position 1, 2, 4, or a combination thereof. In some embodiments, at least one of the nucleotide substitutions is a nucleotide substitution at position 1 of the modified 5′-UTR. In some embodiments, at least one of the nucleotide substitutions is a nucleotide substitution at position 2 of the modified 5′-UTR. In some embodiments, at least one of the nucleotide substitutions is a nucleotide substitution at position 4 of the modified 5′-UTR. In some embodiments, the nucleotide substitution at position 2 of the modified 5′-UTR is a U->G substitution. In some embodiments, the nucleotide substitution at position 2 of the modified 5′-UTR is a U->A substitution. In some embodiments, the nucleotide substitution at position 2 of the modified 5′-UTR is a U->C substitution.

In some embodiments, the nucleic acid molecule as disclosed herein includes a modified alphavirus genome or replicon RNA, wherein the modified alphavirus genome or replicon RNA comprises a 5′-UTR exhibiting at least 80% sequence identity to the nucleic acid sequence of at least one 5′-UTR disclosed herein and a U->G substitution at position 2 of the 5′-UTR, and wherein the modified alphavirus genome or replicon RNA is devoid of at least a portion of the sequence encoding viral structural proteins. In some embodiments, the modified alphavirus genome or replicon RNA comprises a 5′-UTR exhibiting at least 80% sequence identity to at least one of the sequences set forth in SEQ ID NOs: 26-42. In some embodiments, the modified alphavirus genome or replicon RNA comprises a 5′-UTR exhibiting at least 90%, at least 95%, at least 96%, at least 97%, at least 98%, at least 99%, or 100% sequence identity to at least one of the sequences set forth in SEQ ID NOs: 26-42. In some embodiments, the modified alphavirus genome or replicon RNA comprises a 5′-UTR exhibiting 100% sequence identity to at least one of the sequences set forth in SEQ ID NOs: 26-42 of the Sequence Listing.

In various embodiments disclosed herein, the nucleic acid molecule disclosed herein can include one or more of the following features. In some embodiments, the modified replicon RNA is a modified alphavirus replicon RNA. In some embodiments, the modified alphavirus replicon RNA includes a modified alphavirus genome. In some embodiments, the modified 5′-UTR includes one or more nucleotide substitutions at position 1, 2, 4, or a combination thereof. In certain embodiments, at least one of the nucleotide substitutions is a nucleotide substitution at position 2 of the modified 5′-UTR. In some particular embodiments, the nucleotide substitution at position 2 of the modified 5′-UTR is a U->G substitution.

In one embodiment, the nucleic acid molecule disclosed herein is a modified replicon RNA that comprises a modified 5′-UTR and is devoid of at least a portion of a nucleic acid sequence encoding viral structural proteins. In another embodiment, the modified 5′-UTR comprises one or more nucleotide substitutions at position 1, 2, 4, or a combination thereof. In another embodiment, the nucleotide substitution at position 2 of the modified 5′-UTR is a U->G substitution. In yet another embodiment, the replicon RNA comprises one or more expression cassettes, wherein each of the expression cassettes comprises a promoter operably linked to a heterologous nucleic acid sequence. In one embodiment, the modified replicon RNA (a) exhibits at least 80% sequence identity to the nucleic acid sequence of SEQ ID NO: 25, wherein the modified replicon RNA comprises a U->G substitution at position 2 of the 5′-untranslated region (5′-UTR) and is devoid of at least a portion of the sequence encoding viral structural proteins; or (b) comprises a 5′-UTR exhibiting at least 80% sequence identity to the nucleic acid sequence of at least one of SEQ ID NOs: 26-42 and a U->G substitution at position 2 of the 5′-UTR, and wherein the modified replicon RNA is devoid of at least a portion of the sequence encoding viral structural proteins.

Some viruses have sequences capable of forming one or more stem-loop structures which regulate, for example increase, capsid gene expression. The term “viral capsid enhancer” is used herein to refer to a regulatory element comprising sequences capable of forming such stem-loop structures. In some examples, the stem-loop structures are formed by sequences within the coding sequence of a capsid protein and named Downstream Loop (DLP) sequence. As disclosed herein, these stem-loop structures or variants thereof can be used to regulate, for example increase, expression level of genes of interest. For example, these stem-loop structures or variants thereof can be used in a recombinant vector (e.g., in a heterologous viral genome) for enhancing transcription and/or translation of coding sequence operably linked downstream thereto. As an example, members of the Alphavirus genus can resist the activation of antiviral RNA-activated protein kinase (PKR) by means of a prominent RNA structure present within in viral 26S transcripts, which allows an eIF2-independent translation initiation of these mRNAs. This structure, called the downstream loop (DLP), is located downstream from the AUG in SINV 26S mRNA and in other members of the Alphavirus genus. In the case of Sindbis virus, the DLP motif is found in the first ˜150 nt of the Sindbis subgenomic RNA. The hairpin is located downstream of the Sindbis capsid AUG initiation codon (AUG is collated at nt 50 of the Sindbis subgenomic RNA). Previous studies of sequence comparisons and structural RNA analysis revealed the evolutionary conservation of DLP in SINV and predicted the existence of equivalent DLP structures in many members of the Alphavirus genus (see e.g., Ventoso, J. Virol. 9484-9494, Vol. 86, September 2012).

PKR phosphorylates the eukaryotic translation initiation factor 2α (eIF2 α). Phosphorylation of eIF2 α blocks translation initiation of mRNA and in doing so keeps viruses from a completing a productive replication cycle. PKR is activated by interferon and double stranded RNA. Alphavirus replication in host cells is known to induce the double-stranded RNA-dependent protein kinase (PKR). For example, Sindbis virus infection of cells induces PKR that results in phosphorylation of eIF2 α yet the viral subgenomic mRNA is efficiently translated while translation of all other cellular mRNAs is restricted. The subgenomic mRNA of Sindbis virus has a stable RNA hairpin loop located downstream of the wild type AUG initiator codon for the virus capsid protein (e.g., capsid enhancer). This hairpin loop, also called stem-loop, RNA structure is often referred to as the Downstream LooP structure (or DLP motif). It has been reported that the DLP structure can stall a ribosome on the wild type AUG and this supports translation of the subgenomic mRNA without the requirement for functional eIF2 α. Thus, subgenomic mRNAs of Sindbis virus (SINV) as well as of other alphaviruses are efficiently translated even in cells that have highly active PKR resulting in complete phosphorylation of eIF2α.

The DLP structure was first characterized in Sindbis virus (SINV) 26S mRNA and also detected in Semliki Forest virus (SFV). Similar DLP structures have been reported to be present in at least 14 other members of the Alphavirus genus including New World (for example, MAYV, UNAV, EEEV (NA), EEEV (SA), AURAV) and Old World (SV, SFV, BEBV, RRV, SAG, GETV, MIDV, CHIKV, and ONNV) members. The predicted structures of these Alphavirus 26S mRNAs were constructed based on SHAPE (selective 2′-hydroxyl acylation and primer extension) data (Toribio et al., Nucleic Acids Res. May 19; 44(9):4368-80, 2016), the content of which is hereby incorporated by reference. Stable stem-loop structures were detected in all cases except for CHIKV and ONNV, whereas MAYV and EEEV showed DLPs of lower stability (Toribio et al., 2016 supra). The highest DLP activities were reported for those Alphaviruses that contained the most stable DLP structures. In some instances, DLP activity depends on the distance between the DLP motif and the initiation codon AUG (AUGi). The AUG-DLP spacing in Alphavirus 26S mRNAs is tuned to the topology of the ES6S region of the ribosomal 18S rRNA in a way that allows the placement of the AUGi in the P site of the 40S subunit stalled by the DLP, allowing the incorporation of Met-tRNA without the participation of eIF2. Two main topologies were detected: a compact and stable structure in the SFV clade, and a more extended structure in the SINV group. In both cases, it was observed that DLP structures were preceded by a region of intense SHAPE reactivity, suggesting a single stranded conformation for the AUG-DLP stretch. Accordingly, this region showed a high content of A and a low content of G that resulted in a low propensity to form secondary structures when compared with equivalent positions in whole mouse mRNA transcriptome or in those Alphavirus mRNAs lacking DLPs. These results reported by Toribio et al. (2016, supra) suggest that the occurrence of DLPs in Alphavirus is probably linked to a flattening of the preceding region, resulting in a valley-peak topology for this region of mRNA.

In the case of Sindbis virus, the DLP motif is found in the first ˜150 nt of the Sindbis subgenomic RNA. The hairpin is located downstream of the Sindbis capsid AUG initiation codon (AUG at nt 50 of the Sindbis subgenomic RNA) and results in stalling a ribosome such that the correct capsid gene AUG is used to initiate translation. This is because the hairpin causes ribosomes to pause eIF2α is not required to support translation initiation. Without being bound by any particular theory, it is believed that placing the DLP motif upstream of a coding sequence for any GOI typically results in a fusion-protein of N-terminal capsid amino acids that are encoded in the hairpin region to the GOI encoded protein because initiation occurs on the capsid AUG not the GOI AUG. In some embodiments disclosed herein, a porcine teschovirus-1 2A (P2A) peptide sequence was engineered in-frame immediately after the DLP sequence and in-frame immediately upstream of all GOI. The incorporation of the P2A peptide in the modified viral RNA replicons of the present disclosure allows release of a nearly pristine GOI protein from the capsid-GOI fusion; a single proline residue is added to all GOI proteins.

Without being bound by any particular theory, it is believed that the DLP allows translation to occur in an eIF2α independent manner, nucleic acid molecules and expression vectors (e.g., RNA replicon vectors) engineered to use it to initiate translation of non-structural proteins have increased functionality in cells that are innate immune system activated. Therefore, it is contemplated that DLP-engineered nucleic acid molecules and expression vectors (e.g., RNA replicon vectors) also function with more uniformity in different cells, individuals or populations of individuals because differences in the level of innate immune activation in each will naturally cause variability. In some embodiments, the DLP can assist in removing that variability because translation and replication of RNA replicon vectors (as well as GOI expression) can be less impacted by pre-existing innate immune responses. One of the significant values of the compositions and methods disclosed herein is that vaccine efficacy can be increased in individuals that are in a chronic or acute state of immune activation. Causes of chronic or acute immune activation could be found in individuals suffering from a subclinical or clinical infection or individuals undergoing medical treatments for cancer or other maladies (e.g., diabetes, malnutrition, high blood pressure, heart disease, Crohn's disease, muscular scleroses, etc.).

As described herein, DLP-containing nucleic acid molecules (for example, transcription and expression vectors (e.g., RNA viral replicons)) disclosed herein can be useful in conferring a resistance to the innate immune system in a subject. Unmodified RNA replicons are sensitive to the initial innate immune system state of cells they are introduced into. If the cells/individuals are in a highly active innate immune system state, the RNA replicon performance (e.g., replication and expression of a GOI) can be negatively impacted. By engineering a DLP to control initiation of protein translation, particularly of non-structural proteins, the impact of the pre-existing activation state of the innate immune system to influence efficient RNA replicon replication is removed or lessened. The result is more uniform and/or enhanced expression of a GOI that can impact vaccine efficacy or therapeutic impact of a treatment.

Since innate immune activation can occur due to many different stimuli, vaccine approaches that rely on self-amplifying RNA replicons to express antigen or therapeutic GOI can be negatively impacted by the global host protein shutdown associated with PKR phosphorylation of eIF2α. Engineering RNA replicons to function in a cellular environment where host protein translation is repressed would provide those systems with a significant advantage over standard RNA replicon systems.

Accordingly, RNA replicon systems that are negatively impacted by innate immune responses, such as systems derived from alphaviruses and arteriviruses, can be more effective at expressing their encoded GOI when engineered to contain a DLP motif. The DLP motif confers efficient mRNA translation in cellular environments where cellular mRNA translation is inhibited. When a DLP is linked with translation of a replicon vectors non-structural protein genes the replicase and transcriptase proteins are capable of initiating functional replication in PKR activated cellular environments. When a DLP is linked with translation of subgenomic mRNAs robust GOI expression is possible even when cellular mRNA is restricted due to innate immune activation. Accordingly, engineering replicons that contain DLP structures to help drive translation of both non-structural protein genes and subgenomic mRNAs provides yet another powerful way to overcome innate immune activation.

Some embodiments of the disclosure relate to DLP structures that have been engineered to support translation of viral non-structural genes of replicon vectors derived from two different viruses, Venezuelan equine encephalitis virus (VEEV) and equine arteritis virus (EAV), thus conveying innate immune response evasion to the systems. As described in greater detail below, incorporation of the DLP structures into the replicon vectors made them both resistant to interferon (IFN) treatment and unexpectedly also resulted in an overall increase in GOI expression potential. The combination of IFN resistance and superior protein expression potential imparted by engineering a DLP into the RNA replicon systems make them suitable for use in individuals or populations where innate immune activation is acutely or chronically present.

Some aspects of the present disclosure relate to nucleic acid molecules, such as synthetic or recombinant nucleic acid molecules, that include one or more DLP motifs, a coding sequence for one or more DLP motifs, or a combination thereof. In some embodiments, the nucleic acid molecules of the disclosure can include a coding sequence for a gene of interest (GOI) operably linked to DLP motif(s) and/or the coding sequence for the DLP motifs.

In one aspect, disclosed herein is a nucleic acid molecule, comprising (i) a first nucleic acid sequence encoding one or more structural elements of a viral capsid enhancer or a variant thereof and (ii) a second nucleic acid sequence operably linked to the first nucleic acid sequence, wherein the second nucleic acid sequence comprises a coding sequence for a gene of interest (GOI). In some embodiments, at least one of the one or more structural elements of the viral capsid enhancer comprises one or more RNA stem-loops. In some embodiments, at least one of the one or more RNA stem-loops is comprised by a DLP motif present in the first nucleic acid sequence. In some embodiments, at least one of the one or more structural elements of the viral capsid enhancer does not comprise any RNA stem-loop.

As described above, a viral capsid enhancer comprises sequences within the 5′ non-coding and/or 5′ coding sequences (preferably, the 5′ coding sequences) of that enhance expression (e.g., transcription and/or translation) of sequences operably linked therewith. In some embodiments of the present disclosure, the one or more structural elements of the viral capsid enhancer include one or two RNA stem-loops of the viral capsid enhancer. In some embodiments, the viral capsid enhancer of the present disclosure includes the sequences containing the 26S subgenomic promoter. In some embodiments, the viral capsid enhancer of the disclosure contains the 5′ coding sequences at about nucleotides 20 to 250, about nucleotides 20 to 200, about nucleotides 20 to 150, about nucleotides 20 to 100, or about nucleotides 50 to 250, about nucleotides 100 to 250, about nucleotides 50 to 200, about nucleotides 75 to 250, about nucleotides 75 to 200, about nucleotides 75 to 150, about nucleotides 77 to 139, or about nucleotides 100 to 250, about nucleotides 150 to 250, about nucleotides 100 to 150, about nucleotides 100 to 200 of the viral 26S RNA, which is capable of forming a hairpin structure. In some embodiments, the first nucleic acid sequence encoding one or more structural elements of a viral capsid enhancer that are important for enhancing expression of a heterologous sequence operably linked thereto. In some embodiments, the first nucleic acid sequence includes encoding sequence for one or more RNA stem-loops of a viral capsid enhancer. In some embodiments, the first nucleic acid sequence encoding one or more structural elements of a viral capsid enhancer that are important for enhancing translation of a heterologous sequence operably linked thereto. In some embodiments, the first nucleic acid sequence encoding one or more structural elements of a viral capsid enhancer that are important for enhancing transcription of a heterologous sequence operably linked thereto.

In some embodiments, the first nucleic acid sequence of the nucleic acid molecule includes at least about 50, about 75, about 100, about 150, about 200, about 300, or more nucleotides from the 5′ coding sequence for a viral capsid protein. In some embodiments, the first nucleic acid sequence of the nucleic acid molecule includes about 50, about 75, about 100, about 150, about 200, about 300, or more, or a range between any two of these values, nucleotides from the 5′ coding sequence for a viral capsid protein. In some embodiments, the viral capsid enhancer is derived from a capsid gene of an alphavirus species selected from the group consisting of Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), and Buggy Creek virus. In some embodiments, the viral capsid enhancer is derived from a capsid gene of a Sindbis virus species or a Semliki Forest virus species. In some particular embodiments, the viral capsid enhancer is derived from a capsid gene of a Sindbis virus species. Additionally, one of ordinary skill in the art will appreciate that modifications may be made in the 5′ coding sequences from the viral capsid protein without substantially reducing its enhancing activities. More information in this regard can be found in, e.g., Frolov et al., J. Virology 70:1182, 1994; Frolov et al., J. Virology 68:8111, 1994. In some embodiments, it can be advantage for such mutations to substantially preserve the RNA hairpin structure formed by the 5′ capsid coding sequences.

In some embodiments, the viral capsid enhancer disclosed herein does not contain one or more, or all, of the 5′ coding sequences of the capsid protein that are upstream of the hairpin structure. In some embodiments, the viral capsid enhancer disclosed herein does not contain all of the 5′ coding sequences of the viral capsid protein that are upstream of the hairpin structure. In some embodiments, the viral capsid enhancer sequence may encode all or part of the capsid protein. Accordingly, in some embodiments disclosed herein, the capsid enhancer region will not encode the entire viral capsid protein. In some embodiments, the viral capsid enhancer sequence encodes an amino terminal fragment from the viral capsid protein. In those embodiments in which an otherwise functional capsid protein is encoded by the capsid enhancer sequence, it may be desirable to ablate the capsid autoprotease activity. Capsid mutations that reduce or ablate the autoprotease activity of the capsid protein are known in the art (see e.g., WO1996/37616). In addition or alternatively, one or more of amino acid residues in the capsid protein may be altered to reduce capsid protease activity.

As indicated above, previous studies of sequence comparisons and structural RNA analysis revealed the evolutionary conservation of DLP motifs in many members of the Alphavirus genus (see e.g., Ventoso, 2012 supra). Accordingly, in some further embodiments, the viral capsid enhancer sequence of the present disclosure can be of any other variant sequence such as, for example, a synthetic sequence or a heterologous sequence, that can form an RNA hairpin functionally or structurally equivalent to one or more of the RNA stem-loops predicted for a viral capsid enhancer and which can act to enhance translation of RNA sequences operably linked downstream thereto (e.g., coding sequence for a gene of interest). In some embodiments, the nucleic acid molecule of the disclosure includes an alphavirus capsid enhancer as derived from Sindbis virus (SINV; NC 001547.1), Aura virus (AURAV; AF126284), Chikungunya virus (CHIKV; NC 004162), O'Nyong-Nyong virus (ONNV; NC 001512), Eastern Equine Encephalitis virus (EEEV(SA); AF159559 and EEEV (NA); U01558), Mayaro virus (MAYV; DQ001069), Semliki Forest virus (SFV; NC 003215), Ross River virus (RRV; DQ226993 and Sagiyama virus (SAGV; AB032553), Getah virus (GETV; NC 006558), Middelburg virus (MIDV; EF536323), Una virus (UNAV; AF33948), or Bebaru virus (BEBV; AF339480) as described in Toribio et al., 2016 supra, the content of which is hereby incorporated by reference in its entirety, or a variant thereof.

Nucleic acid molecules having a high degree of sequence identity (e.g., at least 85%, at least 90%, at least 95%, at least 96%, at least 97%, at least 98%, at least 99%, or 100% sequence identity) to the coding sequence for a viral capsid enhancer disclosed herein can be identified and/or isolated by using the sequence described herein (e.g., SEQ ID NO: 43) or any others alphavirus capsid protein as they are known in the art, for example, by using the sequences of Sindbis virus (SINV; NC 001547.1), Aura virus (AURAV; AF126284), Chikungunya virus (CHIKV; NC 004162), O'Nyong-Nyong virus (ONNV; NC 001512), Eastern Equine Encephalitis virus (EEEV(SA); AF159559 and EEEV (NA); U01558), Mayaro virus (MAYV; DQ001069), Semliki Forest virus (SFV; NC 003215), Ross River virus (RRV; DQ226993 and Sagiyama virus (SAGV; AB032553), Getah virus (GETV; NC 006558), Middelburg virus (MIDV; EF536323), Una virus (UNAV; AF33948), and Bebaru virus (BEBV; AF339480), by genome sequence analysis, hybridization, and/or PCR with degenerate primers or gene-specific primers from sequences identified in the respective alphavirus genome. For example, the viral capsid enhancer can comprise, or consist of, a DLP motif from a virus species belonging to the Togaviridae family, for example an alphavirus species or a rubivirus species. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the 5′ CDS portion of an alphavirus capsid protein. In some embodiments, the 5′ CDS portion of an alphavirus capsid protein comprises at least the first 25, 50, 75, 80, 100, 150, or 200 nucleotides of the coding sequence for the alphavirus capsid protein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the nucleic acid sequence of any one of SEQ ID NOs: 43-50. In some embodiments, the nucleic acid molecule comprises a viral capsid enhancer having a nucleic acid sequence that exhibits 80%, 85%, 90%, 91%, 92%, 93%, 94%, 95%, 96%, 97%, 98%, 99%, 100%, or a range between any two of these values, sequence identity to the nucleic acid sequence of any one of SEQ ID NOs: 43-50. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence of SEQ ID NO: 43 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to any one of the sequences described in the publication by Toribio et al. (2016 supra), the content of which is hereby incorporated by reference in its entirety.

Accordingly, in some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence of any one of SEQ ID NOs: 44-50 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 44 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 45 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 46 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 47 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 48 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 49 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 50 disclosed herein.

In the nucleic acid molecule according to some embodiments of the present disclosure, the one or more RNA stem-loops are operably positioned upstream of the coding sequence for the GOI of the second nucleic acid sequence. In some embodiments, the one or more RNA stem-loops are operably positioned from about 1 to about 50 nucleotides, from about 10 to about 75 nucleotides, from about 30 to about 100 nucleotides, from about 40 to about 150 nucleotides, from about 50 to about 200 nucleotides, from about 60 to about 250 nucleotides, from about 100 to about 300 nucleotides, or from about 150 to about 500 nucleotides upstream of the coding sequence for the GOI. In some embodiments, the one or more RNA stem-loops are operably positioned from about 1, about 2, about 5, about 10, about 15, about 20, about 25, about 30, about 40, about 50, about 60, about 70, about 80, about 90, about 100, about 200, about 300, about 400, about 500, or a range between any two of these values, nucleotides upstream of the coding sequence for the GOI. In some embodiments, the one or more RNA stem-loops are operably positioned immediately upstream of the coding sequence for the GOI.

In some embodiments, the nucleic acid molecule of the disclosure further includes a coding sequence for an autoprotease peptide (e.g., autocatalytic self-cleaving peptide), where the coding sequence for the autoprotease is optionally operably linked upstream to the second nucleic acid sequence. Generally, any proteolytic cleavage site known in the art can be incorporated into the nucleic acid molecules of the disclosure and can be, for example, proteolytic cleavage sequences that are cleaved post-production by a protease. Further suitable proteolytic cleavage sites also include proteolytic cleavage sequences that can be cleaved following addition of an external protease. As used herein the term “autoprotease” refers to a “self-cleaving” peptide that possesses autoproteolytic activity and is capable of cleaving itself from a larger polypeptide moiety. First identified in the foot-and-mouth disease virus (FMDV), a member of the picornavirus group, several autoproteases have been subsequently identified such as, for example, “2A like” peptides from equine rhinitis A virus (E2A), porcine teschovirus-1 (P2A) and Thosea asigna virus (T2A), and their activities in proteolytic cleavage have been shown in various ex vitro and in vivo eukaryotic systems. As such, the concept of autoproteases is available to one of skill in the art with many naturally-occurring autoprotease systems have been identified. Well studied autoprotease systems are e.g. viral proteases, developmental proteins (e.g. HetR, Hedgehog proteins), RumA autoprotease domain, UmuD, etc.). Non-limiting examples of autoprotease peptides suitable for the compositions and methods of the present disclosure include the peptide sequences from porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof.

In some embodiments, the coding sequence for an autoprotease peptide is operably linked downstream to the first nucleic acid sequence and upstream to the second nucleic acid sequence. In some embodiments, the autoprotease peptide comprises, or consists of, a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In some embodiments, the autoprotease peptide includes a peptide sequence of porcine teschovirus-1 2A (P2A).

One of skill in the art will appreciate that different configurations of the viral capsid enhancer sequence, the sequence encoding the autoprotease peptide, and the sequence encoding the gene of interest can be employed as long as the capsid enhancer sequence enhances expression of the heterologous nucleic acid sequence(s), e.g. a coding sequence for a GOI, as compared with the level seen in the absence of the capsid enhancer sequence. These sequences will typically be configured so that the polypeptide encoded by the gene of interest can be released from the protease and any capsid protein sequence after cleavage by the autoprotease.

Without being bound by any particular theory, it is believed that translational enhancing activity of a viral DLP motif can depend, in some embodiments, on the distance between the viral DLP motif and the initiation AUGi codon (Toribio et al., 2016 supra). Accordingly, in some embodiments, the first nucleic acid sequence is operably positioned a region of about 10 to 100 nucleotides downstream of the initiation codon AUGi of the modified viral RNA replicon. In some embodiments, the first nucleic acid sequence is operably positioned within a region of about 10 to 75, about 10 to 50, about 10 to 25, 15 to 75, about 15 to 50, about 15 to 25, about 25 to 75, about 25 to 50, about 25 to 100 nucleotides downstream of the initiation codon AUGi of the modified viral RNA replicon. In some embodiments, the first nucleic acid sequence is operably positioned within a region of about 25, 28, 31, 34, 37, 37, 40, 43, 46, 49, 50, or a range between any two of these values, nucleotides downstream of the initiation codon AUGi of the modified viral RNA replicon.

In some embodiments, the nucleic acid molecule as disclosed herein can further comprise a third nucleic acid sequence encoding one or more structural elements of a second viral capsid enhancer (e.g., a DLP motif), wherein the third nucleic acid sequence is operably linked upstream to the coding sequence for the GOI. The second DLP motif may be the same or may be different from the first DLP motif positioned upstream of the coding sequence for the nonstructural proteins. Accordingly, in some embodiments, the second DLP motif is the same as the first DLP motif positioned upstream of the coding sequence for the nonstructural proteins. In some embodiments, the second DLP motif is different from the first DLP motif positioned upstream of the coding sequence for the nonstructural proteins.

In some embodiments where the introduced nucleic acid molecule is a vector such as, for example, an RNA replicon, new mRNA copies may be generated which includes coding sequence for a gene of interest operably linked to one or more DLP motifs. The incorporation the one or more DLP motifs into the vector, e.g., RNA replicon, can then confer the intended enhancement of gene expression once the DLP-containing vector or replicon is introduced into the cells.

In one embodiment, the current disclosure is a nucleic acid molecule, comprising: a first nucleic acid sequence encoding one or more RNA stem-loops of a viral capsid enhancer or a variant thereof; and a second nucleic acid sequence operably linked to the first nucleic acid sequence, wherein the second nucleic acid sequence comprises a coding sequence for a gene of interest (GOI). In another aspect, the first nucleic acid sequence is operably linked upstream to the coding sequence for the GOI. In another aspect, the nucleic acid molecule further comprises a coding sequence for an autoprotease peptide operably linked upstream to the second nucleic acid sequence; the coding sequence for the autoprotease peptide is operably linked downstream to the first nucleic acid sequence and upstream to the second nucleic acid sequence; and the autoprotease peptide comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In another aspect, the viral capsid enhancer is derived from a capsid gene of a virus species belonging to the

Togaviridae family, wherein the viral capsid enhancer comprises a downstream loop (DLP) motif of the virus species, and the DLP motif comprises at least one of the one or more RNA stem-loops. In another aspect, the viral capsid enhancer comprises a nucleic acid sequence exhibiting at least 80% sequence identity to at least one of SEQ ID NOs: 43-50. In another aspect, the nucleic acid molecule further comprises a third nucleic acid sequence encoding one or more RNA stem-loops of a second viral capsid enhancer or a variant thereof; and a fourth nucleic acid sequence operably linked to the third nucleic acid sequence, wherein the fourth nucleic acid sequence comprises a coding sequence for a second gene of interest (GOI). The nucleic acid molecule can be a messenger RNA (mRNA) molecule or an RNA replicon. In another aspect, the nucleic acid molecule comprises a nucleic acid sequence encoding a modified viral RNA replicon, wherein the modified viral RNA replicon comprises: a first nucleic acid sequence encoding one or more structural elements of a viral capsid enhancer or a variant thereof, wherein the viral capsid enhancer is derived from a first virus species, and a second nucleic acid sequence derived from a second virus species encoding at least one nonstructural viral protein or a portion thereof, wherein the first nucleic acid sequence is operably linked upstream to the second nucleic acid sequence. The viral capsid enhancer comprises a downstream loop (DLP) motif of the first virus species, and wherein the DLP motif comprises at least one of the one or more RNA stem-loops. The viral capsid enhancer comprises a nucleic acid sequence exhibiting at least 80% sequence identity to at least one of SEQ ID NOs: 43-50.

In some embodiments, RNA replicons useful for the invention contain an RNA sub-sequence encoding a heterologous protein or peptide, such as the HBV antigens, and RNA sub-sequences encoding amino acid sequences derived from New World alphavirus nsP1, nsP2, and nsP4 proteins. The replicons also have an RNA sub-sequence encoding an amino acid sequence derived from an alphavirus nsP3 macro domain, and an RNA sub-sequence encoding an amino acid sequence derived from an alphavirus nsP3 central domain. The RNA replicons of the invention can also have an RNA sub-sequence encoding an amino acid sequence derived entirely from an Old World alphavirus nsP3 hypervariable domain; or can have an amino acid sequence having a portion derived from a New World alphavirus nsP3 hypervariable domain, and a portion derived from an Old World alphavirus nsP3 hypervariable domain. i.e. the HVD can be a hybrid or chimeric New World/Old World sequence.

The nsP1, nsP2, nsP3, and nsP4 proteins encoded by the replicon are functional or biologically active proteins. The RNA replicons of the invention can also encode a 3′ untranslated region (UTR) and a 5′ UTR, which can be alphavirus 3′ and 5′ UTRs. The RNA replicons can also encode control elements (e.g. one or more sub-genomic promoters) and a poly-A tail. The promoter, 5′ and/or 3′ UTRs, and RNA sub-sequence encoding the heterologous protein or peptide can be operably linked so that the replicon RNA self-amplifies and the heterologous protein or peptide is expressed in the organism.

The present inventors discovered that, unexpectedly, in an RNA replicon derived from a New World alphavirus genome, if at least a portion of the RNA encoding the nsP3 protein is substituted with RNA encoding at least a portion of nsP3 derived from an Old World alphavirus (OW), then the immunogenicity in a mammal to a heterologous protein or peptide encoded in the replicon is significantly reduced or eliminated. Thus, in some embodiments of the replicon the nsP3 macro domain and central domain can be derived from New World alphavirus sequences, while the HVD a) is derived from an Old World alphavirus HVD sequence, or b) has a portion derived from an Old World alphavirus HVD sequence and a portion derived from a New World alphavirus HVD sequence.

In another embodiment the macro and central domains are derived from Old World alphavirus macro and central domain sequences, and the HVD a) is derived from an Old World alphavirus HVD sequence, or b) has a portion derived from an Old World alphavirus HVD sequence and a portion derived from a New World alphavirus HVD sequence.

In another embodiment the macro domain is derived from a New World alphavirus macro domain sequence, the central domain is derived from an Old World alphavirus central domain sequence, and the HVD a) is derived from an Old World alphavirus HVD sequence, or b) has a portion derived from an Old World alphavirus HVD sequence and a portion derived from a New World alphavirus HVD sequence.

In another embodiment the macro domain is derived from an Old World alphavirus macro domain sequence, the central domain is derived from a New World alphavirus central domain sequence, and the HVD a) is derived from an Old World alphavirus HVD sequence, or b) has a portion derived from an Old World alphavirus HVD sequence and a portion derived from a New World alphavirus HVD sequence.

In some embodiments the replicon encodes an HVD that is a hybrid or chimeric New World/Old World sequence having a portion derived from a New World alphavirus HVD sequence and a portion derived from an Old World HVD sequence. In various embodiments the Old World portion can be at least 5 or at least 10 or at least 15 or at least 20 or at least 25 or at least 30 or at least 52 or at least 53 or at least 75 or at least 100 or at least 125 or at least 150 or at least 175 or at least 200 amino acids. The portions together can comprise an HVD having the same length as a wild type Old World or New World alphavirus HVD sequence, or can be up to 10 or up to 20 or up to 30 amino acids shorter; or can be up to 10 or up to 20 or up to 30 or up to 40 or up to 50 or up to 60 or up to 70 or up to 80 or up to 90 or up to 100 amino acids longer than a wild type, Old World or New World alphavirus HVD sequence.

In some embodiments the N-terminal portion of the HVD can be derived from the New World nsP3 HVD sequence and the C-terminal amino acids of the HVD can be derived from a wild type OW alphavirus HVD amino acid sequence, for example the at least 5 or at least 10 or at least 15 or at least 20 or at least 25 or at least 30 or at last 31 or at least 32 or at least 33 or at least 34 or at least 35 or 35-55 or 35-65 or at least 40 or at least 45 or at least 50 or at least 52 or at least 53 or at least 60 or at least 70 or at least 80 or at least 100 or at least 125 or at least 150 or at least 175 C-terminal amino acids of the HVD can be an amino acid sequence derived from (and optionally corresponding to) the amino acids of the OW HVD; in any of these embodiments the HVD can also be less than 200 or less than 175 or less than 150 or less than 125 or less than 100 or less than 80 amino acids in length. In further embodiments the C-terminal amino acids can be retained from the NW alphavirus C-terminal HVD sequence, such as the terminal 1-5 or 5 or 5-10 or 10-12 or 10-13 or 10-15 or 15-20 amino acids, while the remaining C-terminal amino acids can be derived from an OW alphavirus HVD as described.

In any of the embodiments described herein the New World alphavirus can be VEEV or EEEV or WEEV or any New World alphavirus described herein, and the Old World alphavirus can be CHIKV, SINV, or SFV or any Old World virus described herein. New World and Old World alphaviruses can be used in the invention in any combination, and all possible combinations and sub-combinations are disclosed as if set forth fully herein.

Alphaviruses are classified in the Group IV Togaviridae family of viruses. These viruses carry a positive-sense single-stranded RNA genome, which typically ranges from 11 kb-12 kb. The alphavirus replicons of the invention can be 11 kb-12 kb in length, or 10-13 kb, or 7-20 kb or 7-25 kb in length, and can have a 5′ cap and a 3′ poly-A tail, which can be an alphavirus 5′ cap and 3′ poly-A tail. The 5′ cap can be those known to persons of skill in the art, e.g. a 7-methylguanylate cap, or the anti-reverse cap analog 3′-O-Me-m7G(5′)ppp(5′)G or another analog cap structures. They are generally enveloped viruses and are spherical in shape, having a diameter of about 70 nm. They also can have an isometric nucleocapsid. The replicons can be encoded on a single piece of RNA. The alphavirus genome and the replicons have two open reading frames (ORFs), non-structural and structural. The non-structural portion of the genome encodes proteins nsP1-nsP4, which play a role in transcription and replication of viral RNA and are produced as a polyprotein and are the virus replication machinery. But the replicons can have one or two or more than two open reading frames. Any of the alphavirus replicons of the invention can lack, or not comprise, or not be comprised within or associated with, a capsid, nucleocapsid, coat protein, or nucleoprotein. The alphavirus replicons can be an RNA molecule.

The structural portion of the genome encodes the core nucleocapsid protein C, and envelope proteins P62 and E1 that associate as a heterodimer. The RNA replicons of the invention can have any one or more of the described characteristics of an alphavirus. In some embodiments the RNA replicons of the invention lack sequences encoding alphavirus structural proteins; or do not encode alphavirus (or, optionally, any other) structural proteins. In some embodiments the RNA replicons of the invention do not encode any one or more of protein C, P62, 6K, and E1, including all combinations and sub-combinations as if set forth fully herein. In some embodiments the RNA replicons of the invention do not encode any one of protein C, P62, 6K, and E1.

The geographic separation of the alphavirus family may be a factor in the evolution and adaption of these viruses to their unique environments. Circulating alphavirus sero-complexes can be further categorized as either Old World or New World alphaviruses. Old World and New World alphaviruses have sequences that can be utilized in the invention as described herein. New World alphaviruses include any New World alphavirus, for example the Eastern equine encephalitis virus (EEEV), the Venezuelan equine encephalitis virus (VEEV), Western equine encephalitis virus (WEEV), Fort Morgan (FMV), Highland J virus (HJV), Buggy Creek virus (BCRV), Mucambo virus (MUCV), and Pixuna virus (PIXV). The Old World alphaviruses include any Old World alphavirus, for example Sindbis virus (SINV), Semliki Forest virus (SFV), Chikungunya virus (CHIKV), Bebaru virus (BEBV), O'Nyong Nyong virus (ONNV), Ross River virus (RRV), Sagiyama virus (SAGV), Getah virus (GETV), Middleburg virus (MIDV), Ndumu virus (NDUV), Barmah Forest virus (BFV), Mayaro virus (MAYV), Aura virus (AURA), Una virus, Whataroa virus, Babank virus, and Kyzylagach virus. New World and Old World viruses and their sequences can be used in any combination or sub-combination in the RNA replicons of the invention, and are disclosed in all possible combinations and sub-combinations as if set forth fully herein.

The RNA replicons of the invention can be derived from alphavirus genomes, meaning that they have some of the structural characteristics of alphavirus genomes, or be similar to them. The RNA replicons of the invention can be modified alphavirus genomes. In some embodiments of the replicons disclosed herein one or more sequences of the replicon can be provided “in trans,” i.e. the sequences of the replicon are provided on more than one RNA molecule. In other embodiments all of the sequences of the replicon are present on a single RNA molecule, which can also be administered to a mammal to be treated as described herein.

The RNA replicons of the invention can contain RNA sequences from (or amino acid sequences encoded by) a wild-type New World or Old World alphavirus genome. Any of the RNA replicons of the invention disclosed herein can contain RNA sequences “derived from” or “based on” wild type alphavirus genome sequences, meaning that they have at least 60% or at least 65% or at least 68% or at least 70% or at least 80% or at least 85% or at least 90% or at least 95% or at least 97% or at least 98% or at least 99% or 100% or 80-99% or 90-100% or 95-99% or 95-100% or 97-99% or 98-99% sequence identity with an RNA sequence (which can be a corresponding RNA sequence) from a wild type RNA alphavirus genome, which can be a New World or Old World alphavirus genome. Any of the nucleic acids or amino acid sequences disclosed herein can be functional or biologically active and operably linked to another sequence required for self-replication of the alphavirus or replicon. A molecule is functional or biologically active if it performs at least 50% of the same activity as its natural (or wild type), corresponding molecule, but a functional molecule can also perform at least 60% or at least 70% or at least 90% or at least 95% or 100% of the same activity as its natural (or wild type) corresponding molecule. The RNA replicons can also encode an amino acid sequence derived from or based on a wild type alphavirus amino acid sequence, meaning that they have at least 60% or at least 65% or at least 68% or at least 70% or at least 80% at least 70% or at least 80% or at least 90% or at least 95% or at least 97% or at least 98% or at least 99% or 100% or 80-99% or 90-100% or 95-99% or 95-100% or 97-99% or 98-99% sequence identity with an amino acid sequence (which can be a corresponding sequence) encoded by a wild type RNA alphavirus genome, which can be a New World or Old World alphavirus genome. Sequences derived from other sequences can be up to 5% or up to 10% or up to 20% or up to 30% longer or shorter than the original sequence. In any of the embodiments the sequence identity can be at least 95% or at least 97% or at least 98% or at least 99% or 100% for any nucleotide sequence encoding (or amino acid sequence having) a G3BP or FXR binding site thereon. These sequences can also be up to 5% or up to 10% or up to 20% or up to 30% longer or shorter than the original sequence.

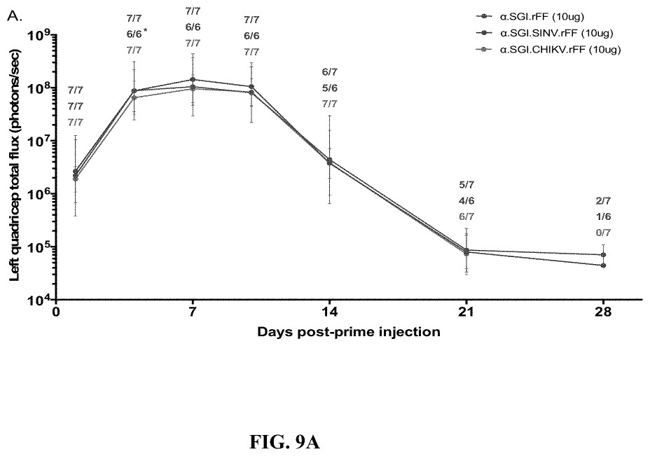

For example, in some embodiments the RNA sequences encoding any one or more of the nsP1, nsP2, nsP3 macro domain, nsP3 central domain, nsP3 hypervariable domain, and/or nsP4 proteins, can be derived from corresponding wild type alphavirus sequences. The “corresponding” sequence can be the analogous sequence in another type of alphavirus. Corresponding sequences are disclosed herein and can also be determined through sequence alignment tools known to persons of ordinary skill (e.g. Clustal Omega). FIG. 7 shows a sequence alignment illustrating the corresponding sequences of nsP3 proteins from representative members of Old World and New World alphaviruses, which was obtained using Clustal Omega. But other sequence alignment tools accepted by persons of ordinary skill in the art can also be used. Programs useful for performing sequence alignments are also found in Molecular Systems Biology (2011) 7, 539. Thus, nsP1, nsP2, nsP3, nsP4 sequences from a New World alphavirus “correspond to” nsP1, nsP2, nsP3, nsP4 sequences from an Old World alphavirus, respectively. Sub-sequences can be corresponding sequences as well. Corresponding amino acid sequences can be at least 5 or at least 10 or at least 15 or at least 20 or at least 25 or at least 30 or at least 52 or at least 53 or at least 75 or at least 100 or at least 125 or at least 150 or at least 175 or at least 200 amino acids, and up to 5% or up to 10% or up to 20% or up to 30% longer or shorter than the original sequence; corresponding nucleic acid sequences can be at least 15 or at least 30 or at least 45 or at least 60 or at least 75 or at least 90 or at least 156 or at least 159 or at least 225 or at least 300 or at least 375 or at least 450 or at least 525 or at least 600 nucleotides. Such sequences can be up to 5% or up to 10% or up to 20% or up to 30% longer or shorter than the original sequence.

In some embodiments of the replicons each of the nsP1, nsP2, and nsP4 sequences can be derived from or based on a New World alphavirus genome. In some embodiments the RNA replicon derived from or based on a wild type New World alphavirus genome can contain at least one RNA sequence (besides at least one heterologous protein or peptide) that is not from a wild type New World alphavirus genome, which can be the sequence of nsP3, or of a central and/or macro domain(s) of nsP3, or of at least a portion of the HVD. In some embodiments the RNA replicon derived from a New World alphavirus genome can have an RNA sequence encoding nsP3, or a domain of nsP3, or a portion of a domain of nsP3 substituted with a corresponding sequence from a wild type Old World alphavirus genome. When referring to the whole replicon “derived from” or “based on” does not count the sub-sequence(s) of RNA that encode(s) the at least one heterologous protein or peptide and, optionally, can also not count the sequence encoding the nsP3 protein, or any one or more the macro domain, the central domain, and/or the HVD domain of nsP3 in any combination or sub-combination.

The term “RNA replicon” refers to RNA which contains all of the genetic information required for directing its own amplification or self-replication within a permissive cell, which can be a human, mammalian, or animal cell. The RNA replicon 1) encodes an RNA-dependent RNA polymerase, which may interact with viral or host cell-derived proteins, nucleic acids or ribonucleoproteins to catalyze the RNA amplification process. The non-structural proteins include nsP1, nsP2, nsP3, nsP4; and 2) contains cis-acting RNA sequences required for replication and transcription of the genomic and subgenomic RNAs, such as 3′ and 5′ UTRs (alphavirus nucleotide sequences for non-structural protein-mediated amplification), and/or a sub-genomic promoter. These sequences may be bound during the process of replication to self-encoded proteins, or non-self-encoded cell-derived proteins, nucleic acids or ribonucleoproteins, or complexes between any of these components. In some embodiments, a modified RNA replicon molecule typically contains the following ordered elements: 5′ viral RNA sequence(s) required in cis for replication (e.g. a 5′ UTR and a 5′ CSE), sequences coding for biologically active nonstructural proteins (e.g. nsP1234), a promoter for transcribing the subgenomic RNA, 3′ viral sequences required in cis for replication (e.g. 3′ UTR), and a polyadenylate tract, and optionally, a sequence (or two or more sequences) encoding a heterologous protein or peptide after or under the control of a sub-genomic promoter. Further, the term RNA replicon can refer to a positive sense (or message sense) molecule and the RNA replicon can be of a length different from that of any known, naturally-occurring RNA viruses. In any of the embodiments of the present disclosure, the RNA replicon can lack (or not contain) the sequence(s) of at least one (or all) of the structural viral proteins (e.g. nucleocapsid protein C, and envelope proteins P62, 6K, and E1). In these embodiments, the sequences encoding one or more structural genes can be substituted with one or more heterologous sequences such as, for example, a coding sequence for at least one heterologous protein or peptide (or other gene of interest (GOI)).

In various embodiments the RNA replicons disclosed herein can be engineered, synthetic, or recombinant RNA replicons. As used herein, the term recombinant means any molecule (e.g. DNA, RNA, etc.), that is or results, however indirectly, from human manipulation of a polynucleotide. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant RNA replicon can be one or more of the following: 1) synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules; 2) conjoined nucleotide sequences that are not conjoined in nature; 3) engineered using molecular cloning techniques such that it lacks one or more nucleotides with respect to the naturally occurring nucleotide sequence; and 4) manipulated using molecular cloning techniques such that it has one or more sequence changes or rearrangements with respect to the naturally occurring nucleotide sequence.

In disclosing the nucleic acid or polypeptide sequences herein, for example sequences of nsP1, nsP2, nsP3, nsP3 macro domain, nsP3 central domain, nsP3 hypervariable domain, nsP4, RdRp, P1234, also disclosed are sequences considered to be based on or derived from the original sequence. Sequences disclosed therefore include polypeptide sequences having sequence identities of at least 40%, at least 45%, at least 50%, at least 55%, of at least 60%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or 100% or 85-99% or 85-95% or 90-99% or 95-99% or 97-99% or 98-99% sequence identity with the full-length polypeptide sequence of any of SEQ ID NOs: 51-67 (and nucleotide sequences encoding any of SEQ ID NOs: 51-67), and fragments thereof. Also disclosed are fragments or portions of any of the sequences disclosed herein. Fragments or portions of sequences can include sub-sequences having at least 5 or at least 7 or at least 10, or at least 20, or at least 30, at least 50, at least 75, at least 100, at least 125, 150 or more or 5-10 or 10-12 or 10-15 or 15-20 or 20-40 or 20-50 or 30-50 or 30-75 or 30-100 amino acid residues of the entire sequence (or a nucleic acid encoding such fragments), or at least 100 or at least 200 or at least 300 or at least 400 or at least 500 or at least 600 or at least 700 or at least 800 or at least 900 or at least 1000 or 100-200 or 100-500 or 100-1000 or 500-1000 amino acid residues (or a nucleic acid encoding such fragments), or any of these amounts but less than 500 or less than 700 or less than 1000 or less than 2000 consecutive amino acids of any of SEQ ID NOs: 51-67 or of any fragment disclosed herein, or a nucleic acid encoding such fragments. Also disclosed are variants of such sequences, e.g., where at least one or two or three or four or five amino acid residue has been inserted N- and/or C-terminal to, and/or within, the disclosed sequence(s) which contain(s) the insertion and substitution, and nucleic acid sequences encoding such variants. Contemplated variants can additionally or alternately include those containing predetermined mutations by, e.g., homologous recombination or site-directed or PCR mutagenesis, and the corresponding polypeptides or nucleic acids of other species, including, but not limited to, those described herein, the alleles or other naturally occurring variants of the family of polypeptides or nucleic acids which contain an insertion and substitution; and/or derivatives wherein the polypeptide has been covalently modified by substitution, chemical, enzymatic, or other appropriate means with a moiety other than a naturally occurring amino acid which contains the insertion and substitution (for example, a detectable moiety such as an enzyme). The nucleic acid sequences described herein can be RNA sequences.

Any of the components or sequences of the RNA replicon can be operably linked to any other of the components or sequences. The components or sequences of the RNA replicon can be operably linked for the expression of the at least one heterologous protein or peptide (or biotherapeutic) in a host cell or treated organism and/or for the ability of the replicon to self-replicate. The term “operably linked” denotes a functional linkage between two or more sequences that are configured so as to perform their usual function. Thus, a promoter or UTR operably linked to a coding sequence is capable of effecting the transcription and expression of the coding sequence when the proper enzymes are present. The promoter need not be contiguous with the coding sequence, so long as it functions to direct the expression thereof. Thus, an operable linkage between an RNA sequence encoding a heterologous protein or peptide and a regulatory sequence (for example, a promoter or UTR) is a functional link that allows for expression of the polynucleotide of interest. Operably linked can also refer to sequences such as the sequences encoding the RdRp (e.g. nsP4), nsP1-4, the UTRs, promoters, and other sequences encoding in the RNA replicon, are linked so that they enable transcription and translation of the biotherapeutic molecule and/or replication of the replicon. The UTRs can be operably linked by providing sequences and spacing necessary for recognition and translation by a ribosome of other encoded sequences.

Alphavirus genomes encode non-structural proteins nsP1, nsP2, nsP3, and nsP4, which are produced as a single polyprotein precursor, sometimes designated P1234 (or nsP1-4 or nsP1234), and which is cleaved into the mature proteins through proteolytic processing. nsP1 can be about 60 kDa in size and may have methyltransferase activity and be involved in the viral capping reaction. nsP2 has a size of about 90 kDa and may have helicase and protease activity while nsP3 is about 60 kDa and contains three domains: a macrodomain, a central (or alphavirus unique) domain, and a hypervariable domain (HVD). nsP4 is about 70 kDa in size and contains the core RNA-dependent RNA polymerase (RdRp) catalytic domain. After infection the alphavirus genomic RNA is translated to yield a P1234 polyprotein, which is cleaved into the individual proteins.

Alphavirus nsP3 protein contains three domains; a) a macro domain, b) a central (or alpha) domain, and c) a hypervariable domain (HVD). In various embodiments the replicons of the invention have an RNA sequence encoding an nsP3 macro domain derived from a wild type alphavirus nsP3, and an nsP3 central domain derived from a wild type alphavirus nsP3. In various embodiments the macro and central domain(s) can both be derived from a New World wild type alphavirus nsP3, or can both be derived from an Old World wild type alphavirus nsP3 protein. In more embodiments the macro domain can be derived from a New World wild type alphavirus macro domain and the central domain can be derived from an Old World wild type alphavirus central domain, or vice versa. The various domains can be of any sequence described herein.

In some embodiments the replicons can have a New World alphavirus HVD where the sequence to the C-terminal side of the amino acid where an FXR binding site begins can be deleted and replaced with a replacement sequence of an Old World wild type alphavirus HVD sequence, or portion thereof. Old World alphavirus replacement sequences are described herein. Thus, when the New World alphavirus is VEEV, those amino acids to the C-terminal side of amino acid 478 can be deleted; when the New World alphavirus is EEEV, those amino acids to the C-terminal side of amino acid 531 can be deleted; and when the New World alphavirus is WEEV, those amino acids to the C-terminal side of amino acid 504 can be deleted. In any of these embodiments a replacement sequence can be substituted as described herein. As otherwise described herein, a portion of the C-terminal amino acids of the New World alphavirus HVD can be nevertheless retained at the C-terminal side of the Old World sequence.

In some embodiments at least a portion of the sequence encoding the FXR binding site in the New World alphavirus can be deleted and replaced with a replacement sequence, which are described herein. Thus, when the NW alphavirus is VEEV amino acids 478-517 or 478-545 can be deleted and replaced with a replacement sequence of an OW alphavirus. Or when the NW alphavirus is VEEV at least one of the repeats present between amino acids 478-545 can be deleted and optionally replaced with an OW alphavirus replacement sequence. When the NW alphavirus is EEEV amino acids 531-547 can be deleted and replaced with a replacement sequence. When the NW alphavirus is WEEV amino acids 504-520 can be deleted and replaced with a replacement sequence. In other embodiments the entire sequence encoding the FXR binding site can be deleted, or at least 50% or at last 70% or at least 80% or at least 90% of the FXR binding site can be deleted, and optionally replaced with a replacement sequence. In any of the embodiments the indicated sequence can be deleted and no replacement sequence inserted.

The OW alphavirus replacement sequences can comprise amino acids fragments having one or more G3BP binding sites, or at least a portion of a G3BP binding site. Thus, a replacement sequence can be FGDF or FGSF. A replacements sequence can also be derived from at least a portion of a wild type nsP3 hypervariable domain of an Old World alphavirus. Further examples of OW alphavirus replacement sequences are described below. The OW alphavirus replacement sequences can be used in replicons having sequences of any of the New World alphavirus HVD sequences described herein. In any of the embodiments the New World alphavirus can be VEEV, EEEV, WEEV, or any New World alphavirus described herein.

When the OW alphavirus is CHIKV, the replacement sequence can be amino acids 479-582 or 479-500 or 479-500 of CHIKV nsP3.

When the OW alphavirus is SINV, the replacement sequence can be a sequence comprising amino acids 490-493 or 513-516 or 490-516 of SINV nsP3.

When the OW alphavirus is SFV the replacement sequence can be a sequence comprising amino acids 451-471, or 451-454, or 468-471 of SFV nsP3.

When the OW alphavirus is MAYV, the replacement sequence can be a sequence comprising amino acids 470-473 of MAYV nsP3.

When the OW alphavirus is RRV, the replacement sequence can be a sequence comprising amino acids 412-426, or 512-515, or 523-526 of RRV nsP3.

When the OW alphavirus is ONNV, the replacement sequence can be a sequence comprising amino acids 519-540, or 519-522, or 537-540 of ONNV nsP3.

When the OW alphavirus is BFV, the replacement sequence can be a sequence comprising amino acids 429-450, or 429-432, or 447-450 of BFV nsP3.

The New World and Old World alphaviruses can be any described herein and can be combined in any possible combination or sub-combination, all of which are disclosed as if set forth fully herein.

The alphavirus genome encodes a core RNA-dependent RNA polymerase in nsP4. Cleavage of the polyprotein may occur at the nsP2/3 junction, influencing the RNA template used during genome replication. After cleavage nsP3 may create a ring structure that encircles nsP2, and these two proteins have a substantial interface. Thus, preservation of the sequences around the junctions of nsP2/3 and/or nsP3/4 may be useful.

Thus, in some embodiments the macro and/or central and/or HVD domains of the nsP3 protein can have the C-terminal and/or the N-terminal portions (as described herein) being an amino acid sequence derived from a New World alphavirus while the remaining portion of the domain(s) is/are derived from an Old World alphavirus sequence. For example, the macro and/or central and/or HVD domains can have a sequence derived from a corresponding Old World alphavirus domain but have the first 4 or 5 or 6 or 4-6 or 6-8 or 6-10 amino acids of the N-terminal and/or the C-terminal of nsP3 being derived from the New World alphavirus sequence (which can be the New World alphavirus from which the nsP1, nsP2, and nsP4 are derived). Thus, the replicon can be as described herein having an RNA sub-sequence encoding an amino acid sequence derived from an Old World alphavirus nsP3 macro and/or central and/or HVD domain and the first 1-3 or 1-4 or 1-5 or 1-6 or 1-7 or 1-8 amino acids on the N-terminal and/or C-terminal side of the domain(s) are derived from a New World alphavirus domain(s), or may have one or two or three substitutions thereon. As used in this context the term “C-terminal” and “N-terminal” do not indicate a true terminus, but the point at which the nsPs will be cleaved into separate polypeptides. The sequences encoding the non-specific proteins (nsPs) feature a stop codon and normally transcription will stop at that point. But when the stop codon is treated as a readthrough stop codon the terminus can be the “/” indicated in SEQ ID NOs: 12-17, which can represent the N-terminal and/or C-terminal of the nsPs. The junction sequences can be those 1-6 amino acids on either side of the terminus, e.g. on the nsP3 side. These embodiments allow the nsP3 sequence to be derived from Old World sequences yet preserve the junctions between nsP2/nsP3, and between nsP3/nsP4. The preservation of these junctions may permit cleavage of the P1234 protein junctions using the New World alphavirus enzymes. In some embodiments the penultimate glycine is preserved in the junction. The Old World alphavirus can be any described herein. For example, when the New World alphavirus is VEEV, the nsP2/nsP3 sequence can be (SEQ ID NO: 62) LHEAGC/APSY, with the slash (“/”) representing the border between nsP2 and nsP3, and with the penultimate G preserved while the remaining amino acids in the nsP2/nsP3 junction are varied as described herein. In the case of the nsP3/nsP4 junction of VEEV, the sequence can be (SEQ ID NO: 63) RFDAGA/YIFS, with the penultimate glycine again preserved and the remaining nsP3 amino acids varied as described herein. These sequences can also be preceded by a stop codon (TGA), which as noted above can sometimes be treated as a readthrough stop codon. When the New World alphavirus is EEEV, the nsP2/nsP3 sequence can be (SEQ ID NO: 64) QHEAGR/APAY, with the slash (“/”) representing the border between nsP2 and nsP3, and with the penultimate G preserved while the remaining amino acids in the nsP2/nsP3 junction are varied as described herein. In the case of the nsP3/nsP4 junction of EEEV, the sequence can be (SEQ ID NO: 65) RYEAGA/YIFS, with the penultimate glycine again preserved and the remaining nsP3 amino acids varied as described herein. These sequences can also be preceded by a read-through stop codon (TGA), as above. When the New World alphavirus is WEEV, the nsP2/nsP3 sequence can be (SEQ ID NO: 66) RYEAGR/APAY, with the slash (“/”) representing the end or terminus of nsP2 (and the junction between nsP2 and nsP3), and with the penultimate G preserved while the remaining amino acids in the nsP2/nsP3 junction are varied as described herein. In the case of the nsP3/nsP4 junction of WEEV, the sequence can be (SEQ ID NO: 67) RYEAGA/YIFS, with the penultimate glycine again preserved and the remaining nsP3 amino acids varied as described herein. These sequences can also be preceded by a read-through stop codon (TGA), as explained herein. Any of these sequences (SEQ ID NOs: 62-67) can also contain one or two or three substitutions on the N-terminal and/or C-terminal sides.

Alphaviruses can contain conserved sequence elements (CSEs), which are similar or identical sub-sequences in nucleic acid sequences or polypeptides across species. The CSEs can occur in the HVD of a New World or Old World alphavirus nsP3 and are known in the art.

Old World alphaviruses can also contain FGDF or FGSF amino acid motifs, which can repeat in the sequence to form a repeat sequence or repeating motif. In any of the embodiments of the RNA replicon of the invention the HVD of the OW alphavirus can contain an FGDF/FGDF repeat, or an FGSF/FGSF repeat, or an FGDF/FGSF repeat, or an FGSF/FGDF repeat. In all embodiments where a repeat is present the two repeating motifs can be separated by one or more amino acid residues. In various embodiments the two repeating motifs can be separated by 5 or 6 or 7 or 8 or 9 or 10 or at least 10 or 11 or 12 or 13 or 14 or 15 or 16 or 17 or 18 or 19 or 20 or 21 or 22 or 23 or 24 or 25 amino acid residues or by more than 25 amino acid residues, which in one embodiment can be random amino acids. In one embodiment the motifs or repeat motifs are separated by at least 10 and not more than 25 amino acids, which also can be random amino acids. In various embodiments the two repeating motifs can be separated by SEQ ID No: 56: NEGEIESLSSELLT, or SEQ ID NO: 57: SDGEIDELSRRVTTESEPVL, or SEQ ID NO: 58: DEHEVDALASGIT, or a sequence derived from any of them, which can be of the same length; thus, disclosed are repeating motifs separated by SEQ ID NOs: 56, 57, or 58 having 1) an FGDF motif on both ends; 2) an FGSF motif on both ends; 3) an FGDF motif on either the 3′ or 5′ end and an FGSF motif on the opposite end. In various embodiments amino acid sequences can also follow the second motif. Examples include the amino acid sequence SEQ ID NO: 61: DDVLRLGRAGA or SEQ ID NO: 60: EPGEVNSIISSRSAVSFPLRKQRRRRRSRRTEY or SEQ ID NO: 59: LPGEVDDLTDSDWSTCSDTDDELRLDRAGG, or a sequence derived from any of them, any of which can follow a motif or repeating motif disclosed herein.

Any of the replicons of the invention can also comprise a 5′ and a 3′ untranslated region (UTR). The UTRs can be wild type New World or Old World alphavirus UTR sequences, or a sequence derived from any of them. In various embodiments the 5′ UTR can be of any suitable length, such as about 60 nt or 50-70 nt or 40-80 nt. In some embodiments the 5′ UTR can also have conserved primary or secondary structures (e.g. one or more stem-loop(s)) and can participate in the replication of alphavirus or of replicon RNA. In some embodiments the 3′ UTR can be up to several hundred nucleotides, for example it can be 50-900 or 100-900 or 50-800 or 100-700 or 200 nt — 700 nt. The ‘3 UTR also can have secondary structures, e.g. a step loop, and can be followed by a polyadenylate tract or poly-A tail. In any of the embodiments of the invention the 5’ and 3′ untranslated regions can be operably linked to any of the other sequences encoded by the replicon. The UTRs can be operably linked to a promoter and/or sequence encoding a heterologous protein or peptide by providing sequences and spacing necessary for recognition and transcription of the other encoded sequences.

In one embodiment the RNA replicon of the invention can have an RNA sequence encoding a heterologous protein or peptide (e.g. a monoclonal antibody or a biotherapeutic protein or peptide), RNA sequences encoding amino acid sequences derived from a wild type New World alphavirus nsP1, nsP2, and nsP4 protein sequences, and 5′ and 3′ UTR sequences (for non-structural protein-mediated amplification). The RNA replicons can also have a 5′ cap and a polyadenylate (or poly-A) tail. The RNA replicon can also encode an amino acid sequence derived from a New World alphavirus macro domain, an amino acid sequence derived from a New World alphavirus central domain, and an amino acid sequence derived from an Old World alphavirus hypervariable domain. In alternative embodiments the RNA replicon can encode a portion having an amino acid sequence derived from a New World hypervariable domain, and another portion having an amino acid sequence derived from an Old World alphavirus hypervariable domain, as described herein.

The immunogenicity of a heterologous protein or peptide can be determined by a number of assays known to persons of ordinary skill, for example immunostaining of intracellular cytokines or secreted cytokines by epitope-specific T-cell populations, or by quantifying frequencies and total numbers of epitope-specific T-cells and characterizing their differentiation and activation state, e.g. short-lived effector and memory precursor effector CD8+ T-cells. Immunogenicity can also be determined by measuring an antibody-mediated immune response, e.g. the production of antibodies by measuring serum IgA or IgG titers.

The RNA replicon in the current disclosure can have the following aspects:
Aspect 1. An RNA replicon comprising an RNA sub-sequence encoding a heterologous protein or peptide; 5′ and 3′ alphavirus untranslated regions; RNA sub-sequences encoding amino acid sequences derived from New World alphavirus nonstructural proteins nsP1, nsP2, and nsP4; and an RNA sub-sequence encoding an amino acid sequence derived from an alphavirus nsP3 macro domain; an RNA sub-sequence encoding an amino acid sequence derived from an alphavirus nsP3 central domain; and an RNA sub-sequence encoding a hypervariable domain comprising
a. an amino acid sequence derived from an Old World alphavirus nsP3 hypervariable domain; or
b. an amino acid sequence comprising a portion derived from a New World alphavirus nsP3 hypervariable domain, and a portion derived from an Old World alphavirus nsP3 hypervariable domain.
Aspect 2. The RNA replicon of aspect 1 wherein the alphavirus nsP3 macro domain and the alphavirus nsP3 central domain are from a New World alphavirus.
Aspect 3. The RNA replicon of aspect 1 wherein the alphavirus nsP3 macro domain and the alphavirus nsP3 central domain are from an Old World alphavirus.
Aspect 4. The RNA replicon of aspect 1 comprising the amino acid sequence derived from an Old World alphavirus nsP3 hypervariable domain.
Aspect 5. The RNA replicon of aspect 4 wherein the Old World alphavirus is selected from the group consisting of: CHIKV, SINV, and SFV.
Aspect 6. The RNA replicon of aspect 4 wherein the New World alphavirus is Venezuelan Equine Encephalitis Virus (VEEV).
Aspect 7. The RNA replicon of aspect 1 wherein the New World alphavirus is Venezuelan Equine Encephalitis Virus (VEEV).
Aspect 8. The RNA replicon of aspect 1 wherein the New World alphavirus is selected from the group consisting of: a Venezuelan equine encephalitis virus (VEEV), a western equine encephalitis virus (WEEV), and an eastern equine encephalitis virus (EEEV).
Aspect 9. The RNA replicon of aspect 8 wherein the Old World alphavirus is selected from the group consisting of: Sindbis virus (SINV), Chickungunya virus (CHIKV), Semliki Forest Virus (SFV), Ross River Virus (RRV), Sagiyama virus (SAGV), Getah virus (GETV), Middleburg virus (MIDV), Bebaru virus (BEBV), O'nyong nyong virus (ONNV), Ndumu (NDUV), and Barmah Forest virus (BFV).
Aspect 10. The RNA replicon of aspect 1 wherein the portion derived from the Old World alphavirus nsP3 hypervariable domain comprises a motif selected from the group consisting of: FGDF and FGSF.
Aspect 11. The RNA replicon of aspect 1 wherein the portion derived from the Old World alphavirus nsP3 hypervariable domain comprises a repeat selected from the group consisting of: an FGDF/FGDF repeat, an FGSF/FGSF repeat, an FGDF/FGSF repeat, and an FGSF/FGDF repeat; and further wherein the repeat sequences are separated by at least 10 and not more than 25 amino acids.
Aspect 12. The RNA replicon on aspect 11 wherein the repeat sequence is separated by an amino acid sequence derived from the group consisting of: SEQ ID NO: 56: NEGEIESLSSELLT and SEQ ID NO: 57: SDGEIDELSRRVTTESEPVL and SEQ ID NO: 58: DEHEVDALASGIT.

Aspect 13. The RNA replicon of aspect 10 wherein the portion derived from the Old World alphavirus hypervariable domain comprises

a. amino acids 479-482 or 497-500 or 479-500 or 335-517 of CHIKV nsP3 HVD; or

b. amino acids 451-454 or 468-471 or 451-471 of SFV nsP3 HVD; or

c. amino acids 490-493 or 513-516 or 490-516 or 335-538 of SINV nsP3 HVD.
Aspect 14. The RNA replicon of aspect 11 wherein the portion derived from the Old World alphavirus hypervariable domain comprises

a. amino acids 479-500 or 335-517 of CHIKV nsP3 HVD; or

b. amino acids 451-471 of SFV nsP3 HVD; or

c. amino acids 490-516 of SINV nsP3 HVD.
Aspect 15. The RNA replicon of aspect 13 wherein the New World alphavirus is VEEV and the portion derived from the New World alphavirus hypervariable domain does not comprise amino acids 478-518 of the VEEV nsP3 hypervariable domain.
Aspect 16. The RNA replicon of aspect 13 wherein the New World alphavirus is VEEV and the portion derived from the New World alphavirus hypervariable domain does not comprise amino acids 478-545 of the VEEV nsP3 hypervariable domain.
Aspect 17. The RNA replicon of aspect 13 wherein the New World alphavirus is VEEV and the portion derived from the New World alphavirus hypervariable domain does not comprise amino acids 335-518 of the VEEV nsP3 hypervariable domain.
Aspect 18. The RNA replicon of aspect 17 wherein the Old World alphavirus is CHIKV and the portion derived from the Old World alphavirus hypervariable domain comprises amino acids 335-517 of CHIKV.
Aspect 19. The RNA replicon of aspect 17 wherein the Old World alphavirus is SINV and the portion derived from the Old World alphavirus hypervariable domain comprises amino acids 335-538 of SINV.
Aspect 20. The RNA replicon of aspect 13 further comprising that the New World alphavirus is EEEV, and the portion derived from the New World alphavirus hypervariable domain does not comprise amino acids 531-547 of the EEEV hypervariable domain.
Aspect 21. The RNA replicon of aspect 20 wherein the New World alphavirus is EEEV, and the portion derived from the New World alphavirus hypervariable domain does not comprise amino acids 531-547 of the EEEV hypervariable domain, and wherein the portion derived from the Old World alphavirus hypervariable domain comprises
a. amino acids 479-500 of CHIKV nsP3 HVD; or
b. amino acids 451-471 of SFV nsP3 HVD; or
c. amino acids 490-516 of SINV nsP3 HVD.
Aspect 22. The RNA replicon of aspect 13 further comprising that the New World alphavirus is WEEV, and the portion derived from the New World alphavirus hypervariable domain does not comprise amino acids 504-520 of the WEEV hypervariable domain.
Aspect 23. The RNA replicon of aspect 22 wherein the New World alphavirus is WEEV, and the portion derived from the New World alphavirus hypervariable domain does not comprise amino acids 504-520 of the WEEV hypervariable domain, and wherein the portion derived from the Old World alphavirus hypervariable domain comprises
a. amino acids 479-500 of CHIKV nsP3 HVD; or
b. amino acids 451-471 of SFV nsP3 HVD; or
c. amino acids 490-516 of SINV nsP3 HVD.
Aspect 24. The RNA replicon of aspect 1 further comprising a sub-genomic promoter that is operably linked to and regulates translation of the RNA sequence encoding the heterologous protein.
Aspect 25. The RNA replicon of aspect 1 further comprising a 5′ cap and a 3′ poly-A tail.
Aspect 26. The RNA replicon of aspect 1 wherein the replicon comprises positive sense, single-stranded RNA.
Aspect 27. The RNA replicon of aspect 25, wherein the replicon comprises 10-12 kb of RNA and has a diameter of 30-50 nm.
Aspect 28. The RNA replicon of aspect 1 wherein the heterologous protein is a biotherapeutic protein or peptide.
Aspect 29. The RNA replicon of aspect 1 wherein the heterologous protein is an antibody.
Aspect 30. The RNA replicon of aspect 1 wherein the New World alphavirus is VEEV, and the portion derived from a New World alphavirus nsP3 hypervariable domain does not comprise amino acids 335-518 of the VEEV nsP3 hypervariable domain, and wherein the portion derived from an Old World alphavirus nsP3 hypervariable domain comprises amino acids 490-493 or 513-516 or 490-516 or 335-538 of SINV nsP3 HVD.
Aspect 31. The RNA replicon of aspect 30 wherein the portion derived from an Old World alphavirus nsP3 hypervariable domain comprises amino acids 490-516 of SINV nsP3 HVD.
Aspect 32. The RNA replicon of aspect 30 wherein the Old World alphavirus is SINV and the portion derived from an Old World alphavirus nsP3 hypervariable domain comprises amino acids 335-538 of SINV nsP3 HVD.
Aspect 33. The RNA replicon of aspect 1 wherein the RNA sequence encoding the heterologous protein or peptide is operably linked to the RNA sequence encoding the nsP1, nsP2, and nsP4.
Aspect 34. An RNA replicon comprising an RNA sub-sequence encoding a heterologous protein or peptide; RNA sub-sequences encoding amino acid sequences derived from New World alphavirus nonstructural proteins nsP1, nsP2, and nsP4; and an RNA sub-sequence encoding an amino acid sequence derived from an Old World alphavirus nsP3 protein, and wherein the first 1-6 amino acids on the N-terminal and/or C-terminal side of the nsP3 protein are derived from an New World alphavirus sequence.

A self-replicating RNA vector of the application can be a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection.

A self-replicating RNA replicon useful for the invention can comprise any regulatory elements to establish conventional function(s) of the vector, including but not limited to replication and expression of the HBV antigen(s) encoded by the polynucleotide sequence of the vector. Regulatory elements include, but are not limited to, a promoter, an enhancer, a polyadenylation signal, translation stop codon, a ribosome binding element, a transcription terminator, selection markers, origin of replication, etc. A vector can comprise one or more expression cassettes. An “expression cassette” is part of a vector that directs the cellular machinery to make RNA and protein. An expression cassette typically comprises three components: a promoter sequence, an open reading frame, and a 3′-untranslated region (UTR) optionally comprising a polyadenylation signal. An open reading frame (ORF) is a reading frame that contains a coding sequence of a protein of interest (e.g., HBV antigen) from a start codon to a stop codon. Regulatory elements of the expression cassette can be operably linked to a polynucleotide sequence encoding an HBV antigen of interest. As used herein, the term “operably linked” is to be taken in its broadest reasonable context and refers to a linkage of polynucleotide elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

A vector can comprise a promoter sequence, preferably within an expression cassette, to control expression of an HBV antigen of interest. The term “promoter” is used in its conventional sense and refers to a nucleotide sequence that initiates the transcription of an operably linked nucleotide sequence. A promoter is located on the same strand near the nucleotide sequence it transcribes. Promoters can be a constitutive, inducible, or repressible.

Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). Preferably, the promoter is located upstream of the polynucleotide encoding an HBV antigen within an expression cassette. For example, the promoter can be a subgenomic promoter for the alphavirus. The accumulated experimental evidence has demonstrated that replication/amplification of VEEV and other alphavirus genomes and their defective interfering (DI) RNAs is determined by three promoter elements: (i) the conserved 3′-terminal sequence element (3′ CSE) and the following poly(A) tail; (ii) the 5′ UTR, which functions as a key promoter element for both negative- and positive-strand RNA synthesis; and (iii) the 51-nt conserved sequence element (51-nt CSE), which is located in the nsP1-coding sequence and functions as an enhancer of alphavirus genome replication (Kim et al., PNAS, 2014, 111: 10708-10713, and references therein).

A vector can comprise additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a protein of interest (e.g., an HBV antigen) within an expression cassette of the vector. Enhancer sequences are regulatory DNA sequences that, when bound by transcription factors, enhance the transcription of an associated gene. An enhancer sequence is preferably located upstream of the polynucleotide sequence encoding an HBV antigen, but downstream of a promoter sequence within an expression cassette of the vector.

Any polyadenylation signal known to those skilled in the art in view of the present disclosure can be used. For example, the polyadenylation signal can be a SV40 polyadenylation signal, LTR polyadenylation signal, bovine growth hormone (bGH) polyadenylation signal, human growth hormone (hGH) polyadenylation signal, or human β-globin polyadenylation signal. Preferably, a polyadenylation signal is a bovine growth hormone (bGH) polyadenylation signal or a SV40 polyadenylation signal. A nucleotide sequence of an exemplary bGH polyadenylation signal is shown in SEQ ID NO: 20. A nucleotide sequence of an exemplary SV40 polyadenylation signal is shown in SEQ ID NO: 13.

Any enhancer sequence known to those skilled in the art in view of the present disclosure can be used. For example, an enhancer sequence can be human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein AI precursor (ApoAI), untranslated R-U5 domain of the human T-cell leukemia virus type 1 (HTLV-1) long terminal repeat (LTR), a splicing enhancer, a synthetic rabbit β-globin intron, or any combination thereof. Preferably, an enhancer sequence is a composite sequence of three consecutive elements of the untranslated R-U5 domain of HTLV-1 LTR, rabbit β-globin intron, and a splicing enhancer, which is referred to herein as “a triple enhancer sequence.” A nucleotide sequence of an exemplary triple enhancer sequence is shown in SEQ ID NO: 10. Another exemplary enhancer sequence is an ApoAI gene fragment shown in SEQ ID NO: 12.

A vector can comprise a polynucleotide sequence encoding a signal peptide sequence. Preferably, the polynucleotide sequence encoding the signal peptide sequence is located upstream of the polynucleotide sequence encoding an HBV antigen. Signal peptides typically direct localization of a protein, facilitate secretion of the protein from the cell in which it is produced, and/or improve antigen expression and cross-presentation to antigen-presenting cells. A signal peptide can be present at the N-terminus of an HBV antigen when expressed from the vector, but is cleaved off by signal peptidase, e.g., upon secretion from the cell. An expressed protein in which a signal peptide has been cleaved is often referred to as the “mature protein.” Any signal peptide known in the art in view of the present disclosure can be used. For example, a signal peptide can be a cystatin S signal peptide; an immunoglobulin (Ig) secretion signal, such as the Ig heavy chain gamma signal peptide SPIgG or the Ig heavy chain epsilon signal peptide SPIgE.

Preferably, a signal peptide sequence is a cystatin S signal peptide. Exemplary nucleic acid and amino acid sequences of a cystatin S signal peptide are shown in SEQ ID NOs: 8 and 9, respectively. Exemplary nucleic acid and amino acid sequences of an immunoglobulin secretion signal are shown in SEQ ID NOs: 14 and 15, respectively.

In a particular embodiment of the application, a self-replicating replicon comprises an expression cassette including a polynucleotide encoding at least one of an HBV antigen selected from the group consisting of an HBV pol antigen comprising an amino acid sequence at least 90%, such as 90%, 91%, 92%, 93%, 94%, 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical to SEQ ID NO: 7, and a truncated HBV core antigen consisting of the amino acid sequence at least 95%, such as 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical to SEQ ID NO: 2 or SEQ ID NO: 4; an upstream sequence operably linked to the polynucleotide encoding the HBV antigen comprising, from 5′ end to 3′ end, a promoter sequence, preferably a subgenomic promoter, and a polynucleotide sequence encoding a signal peptide sequence, preferably a cystatin S signal peptide having the amino acid sequence of SEQ ID NO: 9; and a downstream sequence operably linked to the polynucleotide encoding the HBV antigen comprising a polyadenylation signal, preferably a bGH polyadenylation signal of SEQ ID NO: 20. Such vector further comprises an expression cassette including a polynucleotide encoding replication proteins comprising one or more viral non-structural proteins (nsP1, nsP2, nsP3, and nspP4) that drive replication of the RNA replicon.

In an embodiment of the application, a self-replicating RNA molecule encodes an HBV Pol antigen having the amino acid sequence of SEQ ID NO: 7. Preferably, the self-replicating RNA molecule comprises a coding sequence for the HBV Pol antigen that is at least 90% identical to the polynucleotide sequence of SEQ ID NO: 5 or 6, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or 6, preferably 100% identical to SEQ ID NO: 5 or 6.

In an embodiment of the application, a self-replicating RNA molecule encodes a truncated HBV core antigen consisting of the amino acid sequence of SEQ ID NO: 2 or SEQ ID NO: 4. Preferably, the self-replicating RNA molecule comprises a coding sequence for the truncated HBV core antigen that is at least 90% identical to the polynucleotide sequence of SEQ ID NO: 1 or SEQ ID NO: 3, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 100% identical to SEQ ID NO: 1 or SEQ ID NO: 3.

In yet another embodiment of the application, a self-replicating RNA molecule encodes a fusion protein comprising an HBV Pol antigen having the amino acid sequence of SEQ ID NO: 7 and a truncated HBV core antigen consisting of the amino acid sequence of SEQ ID NO: 1 or SEQ ID NO: 3. Preferably, the self-replicating RNA molecule comprises a coding sequence for the fusion, which contains a coding sequence for the truncated HBV core antigen at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3, more preferably SEQ ID NO: 1 or SEQ ID NO: 3, operably linked to a coding sequence for the HBV Pol antigen at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6, more preferably SEQ ID NO: 5 or SEQ ID NO: 6. Preferably, the coding sequence for the truncated HBV core antigen is operably linked to the coding sequence for the HBV Pol antigen via a coding sequence for a linker at least 90% identical to SEQ ID NO: 11, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 11, preferably 98%, 99% or 100% identical to SEQ ID NO: 11. In particular embodiments of the application, a self-replicating RNA molecule comprises a coding sequence for the fusion having SEQ ID NO: 1 or SEQ ID NO: 3 operably linked to SEQ ID NO: 11, which is further operably linked to SEQ ID NO: 5 or SEQ ID NO: 6.

The polynucleotides and expression vectors encoding the HBV antigens of the application can be made by any method known in the art in view of the present disclosure. For example, a polynucleotide encoding an HBV antigen can be introduced or “cloned” into an expression vector using standard molecular biology techniques, e.g., polymerase chain reaction (PCR), etc., which are well known to those skilled in the art.

### Compositions, Therapeutic Combinations, and Vaccines

The application also relates to compositions, therapeutic combinations, more particularly kits, and vaccines comprising one or more HBV antigens, polynucleotides, and/or vectors encoding one or more HBV antigens according to the application. Any of the HBV antigens, polynucleotides, and/or vectors of the application described herein can be used in the compositions, therapeutic combinations or kits, and vaccines of the application.

In an embodiment of the application, a composition comprises a self-replicating RNA molecule comprising a polynucleotide encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4.

In an embodiment of the application, a composition comprises a self-replicating RNA molecule, comprising a polynucleotide encoding an HBV Pol antigen comprising an amino acid sequence that is at least 90% identical to SEQ ID NO: 7, preferably 100% identical to SEQ ID NO: 7.

In an embodiment of the application, a composition comprises a self-replicating RNA molecule, comprising a polynucleotide encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4; and a self-replicating RNA molecule, comprising a polynucleotide encoding an HBV Pol antigen comprising an amino acid sequence that is at least 90% identical to SEQ ID NO: 7, preferably 100% identical to SEQ ID NO: 7. The self-replicating RNA molecule comprising the coding sequence for the truncated HBV core antigen and the self-replicating RNA molecule comprising the coding sequence for the HBV Pol antigen can be the same self-replicating RNA molecule, or two different self-replicating RNA molecules.

In an embodiment of the application, a composition comprises a self-replicating RNA molecule, comprising a polynucleotide encoding a fusion protein comprising a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4, operably linked to an HBV Pol antigen comprising an amino acid sequence that is at least 90% identical to SEQ ID NO: 7, preferably 100% identical to SEQ ID NO: 7, or vice versa. Preferably, the fusion protein further comprises a linker that operably links the truncated HBV core antigen to the HBV Pol antigen, or vice versa. Preferably, the linker has the amino acid sequence of (AlaGly)n, wherein n is an integer of 2 to 5.

The application also relates to a therapeutic combination or a kit comprising a self-replicating RNA expressing a truncated HBV core antigen and an HBV pol antigen according to embodiments of the application. Any self-replicating RNA molecules encoding HBV core and pol antigens of the application described herein can be used in the therapeutic combinations or kits of the application.

In a particular embodiment of the application, a therapeutic combination or kit comprises a self-replicating RNA replicon comprising: i) a first polynucleotide sequence encoding a truncated HBV core antigen consisting of an amino acid sequence that is at least 95% identical to SEQ ID NO: 2; and ii) a second polynucleotide sequence encoding an HBV polymerase antigen having an amino acid sequence that is at least 90% identical to SEQ ID NO: 7, wherein the HBV polymerase antigen does not have reverse transcriptase activity and RNase H activity.

According to embodiments of the application, the polynucleotides in a vaccine combination or kit can be linked or separate, such that the HBV antigens expressed from such polynucleotides are fused together or produced as separate proteins, whether expressed from the same or different polynucleotides. In an embodiment, the first and second polynucleotides are present in separate vectors, e.g., RNA replicons, used in combination either in the same or separate compositions, such that the expressed proteins are also separate proteins, but used in combination. In another embodiment, the HBV antigens encoded by the first and second polynucleotides can be expressed from the same vector, e.g., such that an HBV core-pol fusion antigen is produced. Optionally, the core and pol antigens can be joined or fused together by a short linker. Alternatively, the HBV antigens encoded by the first and second polynucleotides can be expressed independently from a single vector using a using a ribosomal slippage site (also known as cis-hydrolase site) between the core and pol antigen coding sequences. This strategy results in a bicistronic expression vector in which individual core and pol antigens are produced from a single mRNA transcript. The core and pol antigens produced from such a bicistronic expression vector can have additional N or C-terminal residues, depending upon the ordering of the coding sequences on the mRNA transcript. Examples of ribosomal slippage sites that can be used for this purpose include, but are not limited to, the FA2 slippage site from foot-and-mouth disease virus (FMDV). Another possibility is that the HBV antigens encoded by the first and second polynucleotides can be expressed independently from two separate vectors, one encoding the HBV core antigen and one encoding the HBV pol antigen.

In a preferred embodiment, the first and second polynucleotides are present in separate self-replicating RNA molecules. Preferably, the separate self-replicating RNA molecules are present in the same composition.

According to preferred embodiments of the application, a therapeutic combination or kit comprises a first polynucleotide present in a first self-replicating RNA molecule, a second polynucleotide present in a second self-replicating RNA molecule. The first and second self-replicating RNA molecules can be the same or different.

In another preferred embodiment, the first and second polynucleotides are present in a single self-replicating RNA molecule.

When a therapeutic combination of the application comprises a first self-replicating RNA molecule, and a second self-replicating RNA molecule, the amount of each of the first and second self-replicating RNA molecule is not particularly limited. For example, the first self-replicating RNA molecule and the second self-replicating RNA molecule can be present in a ratio of 10:1 to 1:10, by weight, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, by weight. Preferably, the first and second self-replicating RNA molecules are present in a ratio of 1:1, by weight. The therapeutic combination of the application can further comprise a third vector encoding a third active agent useful for treating an HBV infection.

Compositions and therapeutic combinations of the application can comprise additional polynucleotides or vectors encoding additional HBV antigens and/or additional HBV antigens or immunogenic fragments thereof, such as an HBsAg, an HBV L protein or HBV envelope protein, or a polynucleotide sequence encoding thereof. However, in particular embodiments, the compositions and therapeutic combinations of the application do not comprise certain antigens.

In a particular embodiment, a composition or therapeutic combination or kit of the application does not comprise a HBsAg or a polynucleotide sequence encoding the HBsAg.

In another particular embodiment, a composition or therapeutic combination or kit of the application does not comprise an HBV L protein or a polynucleotide sequence encoding the HBV L protein.

In yet another particular embodiment of the application, a composition or therapeutic combination of the application does not comprise an HBV envelope protein or a polynucleotide sequence encoding the HBV envelope protein.

Compositions and therapeutic combinations of the application can also comprise a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. Pharmaceutically acceptable carriers can include vehicles, such as lipid nanoparticles (LNPs). The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Compositions and therapeutic combinations of the application can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Compositions of the application can also be formulated for other routes of administration including transmucosal, ocular, rectal, long acting implantation, sublingual administration, under the tongue, from oral mucosa bypassing the portal circulation, inhalation, or intranasal.

In a preferred embodiment of the application, compositions and therapeutic combinations of the application are formulated for parental injection, preferably subcutaneous, intradermal injection, or intramuscular injection, more preferably intramuscular injection.

According to embodiments of the application, compositions and therapeutic combinations for administration will typically comprise a buffered solution in a pharmaceutically acceptable carrier, e.g., an aqueous carrier such as buffered saline and the like, e.g., phosphate buffered saline (PBS). The compositions and therapeutic combinations can also contain pharmaceutically acceptable substances as required to approximate physiological conditions such as pH adjusting and buffering agents. For example, a composition or therapeutic combination of the application comprising a self-replicating RNA molecule can contain phosphate buffered saline (PBS) as the pharmaceutically acceptable carrier.

Compositions and therapeutic combinations of the application can be formulated as a vaccine (also referred to as an “immunogenic composition”) according to methods well known in the art. Such compositions can include adjuvants to enhance immune responses. The optimal ratios of each component in the formulation can be determined by techniques well known to those skilled in the art in view of the present disclosure.

In certain embodiments, a further adjuvant can be included in a composition or therapeutic combination of the application, or co-administered with a composition or therapeutic combination of the application. Use of another adjuvant is optional, and can further enhance immune responses when the composition is used for vaccination purposes. Other adjuvants suitable for co-administration or inclusion in compositions in accordance with the application should preferably be ones that are potentially safe, well tolerated and effective in humans. An adjuvant can be a small molecule or antibody including, but not limited to, immune checkpoint inhibitors (e.g., anti-PD1, anti-TIM-3, etc.), toll-like receptor agonists (e.g., TLR7 agonists and/or TLR8 agonists), RIG-1 agonists, IL-15 superagonists (Altor Bioscience), mutant IRF3 and IRF7 genetic adjuvants, STING agonists (Aduro), FLT3L genetic adjuvant, and IL-7-hyFc. For example, adjuvants can e.g., be chosen from among the following anti-HBV agents: HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, CTLA-4 inhibitors; Agonists of co-stimulatory receptors that are expressed on immune cells (more particularly T cells), such as CD27 and CD28; BTK inhibitors; Other drugs for treating HBV; IDO inhibitors; Arginase inhibitors; and KDM5 inhibitors.

In certain embodiments, each of the first and second non-naturally occurring nucleic acid molecules is independently formulated with a lipid nanoparticle (LNP).

The application also provides methods of making compositions and therapeutic combinations of the application. A method of producing a composition or therapeutic combination comprises mixing an isolated polynucleotide encoding an HBV antigen, vector, and/or polypeptide of the application with one or more pharmaceutically acceptable carriers. One of ordinary skill in the art will be familiar with conventional techniques used to prepare such compositions.

### Methods of Inducing an Immune Response or Treating an HBV Infection

The application also provides methods of inducing an immune response against hepatitis B virus (HBV) in a subject in need thereof, comprising administering to the subject an immunogenically effective amount of a composition or immunogenic composition of the application. Any of the compositions and therapeutic combinations of the application described herein can be used in the methods of the application.

As used herein, the term “infection” refers to the invasion of a host by a disease-causing agent. A disease-causing agent is considered to be “infectious” when it is capable of invading a host, and replicating or propagating within the host. Examples of infectious agents include viruses, e.g., HBV and certain species of adenovirus, prions, bacteria, fungi, protozoa and the like. “HBV infection” specifically refers to invasion of a host organism, such as cells and tissues of the host organism, by HBV.

The phrase “inducing an immune response” when used with reference to the methods described herein encompasses causing a desired immune response or effect in a subject in need thereof against an infection, e.g., an HBV infection. “Inducing an immune response” also encompasses providing a therapeutic immunity for treating against a pathogenic agent, e.g., HBV. As used herein, the term “therapeutic immunity” or “therapeutic immune response” means that the vaccinated subject is able to control an infection with the pathogenic agent against which the vaccination was done, for instance immunity against HBV infection conferred by vaccination with HBV vaccine. In an embodiment, “inducing an immune response” means producing an immunity in a subject in need thereof, e.g., to provide a therapeutic effect against a disease, such as HBV infection. In certain embodiments, “inducing an immune response” refers to causing or improving cellular immunity, e.g., T cell response, against HBV infection. In certain embodiments, “inducing an immune response” refers to causing or improving a humoral immune response against HBV infection. In certain embodiments, “inducing an immune response” refers to causing or improving a cellular and a humoral immune response against HBV infection.

As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all. Usually, a subject having a “protective immune response” or “protective immunity” against a certain agent will not die as a result of the infection with said agent.

Typically, the administration of compositions and therapeutic combinations of the application will have a therapeutic aim to generate an immune response against HBV after HBV infection or development of symptoms characteristic of HBV infection, e.g., for therapeutic vaccination.

As used herein, “an immunogenically effective amount” or “immunologically effective amount” means an amount of a composition, polynucleotide, vector, or antigen sufficient to induce a desired immune effect or immune response in a subject in need thereof. An immunogenically effective amount can be an amount sufficient to induce an immune response in a subject in need thereof. An immunogenically effective amount can be an amount sufficient to produce immunity in a subject in need thereof, e.g., provide a therapeutic effect against a disease such as HBV infection. An immunogenically effective amount can vary depending upon a variety of factors, such as the physical condition of the subject, age, weight, health, etc.; the particular application, e.g., providing protective immunity or therapeutic immunity; and the particular disease, e.g., viral infection, for which immunity is desired. An immunogenically effective amount can readily be determined by one of ordinary skill in the art in view of the present disclosure.

In particular embodiments of the application, an immunogenically effective amount refers to the amount of a composition or therapeutic combination which is sufficient to achieve one, two, three, four, or more of the following effects: (i) reduce or ameliorate the severity of an HBV infection or a symptom associated therewith; (ii) reduce the duration of an HBV infection or symptom associated therewith; (iii) prevent the progression of an HBV infection or symptom associated therewith; (iv) cause regression of an HBV infection or symptom associated therewith; (v) prevent the development or onset of an HBV infection, or symptom associated therewith; (vi) prevent the recurrence of an HBV infection or symptom associated therewith; (vii) reduce hospitalization of a subject having an HBV infection; (viii) reduce hospitalization length of a subject having an HBV infection; (ix) increase the survival of a subject with an HBV infection; (x) eliminate an HBV infection in a subject; (xi) inhibit or reduce HBV replication in a subject; and/or (xii) enhance or improve the prophylactic or therapeutic effect(s) of another therapy.

An immunogenically effective amount can also be an amount sufficient to reduce HBsAg levels consistent with evolution to clinical seroconversion; achieve sustained HBsAg clearance associated with reduction of infected hepatocytes by a subject's immune system; induce HBV-antigen specific activated T-cell populations; and/or achieve persistent loss of HBsAg within 12 months. Examples of a target index include lower HBsAg below a threshold of 500 copies of HBsAg international units (IU) and/or higher CD8 counts.

It is expected that the amount will fall in a relatively broad range that can be determined through routine trials. The RNA content of compositions of the invention will generally be expressed in terms of the amount of RNA per dose. For example, a dose can have ≤10 μg RNA, and expression can be seen at much lower levels e.g. ≤1 μg/dose, ≤100 ng/dose, ≤10 ng/dose, ≤1 ng/dose, etc.

An immunogenically effective amount can be from one vector, or from multiple vectors. As further general guidance, an immunogenically effective amount when used with reference to a peptide can range from about 10 μg to 1 mg per administration, such as 10, 20, 50, 100, 200, 300, 400, 500, 600, 700, 800, 9000, or 1000 μg per administration. An immunogenically effective amount can be administered in a single composition, or in multiple compositions, such as 1, 2, 3, 4, 5, 6, 7, 8, 9, or 10 compositions (e.g., tablets, capsules or injectables, or any composition adapted to intradermal delivery, e.g., to intradermal delivery using an intradermal delivery patch), wherein the administration of the multiple capsules or injections collectively provides a subject with an immunogenically effective amount. It is also possible to administer an immunogenically effective amount to a subject, and subsequently administer another dose of an immunogenically effective amount to the same subject, in a so-called prime-boost regimen. This general concept of a prime-boost regimen is well known to the skilled person in the vaccine field. Further booster administrations can optionally be added to the regimen, as needed.

A therapeutic combination comprising two self-replicating RNA molecules, e.g., a first self-replicating RNA molecule encoding an HBV core antigen and second self-replicating RNA molecule encoding an HBV pol antigen, can be administered to a subject by mixing both replicons and delivering the mixture to a single anatomic site. Alternatively, two separate immunizations each delivering a single expression replicon can be performed. In such embodiments, whether both replicons are administered in a single immunization as a mixture of in two separate immunizations, the first self-replicating RNA molecule and the second self-replicating RNA molecule can be administered in a ratio of 10:1 to 1:10, by weight, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, by weight. Preferably, the first and second self-replicating RNA molecules are administered in a ratio of 1:1, by weight.

Preferably, a subject to be treated according to the methods of the application is an HBV-infected subject, particular a subject having chronic HBV infection. Acute HBV infection is characterized by an efficient activation of the innate immune system complemented with a subsequent broad adaptive response (e.g., HBV-specific T-cells, neutralizing antibodies), which usually results in successful suppression of replication or removal of infected hepatocytes. In contrast, such responses are impaired or diminished due to high viral and antigen load, e.g., HBV envelope proteins are produced in abundance and can be released in sub-viral particles in 1,000-fold excess to infectious virus.

Chronic HBV infection is described in phases characterized by viral load, liver enzyme levels (necroinflammatory activity), HBeAg, or HBsAg load or presence of antibodies to these antigens. cccDNA levels stay relatively constant at approximately 10 to 50 copies per cell, even though viremia can vary considerably. The persistence of the cccDNA species leads to chronicity. More specifically, the phases of chronic HBV infection include: (i) the immune-tolerant phase characterized by high viral load and normal or minimally elevated liver enzymes; (ii) the immune activation HBeAg-positive phase in which lower or declining levels of viral replication with significantly elevated liver enzymes are observed; (iii) the inactive HBsAg carrier phase, which is a low replicative state with low viral loads and normal liver enzyme levels in the serum that can follow HBeAg seroconversion; and (iv) the HBeAg-negative phase in which viral replication occurs periodically (reactivation) with concomitant fluctuations in liver enzyme levels, mutations in the pre-core and/or basal core promoter are common, such that HBeAg is not produced by the infected cell.

As used herein, “chronic HBV infection” refers to a subject having the detectable presence of HBV for more than 6 months. A subject having a chronic HBV infection can be in any phase of chronic HBV infection. Chronic HBV infection is understood in accordance with its ordinary meaning in the field. Chronic HBV infection can for example be characterized by the persistence of HBsAg for 6 months or more after acute HBV infection. For example, a chronic HBV infection referred to herein follows the definition published by the Centers for Disease Control and Prevention (CDC), according to which a chronic HBV infection can be characterized by laboratory criteria such as: (i) negative for IgM antibodies to hepatitis B core antigen (IgM anti-HBc) and positive for hepatitis B surface antigen (HBsAg), hepatitis B e antigen (HBeAg), or nucleic acid test for hepatitis B virus DNA, or (ii) positive for HBsAg or nucleic acid test for HBV DNA, or positive for HBeAg two times at least 6 months apart.

Preferably, an immunogenically effective amount refers to the amount of a composition or therapeutic combination of the application which is sufficient to treat chronic HBV infection. In some embodiments, a subject having chronic HBV infection is undergoing nucleoside analog (NUC) treatment, and is NUC-suppressed. As used herein, “NUC-suppressed” refers to a subject having an undetectable viral level of HBV and stable alanine aminotransferase (ALT) levels for at least six months. Examples of nucleoside/nucleotide analog treatment include HBV polymerase inhibitors, such as entacavir and tenofovir. Preferably, a subject having chronic HBV infection does not have advanced hepatic fibrosis or cirrhosis. Such subject would typically have a METAVIR score of less than 3 for fibrosis and a fibroscan result of less than 9 kPa. The METAVIR score is a scoring system that is commonly used to assess the extent of inflammation and fibrosis by histopathological evaluation in a liver biopsy of patients with hepatitis B. The scoring system assigns two standardized numbers: one reflecting the degree of inflammation and one reflecting the degree of fibrosis.

It is believed that elimination or reduction of chronic HBV can allow early disease interception of severe liver disease, including virus-induced cirrhosis and hepatocellular carcinoma. Thus, the methods of the application can also be used as therapy to treat HBV-induced diseases. Examples of HBV-induced diseases include, but are not limited to cirrhosis, cancer (e.g., hepatocellular carcinoma), and fibrosis, particularly advanced fibrosis characterized by a METAVIR score of 3 or higher for fibrosis. In such embodiments, an immunogenically effective amount is an amount sufficient to achieve persistent loss of HBsAg within 12 months and significant decrease in clinical disease (e.g., cirrhosis, hepatocellular carcinoma, etc.).

Methods according to embodiments of the application further comprise administering to the subject in need thereof another immunogenic agent (such as another HBV antigen or other antigen) or another anti-HBV agent (such as a nucleoside analog or other anti-HBV agent) in combination with a composition of the application. For example, another anti-HBV agent or immunogenic agent can be a small molecule or antibody including, but not limited to, immune checkpoint inhibitors (e.g., anti-PD1, anti-TIM-3, etc.), toll-like receptor agonists (e.g., TLR7 agonists and/oror TLR8 agonists), RIG-1 agonists, IL-15 superagonists (Altor Bioscience), mutant IRF3 and IRF7 genetic adjuvants, STING agonists (Aduro), FLT3L genetic adjuvant, IL12 genetic adjuvant, IL-7-hyFc; CAR-T which bind HBV env (S-CAR cells); capsid assembly modulators; cccDNA inhibitors, HBV polymerase inhibitors (e.g., entecavir and tenofovir). The one or other anti-HBV active agents can be, for example, a small molecule, an antibody or antigen binding fragment thereof, a polypeptide, protein, or nucleic acid. The one or other anti-HBV agents can e.g., be chosen from among HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDM5 inhibitors.

### Methods of Delivery

Compositions and therapeutic combinations of the application can be administered to a subject by any method known in the art in view of the present disclosure, including, but not limited to, parenteral administration (e.g., intramuscular, subcutaneous, intravenous, or intradermal injection), oral administration, transdermal administration, and nasal administration.

Preferably, compositions and therapeutic combinations are administered parenterally (e.g., by intramuscular injection or intradermal injection) or transdermally.

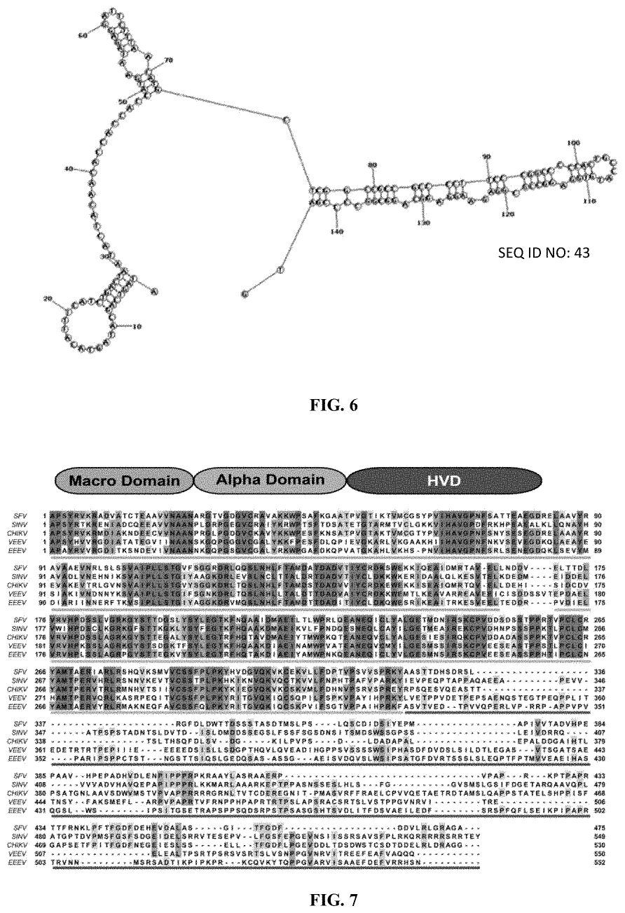

The molecules and/or compositions of the disclosure can be formulated using one or more liposomes, lipoplexes, and/or lipid nanoparticles. In one embodiment, pharmaceutical formulations of the molecules and/or compositions of the disclosure include liposomes (see, e.g., FIG. 5A and FIG. 5B). Liposomes are artificially-prepared vesicles which can primarily be composed of a lipid bilayer and can be used as a delivery vehicle for the administration of nutrients and pharmaceutical formulations. Liposomes can be of different sizes such as, but not limited to, a multilamellar vesicle (MLV) which can be hundreds of nanometers in diameter and can contain a series of concentric bilayers separated by narrow aqueous compartments, a small unicellular vesicle (SUV) which can be smaller than 50 nm in diameter, and a large unilamellar vesicle (LUV) which can be between 50 and 500 nm in diameter. Liposome design can include, but is not limited to, opsonins or ligands in order to improve the attachment of liposomes to unhealthy tissue or to activate events such as, but not limited to, endocytosis. Liposomes can contain a low or a high pH in order to improve the delivery of the pharmaceutical formulations.

The formation of liposomes can depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, the molecules and/or compositions of the disclosure can be formulated in a lipid vesicle which can have crosslinks between functionalized lipid bilayers. In some embodiments, the molecules and/or compositions of the disclosure can be formulated in a lipid-polycation complex. The formation of the lipid-polycation complex can be accomplished by methods known in the art. As a non-limiting example, the polycation can include a cationic peptide or a polypeptide such as, but not limited to, polylysine, polyornithine and/or polyarginine and the cationic peptides. In some embodiments, the nucleic acid molecules and/or compositions disclosed herein can be formulated in a lipid-polycation complex which can further include a neutral lipid such as, but not limited to, cholesterol or dioleoyl phosphatidylethanolamine (DOPE). The liposome formulation can be influenced by, but not limited to, the selection of the cationic lipid component, the degree of cationic lipid saturation, the nature of the PEGylation, ratio of all components and biophysical parameters such as size.

In some embodiments, the ratio of PEG in the lipid nanoparticle (LNP) formulations can be increased or decreased and/or the carbon chain length of the PEG lipid can be modified from C14 to C18 to alter the pharmacokinetics and/or biodistribution of the LNP formulations. As a non-limiting example, LNP formulations can contain 1-5% of the lipid molar ratio of PEG-c-DOMG as compared to the cationic lipid, DSPC and cholesterol. In another embodiment, the PEG-c-DOMG can be replaced with a PEG lipid such as, but not limited to, PEG-DSG (1,2-Distearoyl-sn-glycerol, methoxypolyethylene glycol) or PEG-DPG (1,2-Dipalmitoyl-sn-glycerol, methoxypolyethylene glycol). The cationic lipid can be selected from any lipid known in the art such as, but not limited to, DLin-MC3-DMA, DLin-DMA, C12-200, and DLin-KC2-DMA.

In some embodiments, LNP formulations described herein can comprise a polycationic composition. In some embodiments, the LNP formulations comprising a polycationic composition can be used for the delivery of the modified RNA described herein in vivo and/or ex vitro. In some embodiments, the LNP formulations described herein can additionally comprise a permeability enhancer molecule. The nanoparticle formulations can be a carbohydrate nanoparticle comprising a carbohydrate carrier and a modified nucleic acid molecule (e.g., mRNA). As a non-limiting example, the carbohydrate carrier can include, but is not limited to, an anhydride-modified phytoglycogen or glycogen-type material, phtoglycogen octenyl succinate, phytoglycogen beta-dextrin, and anhydride-modified phytoglycogen beta-dextrin.

Lipid nanoparticle formulations can be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and can be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it can be terminally located at the terminal end of the lipid chain. The internal ester linkage can replace any carbon in the lipid chain.

Additional disclosure on lipid compositions useful for delivering a nucleic acid molecule encoding one or more HBV antigens can be found from U.S. Provisional Patent Application No. 62/863,958 entitled “Lipid Nanoparticle or Liposome Delivery of Hepatitis B Virus (HBV) Vaccines,” filed on Jun. 20, 2019, the content of which is hereby incorporated by reference in its entirety.

The molecules and/or compositions of the disclosure can also be formulated as a nanoparticle using a combination of polymers, lipids, and/or other biodegradable agents, such as, but not limited to, calcium phosphate, polymers. Components can be combined in a core-shell, hybrid, and/or layer-by-layer architecture, to allow for fine-tuning of the nanoparticle so that delivery of the molecules and/or compositions of the disclosure can be enhanced.

Additional disclosure on compositions useful for delivering a nucleic acid molecule encoding one or more HBV antigens can be found from U.S. Provisional Patent Application No. 62/863,950 entitled “Carbohydrate Nanoparticle Delivery of Hepatitis B Virus (HBV) Vaccines,” filed on Jun. 20, 2019, the content of which is hereby incorporated by reference in its entirety.

Methods of delivery are not limited to the above described embodiments, and any means for intracellular delivery can be used.

### Adjuvants

In some embodiments of the application, a method of inducing an immune response against HBV further comprises administering an adjuvant. The terms “adjuvant” and “immune stimulant” are used interchangeably herein and are defined as one or more substances that cause stimulation of the immune system. In this context, an adjuvant is used to enhance an immune response to HBV antigens and antigenic HBV polypeptides of the application.

According to embodiments of the application, an adjuvant can be present in a therapeutic combination or composition of the application or administered in a separate composition. An adjuvant can be, e.g., a small molecule or an antibody. Examples of adjuvants suitable for use in the application include, but are not limited to, immune checkpoint inhibitors (e.g., anti-PD1, anti-TIM-3, etc.), toll-like receptor agonists (e.g., TLR7 and/or TLR8 agonists), RIG-1 agonists, IL-15 superagonists (Altor Bioscience), mutant IRF3 and IRF7 genetic adjuvants, STING agonists (Aduro), FLT3L genetic adjuvant, IL12 genetic adjuvant, and IL-7-hyFc. Examples of adjuvants can e.g., be chosen from among the following anti-HBV agents: HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDM5 inhibitors.

Compositions and therapeutic combinations of the application can also be administered in combination with at least one other anti-HBV agent. Examples of anti-HBV agents suitable for use with the application include, but are not limited to small molecules, antibodies, and/or CAR-T therapies which bind HBV env (S-CAR cells), capsid assembly modulators, TLR agonists (e.g., TLR7 and/or TLR8 agonists), cccDNA inhibitors, HBV polymerase inhibitors (e.g., entecavir and tenofovir), and/or immune checkpoint inhibitors, etc.

The at least one anti-HBV agent can, e.g., be chosen from among HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDM5 inhibitors. Such anti-HBV agents can be administered with the compositions and therapeutic combinations of the application simultaneously or sequentially.

### Methods of Prime/Boost Immunization

Embodiments of the application also contemplate administering an immunogenically effective amount of a composition or therapeutic combination to a subject, and subsequently administering another dose of an immunogenically effective amount of a composition or therapeutic combination to the same subject, in a so-called prime-boost regimen. Thus, in an embodiment, a composition or therapeutic combination of the application is a primer vaccine used for priming an immune response. In another embodiment, a composition or therapeutic combination of the application is a booster vaccine used for boosting an immune response. The priming and boosting vaccines of the application can be used in the methods of the application described herein. This general concept of a prime-boost regimen is well known to the skilled person in the vaccine field. Any of the compositions and therapeutic combinations of the application described herein can be used as priming and/or boosting vaccines for priming and/or boosting an immune response against HBV.

In some embodiments of the application, a composition or therapeutic combination of the application can be administered for priming immunization. The composition or therapeutic combination can be re-administered for boosting immunization. Further booster administrations of the composition or vaccine combination can optionally be added to the regimen, as needed. An adjuvant can be present in a composition of the application used for boosting immunization, present in a separate composition to be administered together with the composition or therapeutic combination of the application for the boosting immunization, or administered on its own as the boosting immunization. In those embodiments in which an adjuvant is included in the regimen, the adjuvant is preferably used for boosting immunization.

An illustrative and non-limiting example of a prime-boost regimen includes administering a single dose of an immunogenically effective amount of a composition or therapeutic combination of the application to a subject to prime the immune response; and subsequently administering another dose of an immunogenically effective amount of a composition or therapeutic combination of the application to boost the immune response, wherein the boosting immunization is first administered about two to six weeks, preferably four weeks after the priming immunization is initially administered. Optionally, about 10 to 14 weeks, preferably 12 weeks, after the priming immunization is initially administered, a further boosting immunization of the composition or therapeutic combination, or other adjuvant, is administered.

### Kits

Also provided herein is a kit comprising a self-replicating RNA molecule of the application. A kit can comprise a self-replicating RNA molecule encoding the first polynucleotide and a self-replicating RNA molecule encoding the second polynucleotide in one or more separate compositions, or a kit can comprise a self-replicating RNA molecule encoding the first polynucleotide and a self-replicating RNA molecule encoding the second polynucleotide in a single composition. A kit can further comprise one or more adjuvants or immune stimulants, and/or other anti-HBV agents.

The ability to induce or stimulate an anti-HBV immune response upon administration in an animal or human organism can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed. J Wiley & Sons Inc, National Institute of Health). Measurement of cellular immunity can be performed by measurement of cytokine profiles secreted by activated effector cells including those derived from CD4+ and CD8+ T-cells (e.g. quantification of IL-10 or IFN gamma-producing cells by ELISPOT), by determination of the activation status of immune effector cells (e.g. T cell proliferation assays by a classical [3H] thymidine uptake or flow cytometry-based assays), by assaying for antigen-specific T lymphocytes in a sensitized subject (e.g. peptide-specific lysis in a cytotoxicity assay, etc.).

The ability to stimulate a cellular and/or a humoral response can be determined by antibody binding and/or competition in binding (see for example Harlow, 1989, Antibodies, Cold Spring Harbor Press). For example, titers of antibodies produced in response to administration of a composition providing an immunogen can be measured by enzyme-linked immunosorbent assay (ELISA). The immune responses can also be measured by neutralizing antibody assay, where a neutralization of a virus is defined as the loss of infectivity through reaction/inhibition/neutralization of the virus with specific antibody. The immune response can further be measured by Antibody-Dependent Cellular Phagocytosis (ADCP) Assay.

### Embodiments

The invention provides also the following non-limiting embodiments.

Embodiment 1 is a self-replicating RNA molecule, comprising at least one of:
a) a first polynucleotide sequence encoding the truncated HBV core antigen consisting of an amino acid sequence that is at least 95% identical to SEQ ID NO: 2; and
b) a second polynucleotide sequence encoding the HBV polymerase antigen consisting of an amino acid sequence that is at least 90% identical to SEQ ID NO: 7, wherein the HBV polymerase antigen does not have reverse transcriptase activity and RNase H activity.

Embodiment 1a is the self-replicating RNA molecule of embodiment 1, wherein the self-replicating RNA molecule comprises a feature that enhances expression of the encoded truncated HBV core antigen or the encoded HBV polymerase antigen when the self-replicating RNA molecule is administered to a cell.

Embodiment 1b is the self-replicating RNA molecule of embodiment 1a, comprising:
a) one or more nonstructural genes nsP1, nsP2, nsP3 and nsP4;
b) at least one of a DLP motif and a modified 5′-UTR;
c) a subgenomic promoter; and
d) at least one of
   i. a first polynucleotide sequence encoding a truncated HBV core antigen consisting of an amino acid sequence that is at least 95% identical to SEQ ID NO: 2; and
   ii. a second polynucleotide sequence encoding the HBV polymerase antigen consisting of an amino acid sequence that is at least 90% identical to SEQ ID NO: 7, wherein the HBV polymerase antigen does not have reverse transcriptase activity and RNase H activity;
operably linked to the subgenomic promoter.

Embodiment 2 is the self-replicating RNA molecule of any one of embodiments 1-1b, comprising the first polynucleotide sequence encoding a truncated HBV core antigen consisting of an amino acid sequence that is at least 95% identical to SEQ ID NO: 2.

Embodiment 3 is the self-replicating RNA molecule of embodiment 2, comprising the second polynucleotide encoding the HBV polymerase antigen consisting of an amino acid sequence that is at least 90% identical to SEQ ID NO: 7, wherein the HBV polymerase antigen does not have reverse transcriptase activity and RNase H activity.

Embodiment 4 is the self-replicating RNA molecule of embodiment 3, comprising:
a) a first polynucleotide sequence encoding a truncated HBV core antigen consisting of the amino acid sequence of SEQ ID NO: 2; and
b) a second polynucleotide sequence encoding the HBV polymerase antigen comprising the amino acid sequence of SEQ ID NO: 7, wherein the HBV polymerase antigen does not have reverse transcriptase activity and RNase H activity.

Embodiment 5 the self-replicating RNA molecule of any one of embodiments 1-4, wherein the first polynucleotide further comprises a polynucleotide sequence encoding a signal sequence operably linked to the N-terminus of the truncated HBV core antigen.

Embodiment 5a is the self-replicating RNA molecule of any one of embodiments 1-5, wherein the second polynucleotide further comprises further comprises a polynucleotide sequence encoding a signal sequence operably linked to the N-terminus of the HBV polymerase antigen.

Embodiment 5b is the self-replicating RNA molecule of embodiment 5 or 5a, wherein the signal sequence independently comprises the amino acid sequence of SEQ ID NO: 9 or SEQ ID NO: 15.

Embodiment 5c is the self-replicating RNA molecule of embodiment 5 or 5a, wherein the signal sequence is independently encoded by the polynucleotide sequence of SEQ ID NO: 8 or SEQ ID NO: 14.

Embodiment 6 is the self-replicating RNA molecule of any one of embodiments 1-5c, wherein the HBV polymerase antigen comprises an amino acid sequence that is at least 98%, such as at least 98%, 98.5%, 99%, 99.1%, 99.2%, 99.3%, 99.4%, 99.5%, 99.6%, 99.7%, 99.8%, 99.9%, or 100%, identical to SEQ ID NO: 7.

Embodiment 6a is the self-replicating RNA molecule of embodiment 6, wherein the HBV polymerase antigen comprises the amino acid sequence of SEQ ID NO: 7.

Embodiment 6b is the self-replicating RNA molecule of any one of embodiments 1 to 6a, wherein and the truncated HBV core antigen consists of the amino acid sequence that is at least 98%, such as at least 98%, 98.5%, 99%, 99.1%, 99.2%, 99.3%, 99.4%, 99.5%, 99.6%, 99.7%, 99.8%, 99.9%, or 100%, identical to SEQ ID NO: 2.

Embodiment 6c is the self-replicating RNA molecule of embodiment 6b, wherein the truncated HBV antigen consists of the amino acid sequence of SEQ ID NO: 2 or SEQ ID NO: 4.

Embodiment 7 is the self-replicating RNA molecule of any one of embodiments 1-6c, wherein the first polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 1 or SEQ ID NO: 3.

Embodiment 7a is the self-replicating RNA molecule of embodiment 7, wherein the first polynucleotide sequence comprises a polynucleotide sequence having at least 98%, such as at least 98%, 98.5%, 99%, 99.1%, 99.2%, 99.3%, 99.4%, 99.5%, 99.6%, 99.7%, 99.8%, 99.9%, or 100%, sequence identity to SEQ ID NO: 1 or SEQ ID NO: 3.

Embodiment 8 is the self-replicating RNA molecule of embodiment 7a, wherein the first polynucleotide sequence comprises the polynucleotide sequence of SEQ ID NO: 1 or SEQ ID NO: 3.

Embodiment 9 the self-replicating RNA molecule of any one of embodiments 1 to 8, wherein the second polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 5 or SEQ ID NO: 6.

Embodiment 9a the self-replicating RNA molecule of embodiment 9, wherein the second polynucleotide sequence comprises a polynucleotide sequence having at least 98%, such as at least 98%, 98.5%, 99%, 99.1%, 99.2%, 99.3%, 99.4%, 99.5%, 99.6%, 99.7%, 99.8%, 99.9%, or 100%, sequence identity to SEQ ID NO: 5 or SEQ ID NO: 6.

Embodiment 10 is the self-replicating RNA molecule of embodiment 9a, wherein the second polynucleotide sequence comprises the polynucleotide sequence of SEQ ID NO: 5 or SEQ ID NO: 6.

Embodiment 11 is the self-replicating RNA molecule of any one of embodiments 1 to 10, encoding a fusion protein comprising the truncated HBV core antigen operably linked to the HBV polymerase antigen.

Embodiment 12 is the self-replicating RNA molecule of embodiment 11, wherein the fusion protein comprises the truncated HBV core antigen operably linked to the HBV polymerase antigen via a linker.

Embodiment 13 is the self-replicating RNA molecule of embodiment 12, wherein the linker comprises the amino acid sequence of (AlaGly)n, and n is an integer of 2 to 5.

Embodiment 13a is the self-replicating RNA molecule of embodiment 13, wherein the linker is encoded by a polynucleotide sequence at least 90% identical to SEQ ID NO: 11, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 11.

Embodiment 13b is the self-replicating RNA molecule of embodiment 13a, wherein the linker is encoded by a polynucleotide sequence comprising SEQ ID NO: 11.

Embodiment 14 is the self-replicating RNA of any one of embodiments 13-13b, wherein the fusion protein comprises the amino acid sequence of SEQ ID NO: 16.

Embodiment 15 is the self-replicating RNA molecule of any one of embodiments 1-14, wherein the self-replicating RNA is an alphavirus-derived RNA replicon.

Embodiment 15a is the self-replicating RNA molecule of embodiment 15, wherein the self-replicating RNA comprises the DLP motif.

Embodiment 15b is the self-replicating RNA molecule of embodiment 15a, wherein the DLP motif is derived from a capsid gene of a virus species belonging to the Togaviridae family.

Embodiment 15c is the self-replicating RNA molecule of embodiment 15a or 15b, wherein the self-replicating RNA molecule further comprises a coding sequence for an autoprotease peptide operably linked downstream of the DLP motif and upstream of the first or second polynucleotide encoding the HBV protein.

Embodiment 15d is the self-replicating RNA molecule of embodiment 15c, wherein the autoprotease peptide is selected from the group consisting of porcine teschovirus-1 2A (P2A), a foot-and-mouth disease virus (FMDV) 2A (F2A), an Equine Rhinitis A Virus (FRAN) 2A (E2A), a Thosea. asigna virus 2A (T2A), a cytoplasmic polyhedrosis virus 2A (BmCPV2A), a Flacherie Virus 2A (BmIFV2A), and a combination thereof.

Embodiment 15e is the self-replicating RNA molecule of embodiment 15a, wherein the DLP motif and other genetic elements of the RNA replicon are described in US Patent Application Publication US2018/0171340 and the International Patent Application Publication WO2018106615, which are incorporated herein by reference.

Embodiment 16 is the self-replicating RNA molecule of any one of embodiments 1 to 15e, wherein the RNA replicon comprises alphavirus non-structural proteins nsP1, nsP2, nsP3 and nsP4.

Embodiment 16a is the self-replicating RNA molecule of embodiment 16, wherein the RNA replicon does not encode a functional alphavirus structural protein.

Embodiment 16b is the self-replicating RNA molecule of embodiment 16, wherein the RNA replicon encodes one or more functional alphavirus structural proteins.

Embodiment 16c is the self-replicating RNA molecule of embodiment 16, comprising the modified 5′ untranslated region (5′-UTR).

Embodiment 16d is the self-replicating RNA molecule of embodiment 16c, wherein the modified 5′-UTR comprises one or more nucleotide substitutions at position 1, 2, 4, or a combination thereof.

Embodiment 16e is the self-replicating RNA molecule of embodiment 16d, wherein the modified 5′-UTR comprises a nucleotide substitution at position 2, preferably, the modified 5′-UTR has a U->G substitution at position 2.

Embodiment 16f is the self-replicating RNA molecule of embodiment 15c, wherein the modified 5′-UTR and other genetic elements of the RNA replicon are described in US Patent Application Publication US2018/0104359 and the International Patent Application Publication WO2018075235, the content of each of which is incorporated herein by reference in its entirety.

Embodiment 16g is the self-replicating RNA molecule of embodiment 1, further comprising a nucleic acid molecule having a nucleotide sequence exhibiting at least 90%, at least 95%, at least 96%, at least 97%, at least 98%, at least 99%, or 100% sequence identity to the nucleic acid sequence of any one of SEQ ID NOs: 25 to 42, and a U->G substitution at position 2 of the 5′-UTR, and wherein the modified alphavirus genome or replicon RNA is devoid of at least a portion of the sequence encoding viral structural proteins.

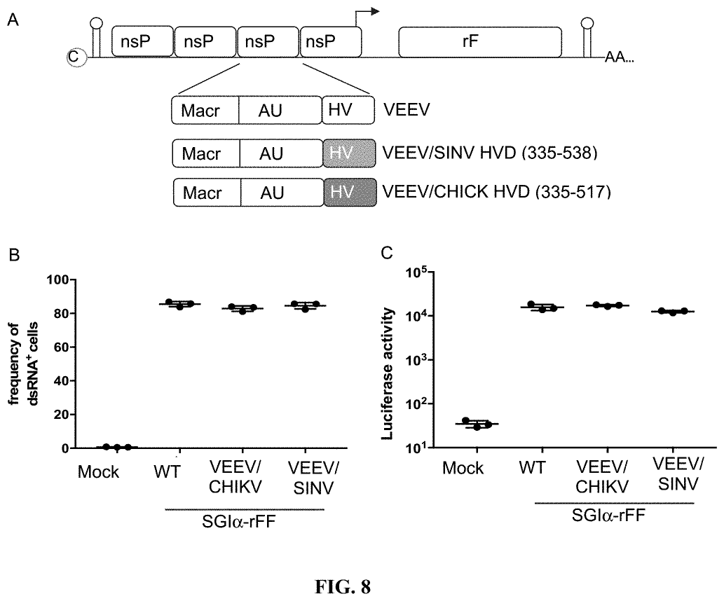

Embodiment 17 is a nucleic acid molecule, comprising (i) a first nucleic acid sequence encoding one or more RNA stem-loops of a viral capsid enhancer (FIG. 6) or a variant thereof; and (ii) a second nucleic acid sequence operably linked to the first nucleic acid sequence, wherein the second nucleic acid sequence encoding a truncated HBV core antigen consisting of an amino acid sequence that is at least 95% identical to SEQ ID NO: 2; and a HBV polymerase antigen consisting of an amino acid sequence that is at least 90% identical to SEQ ID NO: 7, wherein the HBV polymerase antigen does not have reverse transcriptase activity and RNase H activity.

Embodiment 17a is the nucleic acid molecule of embodiment 17, further comprising a coding sequence for an autoprotease peptide operably linked upstream to the second nucleic acid sequence, preferably the coding sequence for the autoprotease peptide is operably linked downstream to the first nucleic acid sequence and upstream to the second nucleic acid sequence.

Embodiment 17b is the nucleic acid molecule of embodiment 17a, wherein the autoprotease peptide comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof.

Embodiment 17c is the nucleic acid molecule of any one of embodiments 17-17b, wherein the viral capsid enhancer is derived from a capsid gene of a virus species belonging to the Togaviridae family. In some embodiments, the alphavirus species is Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), Salmonid alphavirus (SAV), or Buggy Creek virus, preferably the viral capsid enhancer comprises a downstream loop (DLP) motif of the virus species.

Embodiment 17d is the nucleic acid molecule of any one of embodiments 17-17c, wherein the viral capsid enhancer comprises a nucleic acid sequence exhibiting at least 80%, 85%, 90%, 95% or 100% sequence identity to at least one of SEQ ID NOs: 43-50.

Embodiment 17e is the nucleic acid molecule of any one of embodiments 17-17d, further comprising a third nucleic acid sequence encoding one or more RNA stem-loops of a second viral capsid enhancer or a variant thereof and a fourth nucleic acid sequence operably linked to the third nucleic acid sequence, wherein the fourth nucleic acid sequence comprises a coding sequence for a second gene of interest (GOI).

Embodiment 17f is the nucleic acid molecule of embodiment 17e, further comprising a coding sequence for a second autoprotease peptide operably linked downstream to the third nucleic acid sequence and upstream to the fourth nucleic acid sequence.

Embodiment 17g is the nucleic acid molecule of any one of embodiments 17 to 17f, wherein the self-replicating RNA molecule contains New World alphavirus nonstructural proteins nsP1, nsP2, and nsP4; and an alphavirus nsP3 protein macro domain, central domain, and hypervariable domain, wherein the hypervariable domain is derived from an Old World alphavirus nsP3 hypervariable domain, or a chimeric nsP3 hypervariable domain derived from a portion of a New World alphavirus nsP3 hypervariable domain and another portion from an Old World alphavirus nsP3 hypervariable domain.

Embodiment 17h is the nucleic acid molecule of embodiment 17g, wherein the alphavirus nsP3 macro domain and the alphavirus nsP3 central domain are from a New World alphavirus.

Embodiment 17i is the nucleic acid molecule of embodiment 17g, wherein the alphavirus nsP3 macro domain and the alphavirus nsP3 central domain are from an Old World alphavirus.

Embodiment 17j is the nucleic acid molecule of any one of embodiment 17g to 17i, wherein the portion derived from the Old World alphavirus nsP3 hypervariable domain comprises a motif selected from the group consisting of FGDF and FGSF.

Embodiment 17k is the nucleic acid molecule of embodiment 17j, wherein the Old World alphavirus nsP3 hypervariable domain comprises a repeat selected from the group consisting of: an FGDF/FGDF repeat, an FGSF/FGSF repeat, an FGDF/FGSF repeat, and an FGSF/FGDF repeat, preferably the repeat sequences are separated by at least 10 and not more than 25 amino acids.

Embodiment 17l is the nucleic acid molecule of embodiment 17k, wherein the repeat sequences are separated by an amino acid sequence derived from the group consisting of: SEQ ID NO: 56: NEGEIESLSSELLT and SEQ ID NO: 57: SDGEIDELSRRVTTESEPVL and SEQ ID NO: 58: DEHEVDALASGIT.

Embodiment 17m is the nucleic acid molecule of any one of embodiment 17g to 171, wherein the portion derived from the Old World alphavirus hypervariable domain can have any of amino acids 479-482 or 497-500 or 479-500 or 335-517 of CHIKV nsP3 HVD; or any of amino acids 451-454 or 468-471 or 451-471 of SFV nsP3 HVD; or amino acids 490-493 or 513-516 or 490-516 or 335-538 of SINV nsP3 HVD.

Embodiment 17m is the nucleic acid molecule of any one of embodiment 17g to 17m, wherein the New World alphavirus can be VEEV and the portion derived from the New World alphavirus hypervariable domain does not comprise amino acids 478-518 of the VEEV nsP3 hypervariable domain; or does not comprise amino acids 478-545 of the VEEV nsP3 hypervariable domain; or does not comprise amino acids 335-518 of the VEEV nsP3 hypervariable domain.

Embodiment 17n is the nucleic acid molecule of any one of embodiment 17g to 17m, wherein the New World alphavirus can be EEEV and the portion derived from the New World alphavirus hypervariable domain does not comprise amino acids 531-547 of the EEEV hypervariable domain, or the New World alphavirus can be WEEV, and the portion derived from the New World alphavirus hypervariable domain does not comprise amino acids 504-520 of the WEEV hypervariable domain.

Embodiment 17o is the nucleic acid molecule of any one of embodiment 17g to 17n, wherein the New World alphavirus is EEEV, the nsP2/nsP3 sequence can be (SEQ ID NO: 64) QHEAGR/APAY, and with the penultimate G preserved, preferably the sequence at the nsP3/nsP4 junction can be (SEQ ID NO: 65) RYEAGA/YIFS, and the penultimate glycine can be optionally preserved while the remaining nsP3 amino acids varied as described herein; these sequences can also be preceded by a read-through stop codon (TGA).

Embodiment 17p is the nucleic acid molecule of any one of embodiment 17g to 17n, wherein the New World alphavirus is WEEV, and the nsP2/nsP3 junction can be (SEQ ID NO: 66) RYEAGR/APAY, and the penultimate G preserved while the remaining amino acids in the nsP2/nsP3 junction are varied as described herein, preferably the nsP3/nsP4 junction of WEEV, the sequence can be (SEQ ID NO: 67) RYEAGA/YIFS, with the penultimate glycine preserved and the remaining nsP3 amino acids varied as described herein; these sequences can also be preceded by a read-through stop codon (TGA).

Embodiment 17q is the nucleic acid molecule of embodiment 17o or 17p, wherein the sequences of SEQ ID NOs: 62-67 can also contain one or two or three substitutions on the N-terminal and/or C-terminal sides.

Embodiment 18 is a composition comprising the self-replicating RNA of any one of embodiments 1-17p and a pharmaceutically acceptable carrier.

Embodiment 19 is the composition of embodiment 18 wherein the self-replicating RNA molecule is encapsulated in, bound to or adsorbed on a liposome, a lipoplex, a lipid nanoparticle, or combinations thereof.

Embodiment 20 is the composition of embodiment 19, wherein the self-replicating RNA molecule is encapsulated in a lipid nanoparticle.

Embodiment 21 is a kit comprising the self-replicating RNA molecules of any one of embodiments 1 to 17p or the composition of any of embodiments 19-20, and instructions for using the therapeutic combination in treating a hepatitis B virus (HBV) infection in a subject in need thereof.

Embodiment 22 is a method of treating a hepatitis B virus (HBV) infection in a subject in need thereof, comprising administering to the subject self-replicating RNA molecule of any one of embodiments 1 to 17p or the composition of any one of embodiments 19-20.

Embodiment 22a is the method of embodiment 22, wherein the treatment induces an immune response against a hepatitis B virus in a subject in need thereof, preferably the subject has chronic HBV infection.

Embodiment 22b is the method of embodiment 22 or 22a, wherein the subject has chronic HBV infection.

Embodiment 22c is the method of any one of embodiments 22 to 22b, wherein the subject is in need of a treatment of an HBV-induced disease selected from the group consisting of advanced fibrosis, cirrhosis and hepatocellular carcinoma (HCC).

Embodiment 22d is the method of any one of embodiments 22-22c, wherein the composition is administered by injection through the skin, e.g., intramuscular or intradermal injection, preferably intramuscular injection.

### EXAMPLES

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### Example 1

### HBV Core Plasmid & HBV Pol Plasmid

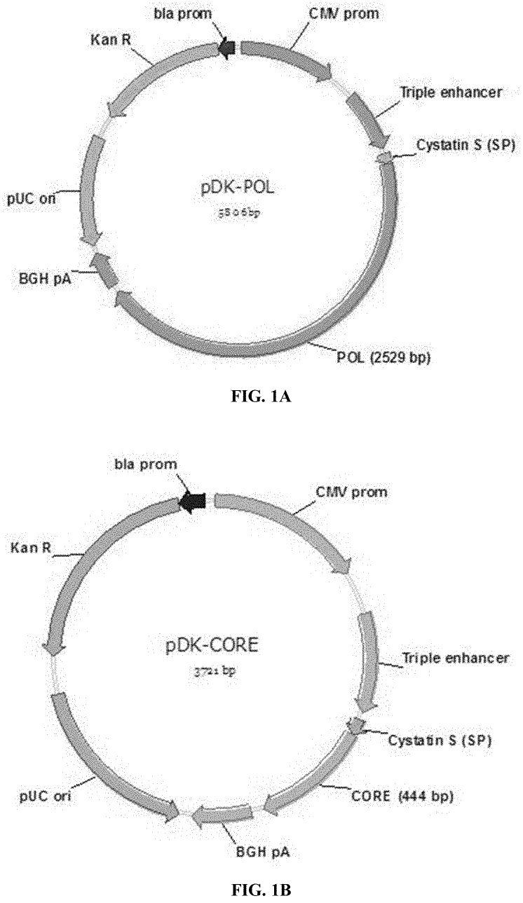

A schematic representation of the pDK-pol and pDK-core vectors is shown in FIGS. 1A and 1B, respectively. An HBV core or pol antigen optimized expression cassette containing a CMV promoter (SEQ ID NO: 18), a splicing enhancer (triple composite sequence) (SEQ ID NO: 10), Cystatin S precursor signal peptide SPCS (NP_0018901.1) (SEQ ID NO: 9), and pol (SEQ ID NO: 5) or core (SEQ ID NO: 2) gene was introduced into a pDK plasmid backbone, using standard molecular biology techniques.

The plasmids were tested in vitro for core and pol antigen expression by Western blot analysis using core and pol specific antibodies, and were shown to provide consistent expression profile for cellular and secreted core and pol antigens (data not shown).

### Example 2

### Generation of Adenoviral Vectors Expressing a Fusion of Truncated HBV Core Antigen with HBV Pol Antigen

The creation of an adenovirus vector has been designed as a fusion protein expressed from a single open reading frame. Additional configurations for the expression of the two proteins, e.g. using two separate expression cassettes, or using a 2A-like sequence to separate the two sequences, can also be envisaged.

### Design of Expression Cassettes for Adenoviral Vectors

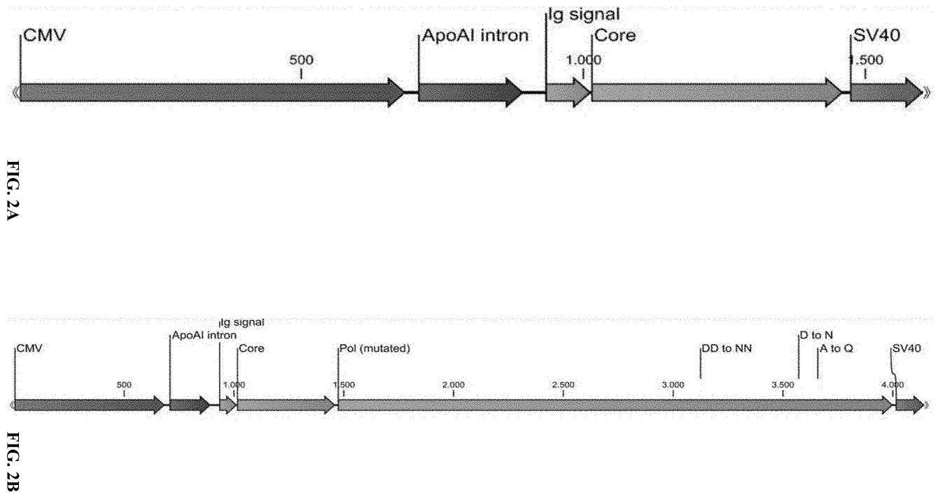

The expression cassettes (diagrammed in FIG. 2A and FIG. 2B) are comprised of the CMV promoter (SEQ ID NO: 19), an intron (SEQ ID NO:12) (a fragment derived from the human ApoAI gene-GenBank accession X01038 base pairs 295-523, harboring the ApoAI second intron), followed by the optimized coding sequence—either core alone or the core and polymerase fusion protein preceded by a human immunoglobulin secretion signal coding sequence (SEQ ID NO: 14), and followed by the SV40 polyadenylation signal (SEQ ID NO: 13).

A secretion signal was included because of past experience showing improvement in the manufacturability of some adenoviral vectors harboring secreted transgenes, without influencing the elicited T-cell response (mouse experiments).

The last two residues of the Core protein (VV) and the first two residues of the Polymerase protein (MP) if fused results in a junction sequence (VVMP) that is present on the human dopamine receptor protein (D3 isoform), along with flanking homologies.

The interjection of an AGAG linker between the core and the polymerase sequences eliminates this homology and returned no further hits in a Blast of the human proteome.

### Example 3

### In Vivo Immunogenicity Study of DNA Vaccine in Mice

An immunotherapeutic DNA vaccine containing DNA plasmids encoding an HBV core antigen or HBV polymerase antigen was tested in mice. The purpose of the study was designed to detect T-cell responses induced by the vaccine after intramuscular delivery via electroporation into BALB/c mice. Initial immunogenicity studies focused on determining the cellular immune responses that would be elicited by the introduced HBV antigens.

In particular, the plasmids tested included a pDK-Pol plasmid and pDK-Core plasmid, as shown in FIGS. 1A and 1B, respectively, and as described above in Example 1. The pDK-Pol plasmid encoded a polymerase antigen having the amino acid sequence of SEQ ID NO: 7, and the pDK-Core plasmid encoding a Core antigen having the amino acid sequence of SEQ ID NO: 2. First, T-cell responses induced by each plasmid individually were tested. The DNA plasmid (pDNA) vaccine was intramuscularly delivered via electroporation to Balb/c mice using a commercially available TriGrid™ delivery system-intramuscular (TDS-IM) adapted for application in the mouse model in cranialis tibialis. See International Patent Application Publication WO2017172838, and U.S. Patent Application No. 62/607,430, entitled “Method and Apparatus for the Delivery of Hepatitis B Virus (HBV) Vaccines,” filed on Dec. 19, 2017 for additional description on methods and devices for intramuscular delivery of DNA to mice by electroporation, the disclosures of which are hereby incorporated by reference in their entireties. In particular, the TDS-IM array of a TDS-IM v1.0 device having an electrode array with a 2.5 mm spacing between the electrodes and an electrode diameter of 0.030 inch was inserted percutaneously into the selected muscle, with a conductive length of 3.2 mm and an effective penetration depth of 3.2 mm, and with the major axis of the diamond configuration of the electrodes oriented in parallel with the muscle fibers. Following electrode insertion, the injection was initiated to distribute DNA (e.g., 0.020 ml) in the muscle. Following completion of the IM injection, a 250 V/cm electrical field (applied voltage of 59.4-65.6 V, applied current limits of less than 4 A, 0.16 A/sec) was locally applied for a total duration of about 400 ms at a 10% duty cycle (i.e., voltage is actively applied for a total of about 40 ms of the about 400 ms duration) with 6 total pulses. Once the electroporation procedure was completed, the TriGrid™ array was removed and the animals were recovered. High-dose (20 μg) administration to BALB/c mice was performed as summarized in Table 1. Six mice were administered plasmid DNA encoding the HBV core antigen (pDK-core; Group 1), six mice were administered plasmid DNA encoding the HBV pol antigen (pDK-pol; Group 2), and two mice received empty vector as the negative control. Animals received two DNA immunizations two weeks apart and splenocytes were collected one week after the last immunization.

TABLE 1
Mouse immunization experimental design of the pilot study.

| | | | Unilateral | | | | Endpoint |
|---|---|---|---|---|---|---|---|
| | | | Admin Site | | | | (spleen |
| | | | (alternate | | | Admin | harvest) |
| Group | N | pDNA | sides) | Dose | Vol | Days | Day |
| 1 | 6 | Core | CT + EP | 20 μg | 20 μL | 0, 14 | 21 |
| 2 | 6 | Pol | CT + EP | 20 μg | 20 μL | 0, 14 | 21 |
| 3 | 2 | Empty | CT + EP | 20 μg | 20 μL | 0, 14 | 21 |
| | | Vector | | | | | |
| | | (neg control) | | | | | |

CT, cranialis tibialis muscle; EP, electroporation.

Antigen-specific responses were analyzed and quantified by IFN-γ enzyme-linked immunospot (ELISPOT). In this assay, isolated splenocytes of immunized animals were incubated overnight with peptide pools covering the Core protein, the Pol protein, or the small peptide leader and junction sequence (2 μg/ml of each peptide). These pools consisted of 15 mer peptides that overlap by 11 residues matching the Genotypes BCD consensus sequence of the Core and Pol vaccine vectors. The large 94 kDan HBV Pol protein was split in the middle into two peptide pools. Antigen-specific T cells were stimulated with the homologous peptide pools and IFN-γ-positive T cells were assessed using the ELISPOT assay. IFN-γ release by a single antigen-specific T cell was visualized by appropriate antibodies and subsequent chromogenic detection as a colored spot on the microplate referred to as spot-forming cell (SFC).

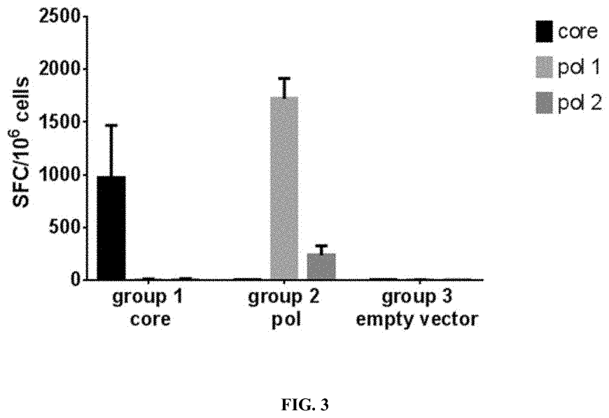

Substantial T-cell responses against HBV Core were achieved in mice immunized with the DNA vaccine plasmid pDK-Core (Group 1) reaching 1,000 SFCs per 10⁶ cells (FIG. 3). Pol T-cell responses towards the Pol 1 peptide pool were strong (˜1,000 SFCs per 10⁶ cells). The weak Pol-2-directed anti-Pol cellular responses were likely due to the limited MHC diversity in mice, a phenomenon called T-cell immunodominance defined as unequal recognition of different epitopes from one antigen. A confirmatory study was performed confirming the results obtained in this study (data not shown).

The above results demonstrate that vaccination with a DNA plasmid vaccine encoding HBV antigens induces cellular immune responses against the administered HBV antigens in mice. Similar results were also obtained with non-human primates (data not shown).

### Example 4

### Immunogenicity of VEEV-Based Replicon

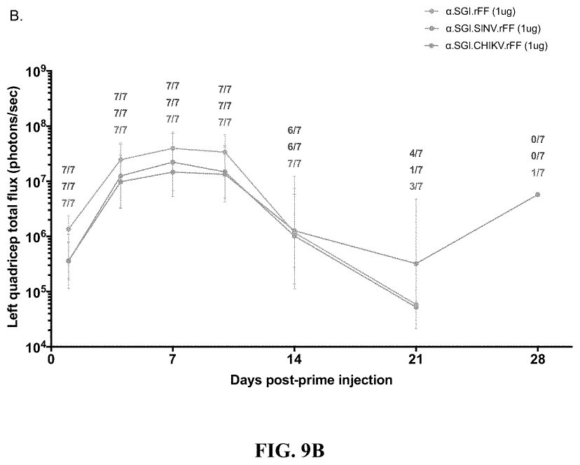

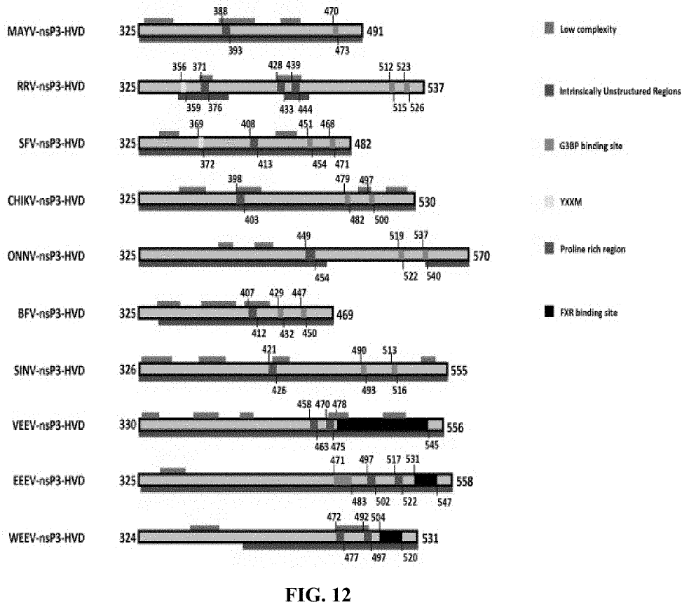

A VEEV-based alphavirus replicon encoding a mutant nsP3 was constructed by replacing the nucleotide sequence encoding amino acids 335-518 of VEEV nsP3 with a nucleotide sequence encoding amino acids 335-517 of the Chikungunya nsP3 to create a VEEV based replicon expressing a VEEV/CHIKV nsP3 chimera. This replacement removed the first motif of a repeat sequence from VEEV, and replaced it with a FGDF/FGDF repeat sequence from the CHIKV genome (at amino acids 479-482 and 497-500). In a parallel experiment amino acids 335-538 of VEEV nsP3 (HVD region) were replaced with amino acids 335-538 of Sindbis virus nsP3 amino acids (HVD region) to generate a replicon encoding a VEEV/SINV nsP3 chimera (FIGS. 8 and 12). This replacement removed a repeat sequence from VEEV and replaced it with a FGSF/FGSF repeat sequence from SINV. Replicons containing the VEEV/CHIKV or VEEV/SINV chimeric nsP3 and expressing a red firefly luciferase (rFF) reporter from the subgenomic RNA were delivered into BHK-21 cells in triplicate by electroporation. Following electroporation a portion of the cells were plated into one well of a 6 well plate and one well of a 96 well plate and allowed to recover for 20 hours. Electroporated cells were stained for the presence of dsRNA and analyzed by flow cytometry to determine the frequency of dsRNA positive cells as a measure of replicon amplification. We found that replicons containing mutant nsP3 replicated to the same levels as a replicon containing a WT nsP3 (FIG. 8B). When analyzed for luciferase activity, no difference was seen between replicons containing WT or the indicated mutant forms of nsP3 (FIG. 8C).

### Example 5

### Expression of Heterologous Protein from Replicon

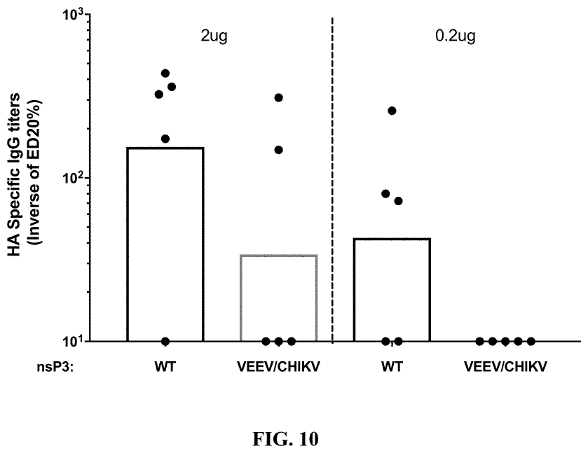

This example examined in vivo expression of recombinant firefly luciferase (rFF) from replicons (Example 4) encoding the mutant nsP3 as described in FIG. 8A. One or ten micrograms of replicon RNA in saline was delivered intramuscularly (IM) into the quadricep muscle of BALB/c mice. At indicated time points luciferase activity was monitored in vivo using a commercially available in vivo imaging system and reported as total flux (FIGS. 9A and 9B). The data show that replicons expressing mutant forms of nsP3 exhibited similar levels of luciferase activity in vivo compared to a replicon containing wild type nsP3 from VEEV.

### Example 6

### Immunogenicity

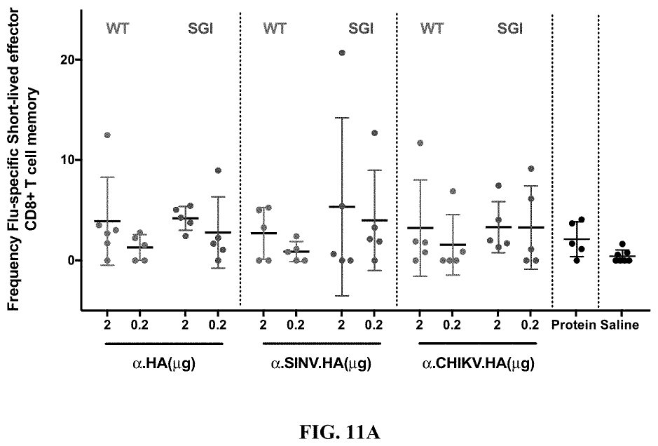

This example examined the immunogenicity of a VEEV-based replicon encoding a VEEV/CHIKV chimeric form of nsP3 (from Example 4) versus the immunogenicity of a replicon with a wild type (wt) VEEV nsP3. Each replicon encoded and expressed HA from the H5N1 strain of influenza as the heterologous protein. 2.0 ug or 0.2 ug of RNA in saline was delivered intra-muscularly to the quadricep muscle of BALB/c mice at day 0 and boosted with the same replicon RNA and dose at day 28. Two weeks post boost (day 42 post prime) spleens and serum were collected. Serum was analyzed for HA specific antibodies by ELISA (FIG. 10). The data show that replicons encoding a VEEV/CHIKV nsP3 chimera significantly reduced HA specific IgG titers compared to a replicon with wild type nsP3.

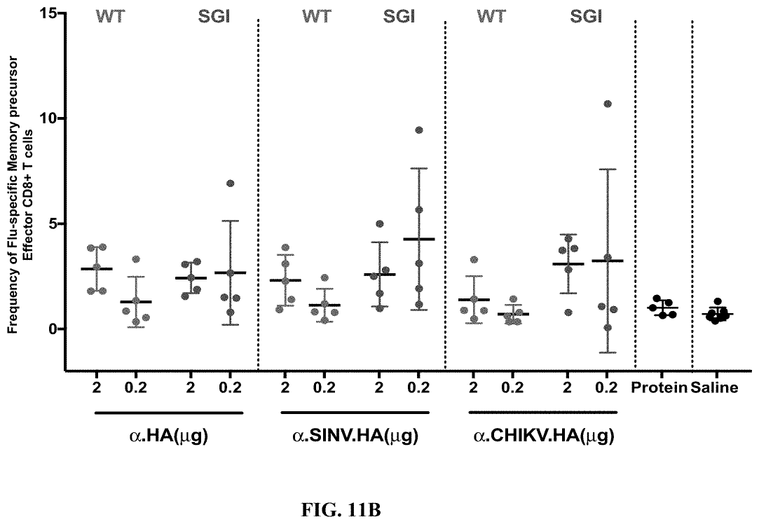

In contrast, analysis of the short-lived effector and memory precursor effector CD8+ T cells showed no difference in the frequency of HA specific cells between the different replicons tested (FIG. 9B and C). FIG. 11A shows similar frequency of HA specific short-lived effector CD8+ T cells between the wild type, VEEV/SINV nsP3, and VEEV/CHIKV nsP3 RNA replicons. FIG. 11B shows a similar result for memory effector CD8+ T cells.

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

1-22. (canceled)

23. A self-replicating RNA molecule comprising:
a non-naturally occurring polynucleotide sequence encoding a Hepatitis B virus (HBV) polymerase antigen consisting of an amino acid sequence that is at least 90% identical to SEQ ID NO: 7, wherein the HBV polymerase antigen does not have reverse transcriptase activity and RNase H activity and is capable of inducing a T cell response against at least HBV genotypes B, C and D;
wherein the self-replicating RNA molecule comprises a feature that enhances expression of the encoded HBV polymerase antigen upon delivery to a cell.

24. The self-replicating RNA molecule of claim 23, further comprising a non-naturally occurring polynucleotide sequence encoding a truncated HBV core antigen consisting of an amino acid sequence that is at least 95% identical to SEQ ID NO: 2.

25. A self-replicating RNA molecule comprising:
a) one or more nonstructural genes nsP1, nsP2, nsP3 and nsP4;
b) at least one of a DLP motif and a modified 5′-UTR;
c) a subgenomic promoter; and
d) a non-naturally occurring polynucleotide sequence encoding a Hepatitis B virus (HBV) polymerase antigen consisting of an amino acid sequence that is at least 90% identical to SEQ ID NO: 7, wherein the HBV polymerase antigen does not have reverse transcriptase activity and RNase H activity and is capable of inducing a T cell response against at least HBV genotypes B, C and D; wherein the subgenomic promoter is operably linked to the non-naturally occurring polynucleotide sequence encoding the HBV polymerase antigen.

26. The self-replicating RNA molecule of claim 25, further comprising a non-naturally occurring polynucleotide sequence encoding a truncated HBV core antigen consisting of an amino acid sequence that is at least 95% identical to SEQ ID NO: 2.

27. The self-replicating RNA molecule of claim 26, comprising the DLP motif, and a coding sequence for an autoprotease peptide operably linked downstream of the DLP motif and upstream to the first or second non-naturally occurring nucleic acid molecule.

28. The self-replicating RNA molecule of claim 27, wherein the autoprotease peptide is selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof.

29. The self-replicating RNA molecule of claim 25, comprising the modified 5′-UTR, wherein the modified 5′-UTR comprises one or more nucleotide substitutions at position 1, 2, 4, or a combination thereof.

30. The self-replicating RNA molecule of claim 23, comprising nonstructural genes nsP1, nsP2, nsP3 and nsP4, wherein the self-replicating RNA molecule does not encode a functional viral structural protein.

31. The self-replicating RNA molecule of claim 23, comprising nonstructural genes nsP1, nsP2, nsP3 and nsP4, wherein the self-replicating RNA molecule encodes one or more functional viral structural proteins.

32. The self-replicating RNA molecule of claim 30, wherein the self-replicating RNA molecule contains genes of New World alphavirus nonstructural proteins nsP1, nsP2, and nsP4; and encodes an alphavirus nsP3 protein macro domain, central domain, and hypervariable domain, wherein the hypervariable domain is derived from an Old World alphavirus nsP3 hypervariable domain, or a chimeric nsP3 hypervariable domain derived from a portion of a New World alphavirus nsP3 hypervariable domain and another portion from an Old World alphavirus nsP3 hypervariable domain.

33. The self-replicating RNA molecule of claim 23, wherein the HBV polymerase antigen consists of an amino acid sequence that is at least 98% identical to SEQ ID NO: 7.

34. The self-replicating RNA molecule of claim 23, further comprising a polynucleotide sequence encoding a signal sequence operably linked to the N-terminus of the HBV polymerase antigen.

35. The self-replicating RNA molecule of claim 24, wherein:
a) the truncated HBV core antigen consists of the amino acid sequence of SEQ ID NO: 2 or SEQ ID NO: 4; and
b) the HBV polymerase antigen comprises the amino acid sequence of SEQ ID NO: 7.

36. The self-replicating RNA molecule of claim 35, wherein the non-naturally occurring polynucleotide sequence encoding the core antigen comprises the polynucleotide sequence of SEQ ID NO: 1 or SEQ ID NO: 3, and the non-naturally occurring polynucleotide sequence encoding the polymerase antigen comprises the polynucleotide sequence of SEQ ID NO: 5 or SEQ ID NO: 6.

37. The self-replicating RNA molecule of claim 24, encoding a fusion protein comprising the truncated HBV core antigen operably linked to the HBV polymerase antigen.

38. The self-replicating RNA molecule of claim 37, wherein the fusion protein comprises the truncated HBV core antigen operably linked to the HBV polymerase antigen via a linker.

39. The self-replicating RNA molecule of claim 38, wherein the linker comprises the amino acid sequence of (AlaGly)n, and n is an integer of 2 to 5.

40. The self-replicating RNA molecule of claim 39, wherein the fusion protein comprises the amino acid sequence of SEQ ID NO: 16.

41. The self-replicating RNA molecule of claim 23, wherein the self-replicating RNA is an alphavirus-derived RNA replicon.

42. A composition comprising the self-replicating RNA of claim 23 and a pharmaceutically acceptable carrier.

43. The composition of claim 42, wherein the self-replicating RNA molecule is encapsulated in, bound to or adsorbed on a liposome, a lipoplex, a lipid nanoparticle, or combinations thereof.

44. A self-replicating RNA molecule comprising:
a) nonstructural genes nsP1, nsP2, nsP3 and nsP4;
b) a DLP motif;
c) a coding sequence for an autoprotease peptide operably linked downstream of the DLP motif;
d) a subgenomic promoter; and
e) a non-naturally occurring polynucleotide sequence encoding a Hepatitis B virus (HBV) polymerase antigen consisting of an amino acid sequence that is at least 98% identical to SEQ ID NO: 7, wherein the HBV polymerase antigen does not have reverse transcriptase activity and RNase H activity and is capable of inducing a T cell response against at least HBV genotypes B, C and D,
wherein the subgenomic promoter is operably linked to the non-naturally occurring polynucleotide sequence encoding the HBV polymerase antigen.

45. The self-replicating RNA molecule of claim 23, further comprising a non-naturally occurring polynucleotide sequence encoding a truncated HBV core antigen consisting of an amino acid sequence that is at least 95% identical to SEQ ID NO 2.

46. A method of treating a hepatitis B virus (HBV) infection or an HBV-induced disease in a subject in need thereof, comprising administering to the subject the self-replicating RNA molecule of claim 23.
